(19) United States
(12) Patent Application Publication
(10) Pub. No.: US 2024/0190950 A1
(43) Pub. Date: Jun. 13, 2024

(54) METHOD OF TREATING DISEASES USING GREMLIN1 ANTAGONISTS

(71) Applicants: SHANGHAI JIAO TONG UNIVERSITY, Shanghai (CN); SUZHOU TRANSCENTA THERAPEUTICS CO., LTD., Suzhou, Jiangsu (CN)

(72) Inventors: He ZHU, Shanghai (CN); Xueming QIAN, Suzhou, Jiangsu (CN); Weiqiang GAO, Shanghai (CN); Chaping CHENG, Shanghai (CN); Jinming WANG, Shanghai (CN); Di SUN, Suzhou, Jiangsu (CN)

(21) Appl. No.: 18/549,928

(22) PCT Filed: Mar. 11, 2022
(86) PCT No.: PCT/CN2022/080297

§ 371 Date: Sep. 11, 2023

(30) Foreign Application Priority Data
Mar. 11, 2021 (WO) PCT/CN2021/080142
Feb. 16, 2022 (WO) PCT/CN2022/076516

(51) Int. Cl. C07K 16/22; A61K 39/00; A61K 45/06; A61P 35/00

(52) U.S. Cl. CPC C07K 16/22; A61K 45/06; A61P 35/00; A61K 2039/505; C07K 2317/24; C07K 2317/31; C07K 2317/52; C07K 2317/565; C07K 2317/76

(57) ABSTRACT

The present disclosure provides herein treatment methods for GREM1-related disease or condition characterized in deficient in PTEN and/or p53. Also provided by the present disclosure are treatment methods for castration-resistant prostate cancers

### FIELD OF THE INVENTION

The present disclosure generally relates to a novel method of treating GREM1 related diseases using GREM1 antagonist.

### BACKGROUND

Gremlin1 is a highly conserved secreted protein in the DAN family of BMP antagonists. It was reported to bind to BMP-2, BMP-4 or BMP-7 to form heterodimers and prevent BMP ligands from interacting with the corresponding BMP receptors, then subsequently to inhibit the activation of BMP signaling. Gremlin1 is a pivotal protein during embryogenesis, and is closely related to tissue fibrotic lesions as well as glioma and colon cancer. However, our understanding of Gremlin1, as a secreted protein, is far from in-depth. Besides the BMP signaling pathway, whether Gremlin1 exerts its function through non-BMP mechanism has not been elucidated.

Therefore, there exists needs for exploration of novel medical uses of Gremlin1 targeting agents.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one aspect, the present disclosure provides a method of treating a GREM1-expressing disease or condition in a subject in need thereof, comprising administering to the subject a therapeutically effective amount of GREM1 antagonist, wherein the disease or condition is characterized in reduced or inhibited androgen receptor (AR) signaling.

In certain embodiments, the subject is receiving or has received an AR inhibitor. In certain embodiments, the disease or condition is resistant to an AR inhibitor.

In certain embodiments, the disease or condition is AR-associated cancer (such as prostate cancer, breast cancer, glioblastoma, melanoma, bladder cancer, renal cell carcinoma, pancreatic cancer, hepatocellular carcinoma, ovarian cancer, endometrial cancer, mantle cell lymphoma, or salivary gland cancer), or AR-associated non-cancer conditions (such as, hair loss, acne, hirsutism, ovarian cysts, polycystic ovary disease, precocious puberty, spinal and bulbar muscular atrophy, or age-related macular degeneration).

In one aspect, the present disclosure provides a method of treating a GREM1-expressing cancer in a subject in need thereof, comprising administering to the subject a therapeutically effective amount of GREM1 antagonist, wherein the cancer is characterized in reduced androgen receptor (AR) signaling.

In some embodiments, the cancer is an AR-expressing cancer or is an AR negative cancer.

In some embodiments, the cancer is prostate cancer, breast cancer, lung cancer, head and neck cancer, testis cancer, endometrial cancer, ovarian cancer, and skin cancer.

In some embodiments, the subject is receiving or has received an androgen deprivation therapy, or is resistant to an androgen deprivation therapy.

In some embodiments, the cancer is further determined to be deficient in PTEN and/or p53.

In some embodiments, the cancer is metastatic. In some embodiments, the cancer is metastatic prostate cancer.

In some embodiments, the cancer is lung metastasis of a cancer. In some embodiments, the cancer is lung metastasis of prostate cancer.

In some embodiments, the cancer is prostate cancer. In some embodiments, the prostate cancer is: a) negative in androgen receptor (AR) expression, b) negative in both androgen receptor (AR) expression and neuroendocrine (NE) differentiation; c) resistant to an androgen deprivation therapy, optionally castration-resistant, d) showing a level of Prostate Specific Antigen (PSA) lower than a reference level, or e) any combinations of a) to d).

In some embodiments, the cancer is characterized in GREM1 overexpression.

In one aspect, the present disclosure provides a method of increasing sensitivity of an AR-expressing cancer to an androgen deprivation therapy in a subject, comprising administering to the subject a therapeutically effective amount of GREM1 antagonist.

In one aspect, the present disclosure provides a method of treating a GREM1-related disease or condition characterized in deficiency in PTEN and/or p53 in a subject in need thereof, or inhibiting FGFR1 activation in a subject in need thereof, or inhibiting MAPK signaling in a subject in need thereof, comprising administering to the subject a therapeutically effective amount of GREM1 antagonist.

In some embodiments, the deficiency in PTEN and/or p53 is characterized in absence of functional PTEN and/or p53.

In some embodiments, the deficiency in PTEN and/or p53 is characterized in the presence of inactivating mutation in PTEN and/or p53.

In some embodiments, the deficiency in PTEN and/or p53 is characterized in absence of PTEN and/or p53 expression.

In some embodiments, the GREM1 related disease or condition is characterized in GREM1 expression or overexpression.

In some embodiments, the GREM1-related disease or condition is selected from the group consisting of cancer, fibrotic disease, angiogenesis, glaucoma or retinal disease, kidney disease, pulmonary arterial hypertension, and osteoarthritis (OA).

In some embodiments, the GREM1-related disease or condition is cancer.

In some embodiments, the cancer is prostate cancer, breast cancer, glioma, liposarcoma, hepatocellular carcinoma, lung cancer, cervical cancer, endometrial carcinoma, ulterine leiomyosarcoma, squamous cell carcinoma of the head and neck, thyroid cancer, liver cancer, pancreatic cancer, bladder cancer, colon cancer, esophageal cancer, bile duct cancer, osteosarcoma, glioblastoma, ovarian cancer, gastric cancer, triple negative breast cancer (TNBC), small cell lung cancer or melanoma.

In some embodiments, the cancer is prostate cancer.

In some embodiments, the prostate cancer is: a) negative in androgen receptor (AR) expression, b) negative in both androgen receptor (AR) expression and neuroendocrine (NE) differentiation; c) resistant to an androgen deprivation therapy, optionally castration-resistant, d) showing a level of Prostate Specific Antigen (PSA) lower than a reference level, or e) any combinations of a) to d).

In some embodiments, the cancer is breast cancer.

In some embodiments, the breast cancer is triple negative breast cancer.

In some embodiments, the fibrotic disease is lung fibrosis, skin fibrosis, diabetic nephropathy, or ischaemic renal injury.

In some embodiments, the GREM1 antagonist reduces GREM1 level or GREM1 activity.

In some embodiments, the GREM1 antagonist reduces the GREM1 activity selectively in cancer cell over in non-cancer cell.

In some embodiments, the GREM1 antagonist comprises an anti-GREM1 antibody or antigen-binding fragment thereof, an inhibitory GREM1 mimetic peptide, an inhibitory nucleic acid targeting GREM1 RNA or DNA, a polynucleotide encoding the inhibitory nucleic acid, a compound inhibiting interaction between gremlin and BMP, a compound inhibiting the GREM1 activity.

In some embodiments, the inhibitory nucleic acid targeting GREM1 RNA or DNA comprises a short hairpin RNA (shRNA), micro interfering RNA (miRNA), double strand RNA (dsRNA), small interfering RNA (siRNA), guide RNA, or antisense oligonucleotide.

In some embodiments, the GREM1 antagonist comprises a GREM1-FGFR1 axis inhibitor.

In some embodiments, the GREM1-FGFR1 axis inhibitor inhibits GREM1 dependent FGFR1 signaling.

In some embodiments, the GREM1-FGFR1 axis inhibitor blocks binding between GREM1 and FGFR1.

In some embodiments, the GREM1-FGFR1 axis inhibitor comprises an FGFR1-binding inhibitor.

In some embodiments, the FGFR1-binding inhibitor binds to extracellular domain 2 of FGFR1, and optionally binds to FGFR1 at an epitope comprising residue Glu 160, wherein residue number is according to SEQ ID NO: 75.

In some embodiments, the GREM1-FGFR1 axis inhibitor binds to hGREM1 at an epitope comprising residue Lys 123 and/or residue Lys 124, wherein residue number is according to SEQ ID NO: 69; or blocks FGFR1 binding to the residue Lys 123 and/or residue Lys 124 of hGREM1.

In some embodiments, the GREM1 antagonist or GREM1-FGFR1 axis inhibitor comprises an antibody against hGREM1 or an antigen-binding fragment thereof.

In some embodiments, the antibody comprises at least one of the following characteristics: a) capable of reducing hGREM1-mediated inhibition on BMP signaling selectively in a cancer cell over a non-cancer cell; b) exhibiting no more than 50% reduction of hGREM1-mediated inhibition on BMP signaling in a non-cancer cell; c) capable of binding to a chimeric hGREM1 comprising an amino acid sequence of SEQ ID NO: 68; d) capable of binding to hGREM1 but not specifically binding to mouse gremlin1; e) binding to hGREM1 at an epitope comprising residue Gln27 and/or residue Asn33, wherein residue number is according to SEQ ID NO: 69, or binds to a hGREM1 fragment comprising residue Gln27 and/or residue Asn33, optionally the hGREM1 fragment has a length of at least 3 (e.g. 4, 5, 6, 7, 8, 9, or 10) amino acid residues; f) capable of reducing hGREM1-mediated activation on MAPK signaling; and/or g) capable of binding to hGREM1 at a KD of no more than 1 nM as measured by Fortebio.

In some embodiments, the antibody comprises a linear epitope or a conformational epitope.

In some embodiments, the anti-GREM1 antibody or antigen-binding fragment thereof comprises a heavy chain variable (VH) region and/or a light chain variable (VL) region, wherein the heavy chain variable region comprises: a) a heavy chain complementarity determining region 1 (HCDR 1) comprises a sequence selected from the group consisting of SEQ ID NOs: 1, 11, 21 and 31, b) a HCDR2 comprises a sequence selected from the group consisting of SEQ ID NOs: 2, 12, 22 and 32, and c) a HCDR3 comprises a sequence selected from the group consisting of SEQ ID NOs: 3, 13, 23 and 33, and/or wherein the light chain variable region comprises: d) a light chain complementarity determining region 1 (LCDR1) comprises a sequence selected from the group consisting of SEQ ID NOs: 4, 14, 24 and 34, e) a LCDR2 comprises a sequence selected from the group consisting of SEQ ID NOs: 5, 15, 25 and 35, and f) a LCDR3 comprises a sequence selected from the group consisting of SEQ ID NOs: 6, 16, 26 and 36.

In some embodiments, the heavy chain variable region is selected from the group consisting of: a) a heavy chain variable region comprising a HCDR1 comprising the sequence of SEQ ID NO: 1, a HCDR2 comprising the sequence of SEQ ID NO: 2, and a HCDR3 comprising the sequence of SEQ ID NO: 3; b) a heavy chain variable region comprising a HCDR1 comprising the sequence of SEQ ID NO: 11, a HCDR2 comprising the sequence of SEQ ID NO: 12, and a HCDR3 comprising the sequence of SEQ ID NO: 13; c) a heavy chain variable region comprising a HCDR1 comprising the sequence of SEQ ID NO: 21, a HCDR2 comprising the sequence of SEQ ID NO: 22, and a HCDR3 comprising the sequence of SEQ ID NO: 23; and d) a heavy chain variable region comprising a HCDR1 comprising the sequence of SEQ ID NO: 31, a HCDR2 comprising the sequence of SEQ ID NO: 32, and a HCDR3 comprising the sequence of SEQ ID NO: 33.

In some embodiments, the light chain variable region is selected from the group consisting of: a) a light chain variable region comprising a LCDR1 comprising the sequence of SEQ ID NO: 4, a LCDR2 comprising the sequence of SEQ ID NO: 5, and a LCDR3 comprising the sequence of SEQ ID NO: 6; b) a light chain variable region comprising a LCDR1 comprising the sequence of SEQ ID NO: 14, a LCDR2 comprising the sequence of SEQ ID NO: 15, and a LCDR3 comprising the sequence of SEQ ID NO: 16; c) a light chain variable region comprising a LCDR1 comprising the sequence of SEQ ID NO: 24, a LCDR2 comprising the sequence of SEQ ID NO: 25, and a LCDR3 comprising the sequence of SEQ ID NO: 26; and d) a light chain variable region comprising a LCDR1 comprising the sequence of SEQ ID NO: 34, a LCDR2 comprising the sequence of SEQ ID NO: 35, and a LCDR3 comprising the sequence of SEQ ID NO: 36.

In some embodiments, a) the heavy chain variable region comprises a HCDR1 comprising the sequence of SEQ ID NO: 1, a HCDR2 comprising the sequence of SEQ ID NO: 2, and a HCDR3 comprising the sequence of SEQ ID NO: 3; and the light chain variable region comprises a LCDR1 comprising the sequence of SEQ ID NO: 4, a LCDR2 comprising the sequence of SEQ ID NO: 5, and a LCDR3 comprising the sequence of SEQ ID NO: 6; b) the heavy chain variable region comprises a HCDR1 comprising the sequence of SEQ ID NO: 11, a HCDR2 comprising the sequence of SEQ ID NO: 12, and a HCDR3 comprising the sequence of SEQ ID NO: 13; and the light chain variable region comprises a LCDR1 comprising the sequence of SEQ ID NO: 14, a LCDR2 comprising the sequence of SEQ ID NO: 15, and a LCDR3 comprising the sequence of SEQ ID NO: 16; c) the heavy chain variable region comprises a HCDR1 comprising the sequence of SEQ ID NO: 21, a HCDR2 comprising the sequence of SEQ ID NO: 22, and a HCDR3 comprising the sequence of SEQ ID NO: 23; and the light chain variable region comprises a LCDR1 comprising the sequence of SEQ ID NO: 24, a LCDR2 comprising the sequence of SEQ ID NO: 25, and a LCDR3 comprising the sequence of SEQ ID NO: 26; or d) the heavy chain variable region comprises a HCDR1 comprising the sequence of SEQ ID NO: 31, a HCDR2 comprising the sequence of SEQ ID NO: 32, and a HCDR3 comprising the sequence of SEQ ID NO: 33; and the light chain variable region comprises a LCDR1 comprising the sequence of SEQ ID NO: 34, a LCDR2 comprising the sequence of SEQ ID NO: 35, and a LCDR3 comprising the sequence of SEQ ID NO: 36.

In some embodiments, the heavy chain variable region comprises a sequence selected from the group consisting of SEQ ID NO: 7, SEQ ID NO: 17, SEQ ID NO: 27, SEQ ID NO: 37, SEQ ID NO: 41, SEQ ID NO: 43, SEQ ID NO: 45, SEQ ID NO: 51, SEQ ID NO: 53, SEQ ID NO: 55 and SEQ ID NO: 57, and a homologous sequence thereof having at least 80% sequence identity yet retaining specific binding specificity or affinity to gremlin.

In some embodiments, the light chain variable region comprises a sequence selected from the group consisting of SEQ ID NO: 8, SEQ ID NO: 18, SEQ ID NO: 28, SEQ ID NO: 38, SEQ ID NO: 47, SEQ ID NO: 49, SEQ ID NO: 59 and SEQ ID NO: 61, and a homologous sequence thereof having at least 80% sequence identity yet retaining specific binding specificity or affinity to gremlin.

In some embodiments, the anti-GREM1 antibody or antigen-binding fragment thereof comprising: a) a heavy chain variable region comprising the sequence of SEQ ID NO: 7 and a light chain variable region comprising the sequence of SEQ ID NO: 8; or b) a heavy chain variable region comprising a sequence of SEQ ID NO: 17 and a light chain variable region comprising a sequence of SEQ ID NO: 18; or c) a heavy chain variable region comprising a sequence of SEQ ID NO: 27 and a light chain variable region comprising a sequence of SEQ ID NO: 28; or d) a heavy chain variable region comprising a sequence of SEQ ID NO: 37 and a light chain variable region comprising a sequence of SEQ ID NO: 38; or e) a heavy chain variable region comprising a sequence selected from the group consisting of SEQ ID NO: 41, SEQ ID NO: 43 and SEQ ID NO: 45, and a light chain variable region comprising a sequence selected from the group consisting of SEQ ID NO: 47 and SEQ ID NO: 49; or f) a pair of heavy chain variable region and light chain variable region sequences selected from the group consisting of: SEQ ID NOs: 41/47, 41/49, 43/47, 43/49, 45/47, and 45/49; or g) a heavy chain variable region comprising a sequence selected from the group consisting of SEQ ID NO: 51, SEQ ID NO: 53, SEQ ID NO: 55 and SEQ ID NO: 57, and a light chain variable region comprising a sequence selected from the group consisting of SEQ ID NO: 59 and SEQ ID NO: 61; or h) a pair of heavy chain variable region and light chain variable region sequences selected from the group consisting of: SEQ ID NOs: 51/59, 51/61, 53/59, 53/61, 55/59, 55/61, 57/59, and 57/61.

In some embodiments, the anti-GREM1 antibody or antigen-binding fragment thereof further comprising one or more amino acid residue substitutions or modifications yet retains specific binding specificity or affinity to GREM1.

In some embodiments, at least one of the substitutions or modifications is in one or more of the CDR sequences, and/or in one or more of the non-CDR regions of the VH or VL sequences.

In some embodiments, the anti-GREM1 antibody or antigen-binding fragment thereof further comprising an immunoglobulin constant region, optionally a constant region of human Ig, or optionally a constant region of human IgG.

In some embodiments, the constant region comprises a constant region of human IgG1, IgG2, IgG3, or IgG4.

In some embodiments, the anti-GREM1 antibody or antigen-binding fragment thereof is humanized.

In some embodiments, the anti-GREM1 antibody or antigen-binding fragment thereof is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

In some embodiments, the anti-GREM1 antibody or antigen-binding fragment thereof is bispecific.

In one aspect, the present disclosure provides an antibody or antigen-binding fragment thereof, capable of specifically binding to a first and a second epitope of gremlin, or capable of specifically binding to both hGREM1 and a second antigen.

In one aspect, the present disclosure provides an antigen-binding fragment thereof, wherein the second antigen comprises an immune related target.

In one aspect, the present disclosure provides an antigen-binding fragment thereof, wherein the second antigen comprises PD-1, PD-L1, PD-L2, CTLA-4, TIM-3, LAG3, A2AR, CD160, 2B4, TGF B, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 or CD83.

In one aspect, the present disclosure provides an antigen-binding fragment thereof, wherein the second antigen comprises a tumor antigen.

In one aspect, the present disclosure provides an antigen-binding fragment thereof, wherein the tumor antigen comprises a tumor specific antigen or a tumor associated antigen.

In one aspect, the present disclosure provides an antigen-binding fragment thereof, wherein the tumor antigen comprises prostate specific antigen (PSA), CA-125, gangliosides G(D2), G(M2) and G(D3), CD20, CD52, CD33, Ep-CAM, CEA, bombesin-like peptides, HER2/neu, epidermal growth factor receptor (EGFR), erbB2, erbB3/HER3, erbB4, CD44v6, Ki-67, cancer-associated mucin, VEGF, VEGFRs (e.g., VEGFR-1, VEGFR-2, VEGFR-3), estrogen receptors, Lewis-Y antigen, TGFß1, IGF-1 receptor, EGFa, c-Kit receptor, transferrin receptor, Claudin 18.2, GPC-3, Nectin-4, ROR1, methothelin, PCMA, MAGE-1, MAGE-3, BAGE, GAGE-1, GAGE-2, pl5, BCR-ABL, E2APRL, H4-RET, IGH-IGK, MYL-RAR, IL-2R, CO17-1A, TROP2, or LIV-1.

In some embodiments, the anti-GREM1 antibody or antigen-binding fragment thereof is not cross-reactive to mouse GREM1.

In some embodiments, the anti-GREM1 antibody or antigen-binding fragment thereof is cross-reactive to mouse GREM1.

In some embodiments, the method further comprises administering a therapeutically effective amount of a second therapeutic agent.

In some embodiments, the second therapeutic agent comprises an anti-cancer therapy, optionally the anti-cancer therapy is selected from a chemotherapeutic agent, radiation therapy, an immunotherapy agent, anti-angiogenesis agent (e.g. antagonist of a VEGFR such as VEGFR-1, VEGFR-2, and VEGFR-3), a targeted therapy agent, a cellular therapy agent, a gene therapy agent, a hormonal therapy agent, cytokines, palliative care, surgery for the treatment of cancer (e.g., tumorectomy), one or more anti-emetics, treatments for complications arising from chemotherapy, or a diet supplement for cancer patients (e.g. indole-3-carbinol).

In some embodiments, the anti-cancer therapy comprises an anti-prostate cancer drug, optionally an androgen deprivation therapy.

In some embodiments, the anti-prostate cancer drug comprises an androgen axis inhibitor; an androgen synthesis inhibitor; a PARP inhibitor; or a combination thereof.

In some embodiments, the androgen axis inhibitor is selected from the group consisting of Luteinizing hormone-releasing hormone (LHRH) agonists, LHRH antagonists and androgen receptor antagonist.

In some embodiments, the androgen axis inhibitor is degarelix, bicalutamide, flutamide, nilutamide, apalutamide, darolutamide, enzalutamide, or abiraterone.

In some embodiments, the anti-prostate cancer drug is selected from the group consisting of Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Histrelin (Vantas), Jevtana (Cabazitaxel), Leuprolide Acetate, Lupron (Leuprolide Acetate), Lupron Depot (Leuprolide Acetate), Lynparza (Olaparib), Ketoconazole (Nizoral), Mitoxantrone Hydrochloride, Nilandron (Nilutamide), Nilutamide, Nubeqa (Darolutamide), Olaparib, Provenge (Sipuleucel-T), Radium 223 Dichloride, Relugolix (Orgovyx), Rubraca (Rucaparib Camsylate), Rucaparib Camsylate, Sipuleucel-T, Taxotere (Docetaxel), Triptorelin (Trelstar), Xofigo (Radium 223 Dichloride), Xtandi (Enzalutamide), Zoladex (Goserelin Acetate) and Zytiga (Abiraterone Acetate).

In one aspect, the present disclosure provides a method of determining likelihood of responsiveness to a GREM1 antagonist in a subject having or suspected of having cancer, comprising: (a) detecting androgen receptor (AR) expression or signaling in a biological sample from the subject, and (b) determining the likelihood of responsiveness based on the AR expression or signaling detected in step (a).

In some embodiments, the subject is determined to have likelihood of responsiveness to a GREM1 antagonist when the subject is detected to be absent in AR expression or signaling, or is detected to have reduced AR expression or signaling relative to a reference level.

In some embodiments, the method further comprises detecting GREM1 expression in a biological sample from the subject.

In some embodiments, the subject is determined to have likelihood of responsiveness to a GREM1 antagonist when the subject is detected to have GREM1 expression.

In one aspect, the present disclosure provides a method of detecting presence or amount of GREM1 in a sample determined to be absent in AR expression or determined to have reduced androgen receptor (AR) signaling, comprising contacting the sample with a detection reagent for detection of GREM1, and determining the presence or the amount of GREM1 in the sample.

In one aspect, the present disclosure provides a method of determining likelihood of responsiveness to a GREM1 antagonist in a subject having or suspected of having a disease or condition, comprising: (a) detecting deficiency of PTEN and/or p53 in a biological sample from the subject, and (b) determining the likelihood of responsiveness based on the deficiency of PTEN and/or p53 detected in step (a).

In some embodiments, the subject is determined to have likelihood of responsiveness to a GREM1 antagonist when the subject is detected to be deficient in PTEN and/or p53.

In some embodiments, the method further comprises detecting GREM1 expression in a biological sample from the subject.

In some embodiments, the subject is determined to have likelihood of responsiveness to a GREM1 antagonist when the subject is detected to have GREM1 expression.

In one aspect, the present disclosure provides a method of detecting presence or amount of GREM1 in a sample determined to be deficient in PTEN and/or p53, comprising contacting the sample with a detection reagent for detection of GREM1, and determining the presence or the amount of GREM1 in the sample.

In some embodiments, the sample is obtained from a subject having or suspected of having a GREM1 related disease or condition.

In some embodiments, the GREM1 related disease or condition is cancer, fibrotic disease, angiogenesis, glaucoma or retinal disease, kidney disease, pulmonary arterial hypertension, or osteoarthritis (OA).

In some embodiments, the cancer is prostate cancer, breast cancer, glioma, liposarcoma, hepatocellular carcinoma, lung cancer, cervical cancer, endometrial carcinoma, uterine leiomyosarcoma, squamous cell carcinoma of the head and neck, thyroid cancer, liver cancer, pancreatic cancer, bladder cancer, colon cancer, esophageal cancer, bile duct cancer, osteosarcoma, glioblastoma, ovarian cancer, gastric cancer, triple negative breast cancer (TNBC), small cell lung cancer or melanoma.

In some embodiments, the cancer is prostate cancer or breast cancer. wherein the prostate cancer is: a) resistant to an androgen deprivation therapy, optionally castration-resistant, and/or b) showing a level of Prostate Specific Antigen (PSA) lower than a reference level.

In some embodiments, the method further comprises administering a therapeutically effective amount of a GREM1 antagonist to the subject determined to have likelihood of responsiveness.

### BRIEF DESCRIPTION OF FIGURES

FIG. 1A shows immunohistochemical staining images of Gremlin1 in hormone sensitive PCas (HSPCs) (n=474) and castration-resistant prostate cancers (CRPCs) (n=60). Representative images are presented. Scale bars=100 μm.

FIG. 1B shows that GREM1 staining intensity is significantly higher in CRPCs than in HSPCs. Cytoplasm H score are analyzed with the Aperio ScanScope software.

FIG. 1C shows images of immunohistochemical staining of PSA and Gremlin1 in CRPCs. Scale bars=200 μm.

FIG. 1D shows that GREM1 mRNA transcription is downregulated by AR activation by R1881 (1 nM), and is significantly increased by AR inhibition by enzalutamide (10 μg/ml) in LNCaP cells.

FIG. 1E shows that Gremlin1 protein level is downregulated by AR activation by R1881 (1 nM), and is significantly increased by AR inhibition by enzalutamide (10 μg/ml) in LNCaP cells.

FIG. 1F shows that GREM1 promoter driven luciferase activity is downregulated by AR activation by R1881 (1 nM), and is significantly increased by AR inhibition by enzalutamide (10 μg/ml) in LNCaP cells. (Two-tailed Student's t test was used for the statistical analysis. *, P<0.05; **, P<0.01; ***, P<0.001. Data are presented as means±SEM.)

FIG. 1G shows chromatin immunoprecipitation (ChIP) assay results showing the enrichment levels of AR to the Gremlin1 promoter in LNCaP cells upon the treatment of R1881 or enzalutamide. Enz: enzalutamide. (Two-tailed Student's t test was used for the statistical analysis. *, P<0.05; **, P<0.01; ***, P<0.001. Data are presented as means±SEM.)

FIG. 1H shows a shorter overall survival in PCa patients with higher Gremlin1 expression (p<0.05).

FIG. 1I shows that GREM1 was most highly expressed in the prostate cancer tissue compared to other organs from Pbsn-Cre4; PTEN^{fl/fl}, Trp53^{fl/fl} mice (n=3).

FIG. 2A shows that compared to parental LNCaP cells, LNCaP castration resistant cells (LNCaP-R) display higher expression of Gremlin1.

FIG. 2B shows immunoblotting analysis of GREM1 expression in AR overexpressed LNCaP cells.

FIG. 2C shows q-PCR analysis of GREM1 expression in AR overexpressed LNCaP cells. (Two-tailed Student's t test was used for the statistical analysis. *, P<0.05; **, P<0.01; ***, P<0.001. Data are presented as means±SEM.)

FIG. 2D shows immunoblotting analysis of GREM1 expression in AR knockout LNCaP cells.

FIG. 2E shows q-PCR analysis of GREM1 expression in AR knockout LNCaP cells. (Two-tailed Student's t test was used for the statistical analysis. *, P<0.05; **, P<0.01; ***, P<0.001. Data are presented as means±SEM.)

FIG. 3A shows that immunoblotting confirms the efficiency of GREM1 knockdown or GREM1 overexpression in PC3 cells.

FIG. 3B shows that GREM1 knockdown leads to a suppression of sphere formation ability in PC3 cells, while GREM1 overexpression or addition of exogenous Gremlin1 protein display a promoting effect. Experiments were performed in triplicate.

FIG. 3C shows that GREM1 knockdown leads to a suppression of cell proliferation in PC3 cells, while GREM1 overexpression or addition of exogenous Gremlin1 protein display a promoting effect. Experiments were performed in triplicate.

FIG. 3D shows knockdown of GREM1 increases cell apoptosis in PC3 cells.

FIG. 3E shows that GREM1 knockdown represses PC3 xenograft growth in vivo.

FIG. 3F shows that GREM1 overexpression promotes PC3 xenograft forming incidence and tumor growth in vivo. Scale bars=1 cm.

FIG. 3G shows that the over-expression of Grem1 is verified by immunoblotting in Himyc mouse PCa derived organoid.

FIG. 3H shows that Gremlin1 promotes the organoid formation and androgen deprivation therapy (ADT) tolerance of the Himyc PCa organoids. (Two-tailed Student's t test and two-way ANOVA analysis were used for the statistical analysis. *, P<0.05; **, P<0.01; ***, P<0.001. Data are presented as means±SEM.)

FIG. 4A shows immunoblotting confirming the efficiency of GREM1 knockdown and overexpression in LNCaP cells.

FIG. 4B shows that GREM1 knockdown inhibits the sphere formation ability in LNCaP cells, while the overexpression of GREM1 or external addition of Gremlin1 protein exerts an opposite effect.

FIG. 4C shows that Gremlin1 promotes the growth of LNCaP PCa cells under androgen-deprivation therapy. ADT, androgen-deprivation therapy. Experiments were performed in triplicate.

FIG. 4D shows that knockdown of GREM1 increases cell apoptosis in LNCaP cells upon androgen-deprivation therapy. GREM1 expression and addition of exogenous Gremlin1 protein repress cell apoptosis in LNCaP cells treated with ADT. (Two-tailed Student's t test was used for the statistical analysis. *, P<0.05; **, P<0.01; ***, P<0.001. Data are presented as means±SEM.)

FIG. 5A shows immunoblotting confirming the efficiency of GREM1 overexpression in LAPC4 cells.

FIG. 5B shows that Gremlin1 enhances sphere forming of LAPC4.

FIG. 5C shows that Gremlin1 promotes the growth of LAPC4 cells under the ADT treatment.

FIG. 5D shows that GREM1 overexpression prevents cell death upon enzalutamide treatment characterized decreased Annexin V/PI staining. (Two-tailed Student's t test was used for the statistical analysis. *, P<0.05; **, P<0.01; ***, P<0.001. Data are presented as means±SEM.)

FIG. 6A shows gene set enrichment analysis of RNA-seq data demonstrates FGFR1 and MAPK signaling pathway are the most enriched signaling pathways in the GREM1 overexpressed LNCaP subline.

FIG. 6B shows gene set enrichment analysis of RNA-seq data demonstrates FGFR1 and MAPK signaling pathway are the most enriched signaling pathways in the GREM1 overexpressed LNCaP subline.

FIG. 6C shows that the FGFR/MEK/ERK signaling pathway is activated by Gremlin1 protein in PC3 cells and LNCaP-resistance cells in a dose dependent manner. FGF (20 ng/ml) is used as a positive control to stimulate FGFR.

FIG. 6D shows that activation of the MEK/ERK signaling pathway by Gremlin1 is independent on BMP4. PC3 cells and LNCaP-resistance cells were treated with Gremlin1 protein in the presence of BMP4 (20 ng/ml) or without BMP4.

FIG. 6E shows that Gremlin1 (100 ng/ml) treatment leads to a prolonged stimulation of the FGFR/MEK/ERK signaling activation than FGF (20 ng/ml) in PC3 cells.

FIG. 6F shows that Gremlin1 (100 ng/ml) treatment leads to a prolonged stimulation of the FGFR/MEK/ERK signaling activation than FGF (20 ng/ml) in LNCaP cells.

FIG. 6G shows that activation of FGFR/MEK/ERK signaling pathway is abrogated by CRISPR/Cas9 mediated FGFR1 knockout.

FIG. 6H shows that Gremlin1 activates the MEK/ERK signaling pathway through FGFR, wherein PC3 and LNCaP-resistance cells were treated with Gremlin1 (100 ng/ml), FGF1 (20 ng/ml), or a FGFR1/2/3 inhibitor BGJ398 (1 μM), as indicated.

FIG. 6I shows that the activation of MEK/ERK signaling pathway by Gremlin1 is independent on EGFR, wherein PC3 and LNCaP-resistance cells were treated with Gremlin1 (100 ng/ml), EGF (20 ng/ml), or an EGFR inhibitor Erlonitib (1 μM), as indicated.

FIG. 7A shows that compared to parental AR dependent LNCaP cells, LNCaP castration resistant cells (LNCaP R) show a strong activation of the FGFR1/MEK/ERK signaling pathway.

FIG. 7B shows that FGFR1/MEK/ERK signaling pathway is upregulated in the GREM1 overexpressed murine HiMyc PCa organoid compared to control organoids.

FIG. 8A shows that the promoting effects of Gremlin1 protein on LNCaP cells proliferation (n=6) are abrogated by FGFR1 or MEK inhibitors but are not affected by the addition of BMP4, wherein BGJ398 is an FGFR1 inhibitor and Trametinib is an MEK inhibitor.

FIG. 8B shows that the promoting effects of Gremlin1 protein on sphere forming (n=3) are abrogated by FGFR1 or MEK inhibitors but are not affected by the addition of BMP4, wherein BGJ398 is an FGFR1 inhibitor and Trametinib is an MEK inhibitor.

FIG. 8C shows immunoblotting results showing the efficiency of FGFR1 knockout in LNCaP cells.

FIG. 8D shows that FGFR1 knockout significantly attenuates the positive effects of Gremlin1 on LNCaP cells proliferation. (Two-tailed Student's t test was used for the statistical analysis. *, P<0.05; **, P<0.01; ***, P<0.001. Data are presented as means±SEM.).

FIG. 8E shows that FGFR1 knockout significantly attenuates the positive effects of Gremlin1 on sphere formation. (Two-tailed Student's t test was used for the statistical analysis. *, P<0.05; **, P<0.01; ***, P<0.001. Data are presented as means±SEM.).

FIG. 9A shows that FGFR1 binds to Gremlin1-biotin immobilized on Streptavidin sensor (ForBio) (Kd=1.06E-8M).

FIG. 9B shows that Predicted interaction of protein structures of Gremlin1 and FGFR1 extracellular domain imitated by Z-dock (http://zdock.umassmed.edu/). Protein structures are generated from PDB (http://www.rcsb.org/). Gremlin1:5AEJ; FGFR1:3ojv.

FIG. 9C shows that Gremlin1 co-immunoprecipitates with FGFR1 in 293T cells and LNCaP resistance cells transfected with flag-tagged Gremlin1 and HA-tagged FGFR1 expressing plasmids.

FIG. 9D shows that Endogenous Gremlin1 co-immunoprecipitates with FGFR1 in LNCaP-resistance cells.

FIG. 9E shows that Gremlin1 but not Gremlin 2 or other members of DAN protein family binds to FGFR1 as measured by Enzyme-linked immunosorbent assay (ELISA).

FIG. 9F shows that Interaction of purified Gremlin1 and soluble FGFR1 protein is demonstrated by pulldown experiments.

FIG. 9G shows that Soluble FGFR1 competitively inhibits the activation of FGFR1/MEK/ERK signaling by Gremlin1 in PC3.

FIG. 9H shows that BiFC assay shows colocalization between Gremlin1 and FGFR1 in LNCaP-resistance cells.

FIG. 9I shows immunofluorescent staining images of Gremlin1 and FGFR1 in LNCaP-R cells. The cells were treated with Gremlin1 (100 ng/ml) or PBS for 10 mins at 37° C.

FIG. 9J shows the diagram of truncated FGFR1.

FIG. 9K shows the Co-IP assay results between truncated FGFR1 and Gremlin1 (left panel) or FGF1 (right panel).

FIG. 9L shows the Gremlin1 mutagenesis strategies. Point mutations are bolded and underlined.

FIG. 9M shows the Gremlin1 K123A-K124A mutant disrupts the co-immunoprecipitation between Gremlin1 and FGFR1, wherein the numbering is relative to SEQ ID NO: 69.

FIG. 9N shows the schematic of FGFR1 mutations. Point mutations are bolded and underlined.

FIG. 9O shows that the co-immunoprecipitation of FGFR1 and Gremlin1 is impaired by the FGFR1 E160A mutation.

FIG. 9P shows schematics of FGFR1 mutations.

FIG. 9Q shows that FGFR1-C176G or FGFR1-R248Q mutation abolishes co-immunoprecipitation of FGF1 and FGFR1 (left panel), but do not influence the forming of protein complex between Gremlin1 and FGFR1 (right panel).

FIGS. 9R-9U show that the binding between Gremlin1 and FGFR1 is not affected by addition of FGF1, and vice versa, which are revealed by Fortebio (R), co-immunostaining (S) and pull-down (T, U) assays.

FIG. 9V shows docking module that highlights the key amino acid residues in the binding pocket between Gremlin1 and FGFR1.

FIG. 10A shows that binding specificity of anti-murine-Gremlin1 antibody to Gremlin1 is validated by the enzyme-linked immunosorbent assay. Ab is anti-murine-Gremlin1 in this figure.

FIG. 10B shows that Gremlin1 is highly expressed in the castrated Pbsn-Cre4; PTEN^{fl/fl}; Trp53^{fl/fl} murine PCa model. Representative images of Gremlin1 immunostaining are presented.

FIG. 10C shows that Anti-Gremlin1 antibody (10 μg/ml) exerts a significant inhibitory effect on PCa growth. Obvious suppression is found in gross tumor appearance.

FIG. 10D shows that Anti-Gremlin1 antibody (10 μg/ml) exerts a significant inhibitory effect on PCa growth. Obvious suppression is found in gross tumor weight.

FIG. 10E shows that Anti-Gremlin1 antibody (10 μg/ml) exerts a significant inhibitory effect on PCa growth. Obvious suppression is found in gross a significant reduction in PCNA positive cells.

FIG. 10F shows that Anti-Gremlin1 treatment markedly represses the development of invasive PCa in castrated Pbsn-Cre; PTEN^{fl/fl}; Trp53^{fl/fl} mice.

FIGS. 10G and 10H show that Gene set enrichment analysis indicates a significant suppression of the FGFR signaling pathway in the prostates of anti-Gremlin1 treatment group.

FIGS. 10I and 10J show that the immunostaining and immunoblot analysis show inhibitory effects of anti-Gremlin1 antibody on the FGFR1/MAPK signaling pathway in prostates of Phsn-Cre; PTEN^{fl/fl}; Trp53^{fl/fl} mice. (Two-tailed Student's t test was used for the statistical analysis. *, P<0.05; **, P<0.01; ***, P<0.001. Data are presented as means±SEM.).

FIG. 10K shows that the antibody against Gremlin1 (100 ng/ml) facilitates the inhibition of in vitro cell proliferation by enzalutamide (10 μg/ml) (n=3).

FIG. 10L shows that anti-Gremlin1 treatment suppresses the sphere formation ability of LNCaP-R cells (n=3).

FIG. 10M shows that the activation of FGFR1/MEK/ERK signaling pathway is suppressed by the Gremlin1 antibody in LNCaP-R cells.

FIG. 10N shows that annexin-V/DAPI staining demonstrates that anti-Gremlin1 antibody displays a synergistic effect with enzalutamide in inducing cell death. Ab: anti-human-Gremlin1. ADT: treated with enzalutamide at 10 μg/ml. (Two-tailed Student's t test was used for the statistical analysis. *, P<0.05; **, P<0.01; ***, P<0.001. Data are presented as means±SEM.)

FIG. 10O shows schematics illustrating the treatments in a Pbsn-Cre4; Ptenfl/fl; Trp53fl/fl GEMM. Mice which were castrated at 2 months received anti-Gremlin1 antibody (i.p., 10 mg/kg) or IgG, as indicated, three times a week for 2 months.

FIG. 11A shows that Gremlin was mainly expressed by the epithelial cells in castrated Pbsn-Cre; PTEN^{fl/fl}; Trp53^{fl/fl} PCa. ECAD. Ecadherin; VIM, Vimentin.

FIG. 11B shows that Gremlin1 antibody treatment does not induce major side effects when administered systemically to mice (10 mg/kg twice a week). No obvious alterations are detected in peripheral blood cell counts in mice received the antibody treatment. Ab: anti-mGREM1 antibody. (Two-tailed Student's t test was used for the statistical analysis. *, P<0.05; **, P<0.01; ***, P<0.001. Data are presented as means±SEM.)

FIG. 11C shows that Gremlin1 antibody treatment does not induce major side effects when administered systemically to mice (10 mg/kg twice a week). No obvious alterations are detected in major organs in mice received the antibody treatment. Ab: anti-mGREM1 antibody. (Two-tailed Student's t test was used for the statistical analysis. *, P<0.05; **, P<0.01; ***, P<0.001. Data are presented as means±SEM.)

FIG. 12A shows that binding specificity of the anti-human-Gremlin1 (14E3) to Gremlin1 is validated by the enzyme-linked immunosorbent assay. Ab is anti-human-Gremlin1 in this figure.

FIG. 12B shows that the antibody against human Gremlin1 (10 μg/ml) represses cell proliferation of PC3 cells.

FIG. 12C shows that anti-Gremlin1 antibody (10 μg/ml) exerts an inhibitory effect on sphere forming of PC3 cells.

FIG. 12D shows that Gremlin1 antibody neutralizes the activation of FGFR1/MEK/ERK signaling by Gremlin1 protein in PC3 cells in a dose-dependent manner.

FIGS. 12E and 12F show that anti-Gremlin1 treatment markedly impedes the in vivo growth of PC3 tumor xenografts in serial passage experiments. Antibody was given at indicated time points (see arrowhead) at 10 mg/kg via intra-peritoneal injection.

FIG. 13 shows treatment with 14E3 reduced tumor volume in PC3 CRPC model and increased percent survival.

FIG. 14A show that the antibody against Gremlin1 (100 ng/ml) facilitates the inhibition of in vitro cell proliferation by enzalutamide (1 ug/ml).

FIG. 14B shows that anti-Gremlin1 treatment suppresses the sphere formation ability of LNCaP cells.

FIG. 14C shows that the activation of FGFR1/MEK/ERK signaling pathway is suppressed by the Gremlin1 antibody in LNCaP cells

FIG. 14D shows that Annexin-V/DAPI staining demonstrates that anti-Gremlin1 antibody displays a synergistic effect with enzalutamide in inducing cell death. Ab: anti-human-Gremlin1 14E3. (Two-tailed Student's t test was used for the statistical analysis. *, P<0.05; **, P<0.01; ***, P<0.001. Data are presented as means±SEM.).

FIG. 15 shows 14E3 reduced the GREM1-mediated promotion on cancer cell migration.

FIG. 16A-16C show that 14E3 reduced the GREM1-mediated increase in the percentage of PSA-low population independent of the BMP-binding loop.

FIG. 17A shows that immunoblotting confirms the efficiency of BMPRII knockout in LNCaP cells.

FIGS. 17B and 17C show that BMPRII knockout showing no significant influence to the inhibitory effect of Gremlin1 antibody on LNCaP cell proliferation and sphere formation. Ab: anti-human-Gremlin1 14E3. (Two-tailed Student's t test was used for the statistical analysis. *, P<0.05; **, P<0.01; ***, P<0.001. Data are presented as means±SEM.)

FIG. 18A shows that immunofluorescent staining of AMCAR and DAPI indicates the tumor origin of patient derived organoids.

FIGS. 18B and 18C show that decreased organoid size and number suggests an inhibitory effect of the antibody against Gremlin1 14E3 on PDO forming and growth. (Two-tailed Student's t test was used for the statistical analysis. *, P<0.05; **, P<0.01; ***, P<0.001. Data are presented as means±SEM.).

FIG. 18D shows that detail information of patient samples used in this patient derived organoid experiment.

FIG. 19A shows heatmap images of tumors in each mouse model from the control group (mIgG2a) and the experiment group (14E3) respectively. Each of the control group and the experiment group has 16 mice.

FIG. 19B shows that the antibodies against Gremlin1 (e.g., 14E3) exerted no obvious influence on the body weight in the PCa metastasis mice model study.

FIG. 19C shows that the average radiance intensity was decreased with the Gremlin1 antibody treatment in the PCa metastasis mice model study.

FIG. 19D shows images of the lung tissue sections, where the arrows indicate metastases sites in the lung.

FIG. 19E shows the statistics of the number of micrometastases in lung in the PCa metastasis mice model study.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “inactivating mutation,” as used herein with respect to a biomarker provided herein such as AR, PTEN and/or p53, refers a mutation or a post-transcriptional modification that results in at least partial (or complete) loss of function or activity of the gene or of the gene product of biomarker (such as AR, PTEN and/or p53), or results in a non-functional gene or gene product. For example, the activity of the affected gene or gene product of the biomarker, would be significantly lower than wild-type counterpart or even be eliminated. An inactivating mutation can be a translocation, intragenic chromosome breaks, inversions, deletion (e.g., biallelic deletion, heterozygous or homozygous copy number loss), micro copy number alterations, insertion, substitution, aberrant splicing, or any combination thereof, which reduces the biological activity of the biomarker. In certain embodiments, insertion or deletion in a polynucleotide sequence may cause frame shift, which changes the reading frame of the codons and results in a completely different translated gene product from the original. This often generates truncated proteins that result in loss of function.

As used herein, the term “deletion” when used as a type of inactivating mutation of a biomarker, refers to a mutation in which one or more nucleobase pairs are lost or deleted from a polynucleotide sequence, or in which one or more amino acid residue are deleted from a polypeptide sequence. For example, it can refer to deletion, loss, or removal of an entire coding region or a portion thereof of the biomarker.

As used herein, a “substitution” is a mutation that exchanges one nucleobase for another in a polynucleotide sequence, or that substitutes one amino acid residue for another in a polypeptide sequence. Substitution in a polynucleotide sequence can: 1) change a codon to one that encodes a different amino acid residue, and therefore will cause change in amino acid sequence in the protein produced, or 2) change to a codon that encodes the same amino acid residue thereby causing no change in the protein produced; or 3) change an amino-acid-coding codon to a single “stop” codon and cause an incomplete protein (an incomplete protein is usually nonfunctional).

As used herein, an “insertion” is a mutation in which one or more extra nucleobase pairs are inserted into a place in a polynucleotide sequence, or in which one or more amino acid residue is inserted into a polypeptide sequence.

As used herein, a “translocation” refers to a type of chromosomal abnormality resulted from the exchange of genetic materials between two non-homologous chromosomes. A translocation may be either balanced or unbalanced; a balanced translocation results in no gain or loss of material, while an unbalanced translocation may result in trisomy or monosomy of a particular chromosome segment. Chromosomal translocations are typically seen in cases of leukemia, like, for instance, in acute myeloid leukemia.

The term “level” with respect to a biomarker such as AR, PTEN, and/or p53 refers to the amount or quantity of the biomarker of interest present in a sample. Such amount or quantity may be expressed in the absolute terms, i.e., the total quantity of the biomarker in the sample, or in the relative terms, i.e., the concentration or percentage of the biomarker in the sample. Level of a biomarker can be measured at DNA level (for example, as represented by the amount or quantity or copy number of the gene in a chromosomal region), at RNA level (for example as mRNA amount or quantity), or at protein level (for example as protein or protein complex amount or quantity).

As used herein, the term “reference level” with respect to a biomarker refers to a benchmark level which allows for comparison. A reference level may be chosen by the persons skilled in the art according to the desired purpose. Means for determining suitable reference levels are known to the persons skilled in the art, e. g. a reference level can be determined from experience, existing knowledge or data collected from clinical studies.

As used herein, the term “negative” with respect to a biomarker means that the biomarker is test negative or absent in a test sample. For example, the biomarker which is negative in a test sample may have a level comparable or undistinguishable from the negative control level in a sample lacking such a biomarker, or alternatively, may have a level below a threshold level that defines presence or a positive result.

As used herein, “likelihood” and “likely” with respect to response of a subject to a treatment is a measurement of how probable the therapeutic response is to occur in the subject. It may be used interchangeably with “probability”. Likelihood refers to a probability that is more than speculation, but less than certainty. Thus, a therapeutic response is likely if a reasonable person using common sense, training or experience concludes that, given the circumstances, a therapeutic response is probable.

The term “benefit from” or “responsive” as used in the context of therapy (e.g., treatment with a GREM1 antagonist) refers to beneficial or favorable response to the therapy, as opposed to unfavorable responses, i.e. adverse events.

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, and third constant region (C_{H1}, C_{H2}, C_{H3}, respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain). In certain embodiments, the antibody provided herein encompasses any antigen-binding fragments thereof.

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular parent antibody.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879 (1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, a “scFv dimer” is a bispecific diabody comprising V_{H}1-V_{L}2 (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} Or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or “dsFv-dsFv” comprises three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized gremlin antibody are substituted for sequence optimization. In certain embodiments, the variable region framework sequences of the humanized gremlin antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

The term “germline sequence” refers to the nucleic acid sequence encoding a variable region amino acid sequence or subsequence that shares the highest determined amino acid sequence identity with a reference variable region amino acid sequence or subsequence in comparison to all other known variable region amino acid sequences encoded by germline immunoglobulin variable region sequences. The germline sequence can also refer to the variable region amino acid sequence or subsequence with the highest amino acid sequence identity with a reference variable region amino acid sequence or subsequence in comparison to all other evaluated variable region amino acid sequences. The germline sequence can be framework regions only, complementarity determining regions only, framework and complementarity determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. Germline sequences can be determined, for example, through the publicly available international ImMunoGeneTics database (IMGT) and V-base.

“Anti-human gremlin1 antibody”, “anti-hGREM1 antibody” or “an antibody against human gremlin1” as used herein interchangeably and refers to an antibody that is capable of specific binding to human gremlin1 with a sufficient specificity and/or affinity, for example, to provide for therapeutic use.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. In certain embodiments, the antibodies or antigen-binding fragments provided herein specifically bind to human and/or non-human gremlin1 with a binding affinity (K_{D}) of ≤10⁶ M (e.g., ≤5×10⁻⁷ M, ≤2×10⁻⁷ M, ≤10⁻⁷ M, ≤5×10⁻⁸ M, ≤2×10⁻⁸ M, ≤108 M, ≤5×10⁻⁹ M, ≤4×10⁻⁹M, ≤3×10⁻⁹ M, ≤2×10⁻⁹ M, or ≤10⁻⁹ M. K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry method. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum correspondence. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm. In certain embodiments, the non-identical residue positions may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

As used herein, a “homologous sequence” refers to a polynucleotide sequence (or its complementary strand) or an amino acid sequence that has sequence identity of at least 80% (e.g. at least 85%, 88%, 90%, 91%, 92%, 93%, 94%, 95%, 96%, 97%, 98%, 99%) to another sequence when optionally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An isolated “nucleic acid” or “polynucleotide” are used interchangeably and refer to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “gremlin1” or “GREM1” refers to the variant 1 of gremlin, and encompasses gremlin1 in different species such as in human, mouse, monkey, and so on. GREM1 is evolutionarily conserved and the human gremlin1 gene (hGREM1) has been mapped to chromosome 15q13-q15 (Topol L Z et al., (1997) Mol. Cell Biol., 17: 4801-4810; Topol L Z et al., Cytogenet Cell Genet., 89: 79-84). The amino acid sequence of hGREM1 is accessibly by GenBank database under the accession number NP-037504 or Uniprot Database via the accession number 060565, and is provided herein as SEQ ID NO: 66. The term “human gremlin1” and the term “hGREM1” are used interchangeably in the present disclosure.

A “gremlin1-related” or “GREM1-related” disease or condition as used herein refers to any disease or condition caused by, exacerbated by, or otherwise linked to increased expression or activities of GREM1. In some embodiments, the GREM1 related condition is, for example, glaucoma, cancer, fibrotic disease, angiogenesis, retinal disease, kidney disease, pulmonary arterial hypertension, or osteoarthritis (OA).

“Cancer” as used herein refers to any medical condition characterized by malignant cell growth or neoplasm, abnormal proliferation, infiltration or metastasis, and can be benign or malignant, and includes both solid tumors and non-solid cancers (e.g. hematologic malignancies) such as leukemia. As used herein “solid tumor” refers to a solid mass of neoplastic and/or malignant cells.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “therapeutically effective amount” or “effective amount” means the amount of a pharmaceutical agent that that produces some desired local or systemic therapeutic effect at a reasonable benefit/risk ratio applicable to any treatment. When administered for preventing a disease, the amount is sufficient to avoid or delay onset of the disease. A therapeutically effective amount or an effective amount need not be curative or prevent a disease or condition from ever occurring. In certain embodiments, a therapeutically-effective amount of a pharmaceutical agent will depend on its therapeutic index, solubility, and the like.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

The present disclosure provides novel medical uses of gremlin 1 (GREM1) antagonists. The novel medical uses are, in part, based on the unexpected discovery that transcription of GREM1 is suppressed by androgen receptor (AR) and unleashed upon androgen deprivation therapy (ADT). The novel medical uses are, in part, based on the discovery that deficiency in PTEN and/or p53 promotes GREM1 expression. Furthermore, the present disclosure surprisingly discovered that GREM1 is significantly upregulated in advance prostate cancers including castration resistant prostate cancers (CRPCs), and positively correlates with development of castration resistance and poor overall survival. It has been shown by the inventors that GREM1 antagonists are useful in treating related conditions.

Methods of Treating GREM1-Expressing Conditions with Reduced Androgen Receptor Signaling

In one aspect, the present disclosure provides a method of treating a GREM1-expressing disease or condition in a subject in need thereof, comprising administering to the subject a therapeutically effective amount of GREM1 antagonist, wherein the disease or condition is characterized in reduced or inhibited androgen receptor (AR) signaling.

In certain embodiments, the subject is receiving or has received an AR inhibitor. In certain embodiments, the disease or condition is resistant to an AR inhibitor. AR inhibitor as used herein refers to a therapeutic agent useful in inhibiting AR activity, for example, those used in androgen deprivation therapy.

In certain embodiments, the disease or condition is AR-associated cancer (such as prostate cancer, breast cancer, glioblastoma, melanoma, bladder cancer, renal cell carcinoma, pancreatic cancer, hepatocellular carcinoma, ovarian cancer, endometrial cancer, mantle cell lymphoma, or salivary gland cancer), or AR-associated non-cancer conditions (such as, hair loss, acne, hirsutism, ovarian cysts, polycystic ovary disease, precocious puberty, spinal and bulbar muscular atrophy, or age-related macular degeneration).

In one aspect, the present disclosure provides methods of treating GREM1-expressing cancer in a subject in need thereof, comprising administering to the subject a therapeutically effective amount of GREM1 antagonist, wherein the cancer is characterized in reduced androgen receptor (AR) signaling.

### Reduced Androgen Receptor (AR) Signaling

Androgen receptor (AR) is a member of the steroid and nuclear receptor superfamily, and is mainly expressed in androgen target tissues, such as the prostate, skeletal muscle, liver, and central nervous system (CNS), with the highest expression level observed in the prostate, adrenal gland, and epididymis.

AR is a soluble protein that functions as an intracellular transcriptional factor. Upon binding and activation by androgens, AR mediates transcription of target genes that modulate growth and differentiation of prostate epithelial cells. AR signaling is crucial for the development and maintenance of male reproductive organs including the prostate gland.

As used herein, the term “reduced androgen receptor (AR) signaling” refers to AR signaling the level of which is substantially lower than the normal or baseline level of AR signaling, for example, a level of AR signaling in the healthy cell or tissue sample, or an average level of the AR signaling in the general cancer patient population or in a cancer patient population of a particular cancer of interest or in a patient population having AR dependent prostate cancers.

Cancer having reduced androgen receptor (AR) signaling can be an AR-expressing cancer, where the AR signaling is inhibited, for example, due to treatment (e.g. pharmacological treatment or surgical treatment), or due to reduced expression level of AR, or due to certain inactivating mutations in AR. Alternatively, the cancer having reduced AR signaling can be negative in AR expression, in particular for cancers that normally express AR (such as prostate cancer).

In some embodiment, the cancer is an AR-expressing cancer. Different types of cancers are known to express AR. Examples of AR-expressing cancer include without limitation, prostate cancer, breast cancer, lung cancer, head and neck cancer, testis cancer, endometrial cancer, ovarian cancer, and skin cancer. In certain embodiments, the AR-expressing cancer is prostate cancer or breast cancer.

In some embodiment, the subject is receiving or has received androgen deprivation therapy (ADT). The term “androgen deprivation therapy” or “ADT” as used herein refers to therapies that suppresses androgen, by reducing levels of androgen or by inhibiting biological functions of androgen such as by inhibiting AR signaling. The main androgens in the body are testosterone and dihydrotestosterone (DHT).

ADT can be achieved through surgical treatments (such as surgical castration) or drug treatments. Examples of ADT drugs include, without limitation, LHRH agonists (such as Leuprolide (Lupron, Eligard), Goserelin (Zoladex), Triptorelin (Trelstar), and Histrelin (Vantas)), LHRH antagonists (such as Degarelix (Firmagon), Relugolix (Orgovyx)), drugs that lower androgen levels from the adrenal glands (such as Abiraterone (Zytiga), Ketoconazole (Nizoral)), androgen receptor antagonists (such as Flutamide (Eulexin), Bicalutamide (Casodex), Nilutamide (Nilandron)), and other anti-androgens (such as Enzalutamide (Xtandi), apalutamide (Erleada) and darolutamide (Nubeqa)).

In some embodiment, the subject or the cancer is resistant to an ADT. By “resistant” it is meant that the disease has no or reduced responsiveness or sensitivity to an ADT. Reduced responsiveness can be indicated by, for example, requirement of an increased dose to achieve a given efficacy. In certain embodiments, the disease can be non-responsive to an ADT. For example, the cancer cells or tumor size increases despite of the treatment with the an ADT, or the disease showed regression back to its former state, for example, return of previous symptoms following partial recovery. The resistance to an ADT can be de novo or acquired.

In some embodiment, the subject or the cancer has reduced expression level of AR, or having one or more inactivating mutations in AR. Over 800 different AR mutations have been identified in patients with androgen insensitivity syndrome, and prostate cancer. In the AR gene, four different types of mutations have been detected to inactivate AR, including: a) single point mutations resulting in amino acid substitutions or premature stop codons; b) nucleotide insertions or deletions leading to a frame shift and premature rumination; c) complete or partial gene deletions; and d) intronic mutations causing alternative splicing (see, for details, K. Eisermann et al, Transl Androl, Urol. 2013 September; 2(3): 137-147).

In some embodiments, the cancer is negative in androgen receptor (AR) expression, i.e., AR-negative cancer. AR-negative cancer as used herein means a cancer originally having AR expression but becomes AR-negative. In certain embodiments, the AR-negative cancer is prostate cancer or breast cancer. Some prostate cancer cell lines are known to be AR-negative, such as PC3 cell line. An AR-negative cancer can be tested negative (or non-detectable) in AR expression or AR signaling, or can have a detected level of AR expression comparable to that of a known AR-negative prostate cancer cell.

In some embodiments, the prostate cancer or breast cancer is negative in both androgen receptor (AR) expression and neuroendocrine (NE) differentiation. NE differentiation in prostate cancer is a well-recognized phenotypic change by which prostate cancer cells transdifferentiate into NE-like cells. NE-like cells lack the expression of androgen receptor and prostate specific antigen, and are resistant to treatments. The NE differentiation can be assessed by measuring the protein level or mRNA level of NE markers chromogranin A (CgA), ratio of CgA/prostate specific antigen (PSA), and/or neuron specific enolsase (NSE). See, e.g., Hu et al., Front Oncol. 2015; 5:90; Berruti et al., Endocr Relat Cancer (2005) 12(1):109-17.10.1677/erc.1.00876; Khan et al., J Pak Med Assoc (2011) 61(1):108-11; Taplin et al., Urology (2005) 66(2):386-91.10.1016/j.urology; Sarkar et al., Cancer Biomark (2010) 8(2):81-7.10.3233/CBM-2011-0198; Burgio et al., Endocr Relat Cancer (2014) 21(3):487-93.10.1530/ERC-14-0071; Conteduca et al., Prostate (2014) 74(16):1691-6.10.1002/pros.22890; Berruti et al., Cancer (2000) 88(11):2590-7.10.1002/1097-0142(20000601)88:11; Sasaki et al., Eur Urol (2005) 48(2):224-9.10.1016/j.eururo.2005.03.017, disclosure of which are hereby incorporated by reference in their entirety.

In some embodiments, the prostate cancer is further characterized in having a level of Prostate Specific Antigen (PSA) lower than a reference level.

PSA is a classic downstream target of AR. Normally, very little PSA is secreted in the blood. Increases in glandular size and tissue damage caused by benign prostatic hypertrophy, prostatitis, or prostate cancer may increase circulating PSA levels. Prostate cancer cells at advanced stages that are poorly differentiated or undifferentiated produce less PSA and are accompanied with a low level of PSA (for example, less than 4 ng/ml). It is also believed that prostate cancer cells having low level of PSA or negative for PSA could be resistant to anti-androgens, chemotherapeutic drugs, pro-oxidants, or radiation, and may be castration-resistant (Skvortsov S. et al, STEM CELLS, Vol. 36, Issue 10, 1457-1474).

The reference level of PSA can be a threshold level of PSA normally found in a PSA positive prostate cancer. The reference level of PSA can also be an average level of the PSA in a general prostate cancer patient population or in a patient population having prostate cancers before progressing into advanced stages. Certain reference levels of PSA in blood can be, for example, about 2 ng/ml, about 4 ng/ml, about 6 ng/ml, about 8 ng/ml or about 10 ng/ml as measured using immunodetectable assays, e.g., the Hybritech (San Diego, Calif), Tosoh (Foster City, Calif), Bayer Centaur PSA Assay kit (Tarrytown, NY), or Abbott assays (Chicago, Ill). See, e.g., Dan et al., Cancer, Volume 109, Issue 2, 2007. https://doi.org/10.1002/cncr.22372; and Oesterling et al., J Urol. 1995; 154:1090-1095, disclosure of which are hereby incorporated by reference in their entirety.

In certain embodiments, the prostate cancer is negative for PSA. For example, the prostate cancer does not express PSA, or is tested to be negative in a test for PSA.

In some embodiment, the prostate cancer is castration-resistant. Castration-resistant prostate cancer (CRPC) is an advanced prostate cancer that is capable to grow despite of low levels of circulating androgens. CRPC may present as either a continuous rise in PSA levels, the progression of pre-existing disease, and/or the appearance of new metastasis, despite a serum testosterone value below 50 ng/dL after ADT (Toshiyuki Kamoto et al., Nihon Rinsho. 2014 December; 72(12):2103-7; Fred Saad et al., Can Urol Assoc J. 2010 December; 4(6): 380-384).

Some CRPC can remain dependent on AR signaling despite depletion or reduction of androgens. For example, CRPC can be developed via amplifying AR expression, mutating the AR gene and/or genes encoding coactivators/corepressors, activating androgen-independent AR pathways, and/or producing alternative androgen, so as to remain the dependency on AR pathway for disease progression (Thenappan et al., Transl Androl Urol. 2015 June; 4(3): 365-380.) Some CRPC can bypass the requirement for AR signaling.

In some embodiments, the prostate cancer is: a) negative in androgen receptor (AR) expression, b) negative in both androgen receptor (AR) expression and neuroendocrine (NE) differentiation; c) resistant to an androgen deprivation therapy, optionally castration-resistant, d) showing a level of Prostate Specific Antigen (PSA) lower than a reference level, or e) any combinations of a) to d).

In some embodiments, the cancer is further determined to be deficient in PTEN and/or p53.

In certain embodiments, the cancer is metastatic. A metastatic cancer can spread or has spread from its site of origin to another part of the body. A metastatic tumor is the same type of cancer as the primary tumor. A metastatic cancer may spread to areas near the primary site, or to distant parts of the body.

### GREM1 Overexpression

Without wishing to be bound by any theory, it is believed that AR signaling is negatively correlated with GREM1 expression, and reduced AR signaling is believed to result in increased expression of GREM1.

In some embodiment, the cancer having reduced AR signaling is further characterized in GREM1 expression or overexpression. The GREM1 expression or overexpression can be in a disease cell or in a disease microenvironment.

The term “overexpression” with respect to GREM1 as used herein refers to an increased expression level relative to a reference level. The reference level can be the level of GREM1 expression found in normal cells of the same tissue type, optionally normalized to expression level of another gene (e.g. a house keeping gene). Alternatively, the reference level can be the level of GREM1 expression found in healthy subjects. The expression level which can be determined based on nucleic acid level or protein level. In some embodiments, the GREM1-expressing cancer has a GREM1 expression level at least 10% higher (e.g. at least 15%, 20%, 30%, 35%, 40%, 50% or 1-fold, 2-fold, 3-fold or even higher) than a reference level.

### Methods of Increasing Sensitivity of an AR-Expressing Cancer to an Androgen Deprivation Therapy

In another aspect, the present disclosure further provides methods of increasing sensitivity of an AR-expressing cancer to an androgen deprivation therapy (ADT) in a subject, comprising administering to the subject a therapeutically effective amount of GREM1 antagonist.

Without wishing to be bound by any theory, it is believed that AR signaling reduction by ADT could lead to GREM1 expression or increased expression, and use of a GREM1 antagonist can further improve the sensitivity of AR-expressing cancer to an ADT.

The term “sensitivity” with regard to cancer refers to the ability of cancer to respond to a treatment (e.g., treatment with a GREM1 antagonist). Sensitivity of cancer can be measured in terms of, e.g., inhibition of cancer cell proliferation or promotion of cancer cell death. Increased sensitivity can be determined based on increased efficacy under the same dose, or reduction in dose for a similar efficacy.

In some embodiments, the methods comprises administering to the subject the GREM1 antagonist in combination with the ADT.

Methods of Treating GREM1-Related Disease or Condition Characterized in Deficient in PTEN and/or p53

In various embodiments, the present disclosure provides methods of treating a GREM1-related disease or condition characterized in deficiency of PTEN and/or p53 in a subject.

PTEN and/or p53 Deficiency

PTEN and p53 contribute to the regulation of self-renewal and differentiation in prostate progenitors and presumptive tumor initiating cells for prostate cancer. The term PTEN and/or p53 provided herein are intended to encompass different forms including mRNA, protein and also DNA (e.g. genomic DNA). Therefore, the level and/or activity and/or mutation status of PTEN and/or p53 can be measured with RNA (e.g. mRNA), protein or DNA (e.g. genomic DNA).

The term “TP53” and “p53” are used interchangeably herein. TP53 is a transcription factor capable of regulating a number of genes that regulate e.g. cell cycle and apoptosis. Alternative names for p53 include, e.g., antigen NY-CO-13, phosphorprotein p53, tumor suppressor p53 and cellular tumor antigen p53. p53 as used herein can indicate the TP protein as well as the polynucleotide (e.g. DNA or RNA) encoding the TP53 protein, including all isoforms and variants. In certain embodiments, the gene of p53 is available in GenBank database under the NCBI Reference Sequence of NG_017013.2, and exemplary sequence of human p53 protein is available in UniProtKB database under the accession number of P04637 (P53-HUMAN). In certain embodiments, the protein of p53 comprises an amino acid sequence of SEQ ID NO: 73.

The term “PTEN”, “Pten” and “PTEN tyrosine phosphatase” are used interchangeably herein. PTEN, also known as phosphatase and tensin homolog deleted on chromosome ten, is a tumor suppressor that acts as a dual-specificity protein phosphatase that antagonizes the PI3K signaling pathway through its lipid phosphatase activity and negatively regulates the MAPK pathway through its protein phosphatase activity (Pezzolesi et al., Hum. Molec. Genet. 16: 1058-1071, 2007.). PTEN as used herein can refer to the PTEN protein as well as the DNA (e.g. the coding gene sequence) or the RNA encoding for the PTEN, including all isoforms and variants. Exemplary sequence of human PTEN is available in UniProtKB database under the accession number of P60484 (PTEN_HUMAN), with three isoforms: isoform 1 (P60484-1), isoform alpha (P60484-2) and isoform 3 (P60484-3). Exemplary sequence of gene of PTEN is available in GenBank database under the NCBI Reference Sequence of NC_000010.11. In certain embodiments, the protein of PTEN comprises an amino acid sequence of SEQ ID NO: 74.

As used herein, “deficiency” or “deficient” refers to insufficiency in activity or level, and can include, for example, being less than normal activity or level, or being absent or null in activity or level. For example, deficiency in activity or level of PTEN and/or p53 can result in PTEN and/or p53 having no or less than normal function, or an absence of or reduced expression level of PTEN and/or p53 in a biological sample.

In certain embodiments, the deficiency in PTEN and/or p53 is characterized in absence of functional PTEN and/or p53.

In certain embodiments, the deficiency in activity or level of PTEN and/or p53 can be indicated by the presence of the inactivating mutation in PTEN and/or p53.

It is to be understood that the present disclosure is not limited to any specific PTEN or p53 mutations. Any inactivating mutations in PTEN or p53 can be useful in the present disclosure.

In certain embodiments, the deficiency in activity or level of PTEN and/or p53 can be indicated by the expression level or copy number of PTEN and/or p53 in the biological sample. Accordingly, to determine if there is deficiency in activity or level of PTEN and/or p53 in the biological sample, the methods provided herein can comprise the step of determining if expression level or copy number of PTEN and/or p53 is reduced in the biological sample relative to a reference level.

Mutation status or expression level of PTEN and/or p53 at DNA or RNA level can be measured by any methods known in the art, for example, without limitation, an amplification assay, a hybridization assay, or a sequencing assay. Mutation status or expression level of PTEN and/or p53 at protein level can be measured by any methods known in the art, for example, without limitation, immunoassays.

In certain embodiments, the deficiency in PTEN and/or p53 is characterized in absence of PTEN and/or p53 expression.

In certain embodiments, the deficiency in activity or level of PTEN and/or p53 can be indicated by epigenetic silencing, transcriptional repression, or microRNA (miRNA) regulation of PTEN and/or p53.

Without wishing to be bound by any theory, it is believed that deficiency in p53/PTEN, for example, by inactivating mutation, results in increased expression of GREM1. In some embodiment, the GREM1 related disease or condition characterized in deficiency in PTEN and/or p53 is further characterized in GREM1 expression or overexpression. GREM1 expression can be determined using methods provided above.

In certain embodiments, the subject is human. In certain embodiments, the subject is identified as having a GREM1 expression or overexpression, optionally in a biological sample obtained from the subject.

### GREM1-Related Disease or Condition

In some embodiment, the GREM1-related disease or condition is selected from the group consisting of cancer, fibrotic disease, angiogenesis, glaucoma or retinal disease, kidney disease, pulmonary arterial hypertension, and osteoarthritis (OA).

In some embodiment, the GREM1-related disease or condition is cancer. In certain embodiments, the cancer is metastatic cancer. In certain embodiments, the cancer is prostate cancer, breast cancer, glioma, liposarcoma, hepatocellular carcinoma, lung cancer, cervical cancer, endometrial carcinoma, ulterine leiomyosarcoma, squamous cell carcinoma of the head and neck, thyroid cancer, liver cancer, pancreatic cancer, bladder cancer, colon cancer, esophageal cancer, bile duct cancer, osteosarcoma, glioblastoma, ovarian cancer, gastric cancer, triple negative breast cancer (TNBC), small cell lung cancer or melanoma.

In some embodiment, the cancer is prostate cancer.

In some embodiment, the prostate cancer is: a) negative in androgen receptor (AR) expression, b) negative in both androgen receptor (AR) expression and neuroendocrine (NE) differentiation; c) resistant to an androgen deprivation therapy, optionally castration-resistant, d) showing a level of Prostate Specific Antigen (PSA) lower than a reference level, or e) any combinations of a) to d).

In some embodiment, the cancer is breast cancer. The breast cancer can be triple negative breast cancer.

In some embodiment, the fibrotic disease is lung fibrosis, skin fibrosis, Diabetic nephropathy, or ischaemic renal injury.

The method comprising administering to the subject a therapeutically effective amount of GREM1 antagonist. A GREM1-related disease or condition can be a disease or condition that would benefit from modulation of GREM1 activity (e.g. reduction in GREM1 activity). In some embodiment, the GREM1 related disease or condition is characterized in GREM1 expression or overexpression.

In some embodiments, the GREM1-related disease or condition characterized in deficiency in PTEN and/or p53 can be selected from the group consisting of cancer, fibrotic disease, angiogenesis, glaucoma or retinal disease, kidney disease, pulmonary arterial hypertension, and osteoarthritis (OA). Increased levels of GREM1 have been associated with many of these diseases and conditions, such as scleroderma, diabetic nephropathy, glioma, head and neck cancer, prostate cancer and colorectal cancer.

i. Cancer

In some embodiments, the GREM1-related disease or condition characterized in deficiency in PTEN and/or p53 is cancer, in particular, GREM1-expressing cancer.

The treatment methods provided herein are based on the surprising finding of a significant upregulation of GREM1 in cancer cells deficient in PTEN and/or p53 that was unknown before.

In certain embodiments, the cancer is selected from solid tumors or hematological tumors. In certain embodiments, the solid tumor is adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt's lymphoma, cervical cancer, colon cancer, emphysema, endometrial cancer, esophageal cancer, Ewing's sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, or vaginal cancer.

In certain embodiments, the hematological tumor is leukemia (such as Acute lymphocytic leukemia (ALL), Acute myeloid leukemia (AML), Chronic lymphocytic leukemia (CLL), Chronic myeloid leukemia (CML)), lymphoma (such as Hodgkin's lymphoma, or Non-Hodgkin's lymphoma (e.g. Waldenstrom macroglobulinemia (WM))), or myeloma (such as multiple myeloma (MM)). In certain embodiments, the cancer is multiple myeloma (MM). GREM1 is found to be abundantly secreted by a subset of bone marrow (BM) mesenchymal stromal cells, and is considered to play a critical role in MM disease development. Analysis of human and mouse BM stromal samples by quantitative PCR showed that GREM1/Grem1 expression was significantly higher in the MM tumor-bearing cohorts compared to healthy control. Anti-GREM1 antibodies have been shown to decrease MM tumor burden in mice (K. Clark et al., Cancers 2020, 12, 2149).

In certain embodiments, the cancer is prostate cancer, gastric-esophageal cancer, lung cancer (e.g., non-small cell lung cancer), liver cancer, pancreatic cancer, breast cancer, bronchial cancer, bone cancer, liver and bile duct cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, gastrointestinal cancer, skin cancer, pituitary cancer, stomach cancer, vagina cancer, thyroid cancer, glioblastoma, astrocytoma, melanoma, myelodysplastic syndrome, sarcoma, teratoma, glioma, adenocarcinoma, leukemia (such as Acute lymphocytic leukemia (ALL), Acute myeloid leukemia (AML), Chronic lymphocytic leukemia (CLL), Chronic myeloid leukemia (CML)), lymphoma (such as Hodgkin's lymphoma, or Non-Hodgkin's lymphoma (e.g. Waldenstrom macroglobulinemia (WM))), or myeloma (such as multiple myeloma (MM)), triple negative breast cancer (TNBC), small cell lung cancer, esophageal cancer, osteosarcoma, and gastric cancer.

In certain embodiments, the cancer is selected from the group consisting of prostate cancer, gastric-esophageal cancer, lung cancer (e.g., non-small cell lung cancer), liver cancer, colon cancer, colorectal cancer, glioma, pancreatic cancer, bladder cancer and breast cancer. In certain embodiments, the cancer is triple negative breast cancer. In certain embodiments, the cancer is multiple myeloma.

In certain embodiments, the cancer is metastatic. In certain embodiments, the present disclosure further provides methods of treating or preventing cancer metastasis using the antibodies provided herein. Cancer metastasis is the process during which cancer cells spread from its original site to another site within the body.

In certain embodiments, the cancer is prostate cancer, breast cancer or liver cancer. The tumor suppressors Pten and p53 are frequently lost in prostate cancer or breast cancer.

In certain embodiments, the breast cancer is triple negative breast cancer. The term “triple-negative breast cancer” or “TNBC” refers to a breast cancer that is tested negative for estrogen receptors, progesterone receptors, and excess HER2 protein. TNBC can be non-responsive to hormone therapies or drugs targeting HER2. The expression in a sample can be detected as mentioned above under the section Methods of Treating GREM1-related Prostate Cancer with Reduced Androgen Receptor Signaling.

TNBCs deficient in PTEN and/or p53 have worse prognosis compared to other TNBCs with normal level of these tumor suppressors (Jeff C. L., et al., EMBO Mol Med (2014)6:1542-1560). Combined Pten-p53 mutations are found to accelerate formation of claudin-low, triple-negative-like breast cancer (TNBC) that exhibited hyper-activated AKT signaling and more mesenchymal features relative to Pten or p53 single-mutant tumors.

In some embodiments, GREM1-related disease or condition characterized in deficiency of PTEN and/or p53 is liver cancer, e.g., hepatocellular carcinoma (HCC). In some embodiments, the liver cancer is Hepatitis B virus (HBV) infection related HCC. HCC is the second leading cause of cancer-related deaths in the world. Persistent HBV infection is one of the major risk factors for HCC development, which accounts for more than 50% of HCC worldwide. HBV infection related HCC can be developed via CRISPR/Cas9 mediated mutations of p53 and PTEN loci that leads to deficiency in PTEN and/or p53 (Yongzhen L., et al., Scientific Reports (2017) 7: 2796). The origin of HCCs has been considered as enhanced proliferation and maturation arrest of hepatic progenitor/stem cells, which was shown to be promoted by fibrosis via fibroblast-secreted GREM1 that blocks BMP function (Guimei M et al., BMC Res Notes 2012; 5:390.).

ii. Fibrotic Diseases

The PTEN and/or p53-deficient disease or condition may also be a non-cancer disease, as long as the disease is characterized in PTEN and/or p53 deficiency which is further associated with GREM1 upregulation. For example, non-cancer diseases such as lung and skin fibrosis and diabetic and ischaemic renal injury have been reported to involve dysregulation of p53 or PTEN, and these disease are also known to be associated with GREM1 expression (see, for details, Rohan Samarakoon et al., Loss of Tumour Suppressor PTEN Expression in Renal Injury Initiates SMAD3 and p53 Dependent Fibrotic Responses, J Pathol. 2015 August; 236(4): 421-432; Nagaraja M. R. et al, “p53 Expression in Lung Fibroblasts Is Linked to Mitigation of Fibrotic Lung Remodeling”, Am J Pathol. 2018 October; 188(10): 2207-2222.). The present inventors unexpectedly discovered the correlation between GREM1 and PTEN/p53, it is therefore expected that non-cancer disease or conditions characterized in deficiency in PTEN and/or p53 may also be treated by administering a GREM1-modulating agent, including a GREM1 antagonist.

In some embodiment, the GREM1-related disease or condition characterized in deficiency in PTEN and/or p53 is a fibrotic disease. Fibrotic disease is a disease or condition that involves fibrosis. Fibrosis is a scarring process that is a common feature of chronic organ injury, for example in lungs, liver, kidney, skin, heart, gut or muscle. Fibrosis is characterized by elevated activity of transforming growth factor-beta (TGF-β) resulting in increased and altered deposition of extracellular matrix and other fibrosis-associated proteins. Elevated GREM1 expression has been found in many fibrotic diseases, suggesting that GREM1 may be an important marker of fibrosis (Costello, et al., 2010, Am. J. Respir. Cell. Mol. Biol. 42: 517-523; Lappin, et al., 2002, Nephrol. Dial. Transplant. 17: 65-67; Boers et al., 2006, J. Biol. Chem. 281: 16289-16295).

Fibrotic disease can include fibrotic disease in lungs, liver, kidney, eyes, skin, heart, gut or muscle. Examples of fibrotic disease in lungs include pulmonary fibrosis, cystic fibrosis, pulmonary hypertension, progressive massive fibrosis, bronchiolitis obliterans, airway remodeling associated with chronic asthma or idiopathic pulmonary. Examples of fibrotic disease in liver include cirrhosis or non-alcoholic steatohepatitis. Examples of fibrotic disease in kidney include such as renal fibrosis, ischemic renal injury, tubulointerstitial fibrosis, diabetic nephropathy, nephrosclerosis, or nephrotoxicity. Examples of fibrotic disease in eyes include such as corneal fibrosis, subretinal fibrosis. Examples of fibrotic disease in skin include such as nephrogenic systemic fibrosis, keloid or scleroderma. Examples of fibrotic disease in heart include endomyocardial fibrosis or old myocardial infarction.

iii. Other Diseases

In some embodiment, the GREM1-related disease or condition is pulmonary artery hypertension (PAH). The term “pulmonary arterial hypertension” (“PAH”) refers to a progressive lung disorder which is characterized by sustained elevation of pulmonary artery pressure. GREM1 has been found to be elevated in the wall of small intrapulmonary vessels of mice during hypoxia. Anti-GREM1 antibodies have been found to alleviate or ameliorate one or more symptoms associated with PAH, for example, inhibits thickening of the pulmonary artery, increases stroke volume and/or stroke volume to end systolic volume ratio (“SV/ESV”), increases right ventricle cardiac output and/or cardiac index (CI), improve other hemodynamic measurements in a subject having PAH, such as, for example, right atrium pressure, pulmonary artery pressure, pulmonary capillary wedge pressure in the presence of end expiratory pressure, systemic artery pressure, heart beat, pulmonary vascular resistance, and/or systemic vascular resistance (see, for details, U.S. patent application US20180057580A1).

In some embodiment, the GREM1-related disease or condition is osteoarthritis (OA). GREM1 is reported as a mechanical loading-inducible factor in chondrocytes, and is detected at high levels in middle and deep layers of cartilage after cyclic strain or hydrostatic pressure loading. GREM1 is reported to be up-regulated in osteoarthritis, and GREM1 concentrations in serum and in synovial fluid are correlated with the onset and severity of knee OA (J. Yi, et al., Med Sci Monit, 2016; 22: 4062-4065). GREM1 activates nuclear factor-KB signaling, leading to subsequent induction of catabolic enzymes. Intra-articular administration of GREM1 antibody or chondrocyte-specific deletion of GREM1 in mice was reported to decelerate osteoarthritis development (see, S. H. Chang et al., Nature Communications, (2019) 10: 1442).

In some embodiment, the GREM1-related disease or condition is angiogenesis. GREM1 is an agonist of the major proangiogenic receptor vascular endothelial growth factor receptor-2 (VEGFR-2). Heparan sulfate (HS) and heparin, glycosaminoglycans (GAGs) known for their anticoagulant effects, have been shown to bind to GREM1. GREM1 binds to heparin and activates VEGFR-2 in a BMP-independent manner (Chiodelli et al 2011; Arterioscler. Thromb. Vasc. Biol. 31: e116-e127). Anti-GREM1 antibodies have been found to alleviate or ameliorate one or more symptoms associated with angiogenesis or heparin-mediated angiogenesis (see, for details, U.S. patent application US20200157194).

In some embodiment, the GREM1-related disease or condition is glaucoma. Glaucoma may be caused by altered expression of one or more BMP family genes in the eye, which leads to elevated increased intraocular pressure and/or glaucomatous optic neuropathy. GREM1 has been found to have an increased expression in glaucomatous trabecular meshwork cells. GREM1 antagonists have been found to alleviate or ameliorate one or more symptoms associated with angiogenesis or glaucoma (see, for details, U.S. patent U.S. Pat. No. 7,744,873).

In some embodiment, the GREM1-related disease or condition is retinal disease. In some embodiment, the GREM1-related disease or condition is kidney disease.

### GREM1 Antagonist

In some embodiment, the GREM1 antagonist reduces GREM1 level or GREM1 activity. For example, the GREM1 antagonist can partially inhibit, i.e., reduce the expression and/or activity of GREM1, or completely inhibit, i.e., completely eliminate the expression and/or activity of GREM1.

The GREM1 antagonist may reduce GREM1 level or activity by at least 10%, at least 20%, at least 30% at least 40%, at least 50%, at least 60%, at least 70%, at least 80%, at least 90% or at least 95%.

Any function or activity of GREM1 can be reduced. In certain embodiments, the GREM1 antagonist reduces GREM1-mediated inhibition on BMP signaling and/or GREM1-mediated activation of MAPK signaling, optionally in a cancer cell. In some embodiments, the GREM1 antagonist inhibits BMP non-dependent GREM1 activity.

In certain embodiments, the GREM1 antagonist selectively reduces the function or activity GREM1 in cancer cell over non-cancer cell. The reduction of function or activity or level of GREM1 can be measured using any suitable assay performed in the presence and absence of the GREM1 antagonist.

In some embodiment, the GREM1 antagonist comprises a GREM1-FGFR1 axis inhibitor. The present disclosure surprisingly found that GREM1 appears to play a role in the activation of MAPK signaling, which may be independent of BMP, and possibly acts as a novel ligand of FGFR. Therefore, a GREM1-FGFR1 axis inhibitor provided herein refers to any inhibitor that can interfere with or inhibit the signaling of GREM1 dependent FGFR1 signaling, or blocks binding between GREM1 and FGFR1.

In some embodiments, the GREM1-FGFR1 axis inhibitor comprises an FGFR1-binding inhibitor.

In some embodiments, the FGFR1-binding inhibitor binds to extracellular domain 2 of FGFR1, and optionally binds to FGFR1 at an epitope comprising residue Glu 160, wherein residue number is according to SEQ ID NO: 75.

In some embodiments, the GREM1-FGFR1 axis inhibitor binds to hGREM1 at an epitope comprising residue Lys 123 and/or residue Lys 124, wherein residue number is according to SEQ ID NO: 69; or blocks FGFR1 binding to the residue Lys 123 and/or residue Lys 124 of FGFR1.

In some embodiments, the GREM1 antagonist or GREM1-FGFR1 axis inhibitor comprises an antibody against hGREM1 or an antigen-binding fragment thereof provided herein.

In various embodiments, the GREM1 antagonist may be an anti-GREM1 antibody or antigen-binding fragment thereof, a GREM1 mimetic peptide, a nucleic acid targeting gremlin RNA or DNA, a compound inhibiting interaction between gremlin and BMP, or a compound inhibiting GREM1 mediated biological activity. The GREM1 antagonist can comprise an anti-GREM1 antibody or antigen-binding fragment thereof, an inhibitory GREM1 mimetic peptide, an inhibitory nucleic acid targeting GREM1 RNA or DNA, a compound inhibiting interaction between gremlin and BMP, a polynucleotide encoding the inhibitory nucleic acid, a compound inhibiting the GREM1 activity.

In some embodiment, the inhibitory nucleic acid targeting GREM1 RNA or DNA comprises a short hairpin RNA (shRNA), micro interfering RNA (miRNA), double strand RNA (dsRNA), small interfering RNA (siRNA), guide RNA, or antisense oligonucleotide.

In certain embodiments, the nucleic acid targeting gremlin RNA or DNA is a non-coding nucleic acid, for example, short hairpin RNA (shRNA), micro interfering RNA (miRNA), double strand RNA (dsRNA), small interfering RNA (siRNA), guide RNA, antisense oligonucleotide, or the polynucleotide encoding such.

In certain embodiments, the GREM1 antagonist may reduce level of GREM1 at mRNA level or protein level. For example, the GREM1 antagonist may promote degradation of GREM1 at mRNA level or protein level, disrupt DNA encoding GREM1, or reduce transcription from the DNA encoding GREM1. Such GREM1 antagonist can include a non-coding nucleic acid targeting GREM1 mRNA or DNA, for example, short hairpin RNA (shRNA), micro interfering RNA (miRNA), double strand RNA (dsRNA), small interfering RNA (siRNA), guide RNA, antisense oligonucleotide, and the polynucleotide encoding such. The GREM1 antagonist can also include agents that promotes degradation of GREM1 protein.

In certain embodiments, the GREM1 antagonist may be an agent interfering with (e.g. reducing) GREM1 binding to BMP, such as BMP2/4/7. For example, the GREM1 antagonist may be an anti-GREM1 antibody, a GREM1 mimetic peptide, or a chemical compound that reduces or blocks binding of GREM1 to BMP, thereby reduces GREM1-mediated inhibition on BMP signaling. A GREM1 antagonist may compete with GREM1 for binding to BMP, but it may also bind to a different epitope or binding site that does not directly affects GREM1 binding to BMP but still reduces its biological function mediated by GREM1.

In certain embodiments, the GREM1 antagonist comprises an anti-GREM1 antibody, a compound inhibiting interaction between GREM1 and BMP, or a compound inhibiting GREM1 mediated biological activity (e.g. activation of MAPK signaling, or inhibition on BMP signaling).

In certain embodiments, the GREM1 antagonist can comprise any of anti-GREM1 antibodies provided herein, or any existing anti-GREM1 antibodies such as those disclosed, for example, in WO2018/115017, WO2019/158658, WO2019/243801, WO2014159010, disclosure of which are hereby incorporated by reference in their entirety.

### GREM1 Antibody

In certain embodiments, the GREM1 antagonist comprises an antibody against human gremlin1 (hGREM1) or an antigen-binding fragment thereof that binds to a different epitope than other anti-GREM1 antibodies. For example, the antibody against human gremlin1 (hGREM1) or an antigen-binding fragment thereof used as the GREM1 antagonist does not bind to a BMP-binding loop comprising an amino acid sequence of SEQ ID NO: 63.

In certain embodiments, the GREM1 antagonist comprises an antibody against human gremlin1 (hGREM1) or an antigen-binding fragment thereof, comprising a heavy chain variable (VH) region and/or a light chain variable (VL) region, wherein the heavy chain variable region is selected from the group consisting of:
a) a heavy chain variable region comprising a HCDR1 comprising the sequence of SEQ ID NO: 1, a HCDR2 comprising the sequence of SEQ ID NO: 2, and a HCDR3 comprising the sequence of SEQ ID NO: 3;
b) a heavy chain variable region comprising a HCDR1 comprising the sequence of SEQ ID NO: 11, a HCDR2 comprising the sequence of SEQ ID NO: 12, and a HCDR3 comprising the sequence of SEQ ID NO: 13;
c) a heavy chain variable region comprising a HCDR1 comprising the sequence of SEQ ID NO: 21, a HCDR2 comprising the sequence of SEQ ID NO: 22, and a HCDR3 comprising the sequence of SEQ ID NO: 23; and
d) a heavy chain variable region comprising a HCDR1 comprising the sequence of SEQ ID NO: 31, a HCDR2 comprising the sequence of SEQ ID NO: 32, and a HCDR3 comprising the sequence of SEQ ID NO: 33.

In certain embodiments, the GREM1 antagonist comprises an antibody against human gremlin1 (hGREM1) or an antigen-binding fragment thereof, comprising a heavy chain variable (VH) region and/or a light chain variable (VL) region, wherein the light chain variable region is selected from the group consisting of:
a) a light chain variable region comprising a LCDR1 comprising the sequence of SEQ ID NO: 4, a LCDR2 comprising the sequence of SEQ ID NO: 5, and a LCDR3 comprising the sequence of SEQ ID NO: 6;
b) a light chain variable region comprising a LCDR1 comprising the sequence of SEQ ID NO: 14, a LCDR2 comprising the sequence of SEQ ID NO: 15, and a LCDR3 comprising the sequence of SEQ ID NO: 16;
c) a light chain variable region comprising a LCDR1 comprising the sequence of SEQ ID NO: 24, a LCDR2 comprising the sequence of SEQ ID NO: 25, and a LCDR3 comprising the sequence of SEQ ID NO: 26; and
d) a light chain variable region comprising a LCDR1 comprising the sequence of SEQ ID NO: 34, a LCDR2 comprising the sequence of SEQ ID NO: 35, and a LCDR3 comprising the sequence of SEQ ID NO: 36.

In certain embodiments, the GREM1 antagonist comprises an antibody against human gremlin1 (hGREM1) or an antigen-binding fragment thereof, comprising a heavy chain variable (VH) region and/or a light chain variable (VL) region, wherein:
a) the heavy chain variable region comprises a HCDR1 comprising the sequence of SEQ ID NO: 1, a HCDR2 comprising the sequence of SEQ ID NO: 2, and a HCDR3 comprising the sequence of SEQ ID NO: 3; and the light chain variable region comprises a LCDR1 comprising the sequence of SEQ ID NO: 4, a LCDR2 comprising the sequence of SEQ ID NO: 5, and a LCDR3 comprising the sequence of SEQ ID NO: 6;
b) the heavy chain variable region comprises a HCDR1 comprising the sequence of SEQ ID NO: 11, a HCDR2 comprising the sequence of SEQ ID NO: 12, and a HCDR3 comprising the sequence of SEQ ID NO: 13; and the light chain variable region comprises a LCDR1 comprising the sequence of SEQ ID NO: 14, a LCDR2 comprising the sequence of SEQ ID NO: 15, and a LCDR3 comprising the sequence of SEQ ID NO: 16;
c) the heavy chain variable region comprises a HCDR1 comprising the sequence of SEQ ID NO: 21, a HCDR2 comprising the sequence of SEQ ID NO: 22, and a HCDR3 comprising the sequence of SEQ ID NO: 23; and the light chain variable region comprises a LCDR1 comprising the sequence of SEQ ID NO: 24, a LCDR2 comprising the sequence of SEQ ID NO: 25, and a LCDR3 comprising the sequence of SEQ ID NO: 26; or
d) the heavy chain variable region comprises a HCDR1 comprising the sequence of SEQ ID NO: 31, a HCDR2 comprising the sequence of SEQ ID NO: 32, and a HCDR3 comprising the sequence of SEQ ID NO: 33; and the light chain variable region comprises a LCDR1 comprising the sequence of SEQ ID NO: 34, a LCDR2 comprising the sequence of SEQ ID NO: 35, and a LCDR3 comprising the sequence of SEQ ID NO: 36.

In certain embodiments, the GREM1 antagonist comprises an antibody against human gremlin1 (hGREM1) or an antigen-binding fragment thereof, comprising a heavy chain variable (VH) region and/or a light chain variable (VL) region, wherein the heavy chain variable region comprises a sequence selected from the group consisting of SEQ ID NO: 7, SEQ ID NO: 17, SEQ ID NO: 27, SEQ ID NO: 37, SEQ ID NO: 41, SEQ ID NO: 43, SEQ ID NO: 45, SEQ ID NO: 51, SEQ ID NO: 53, SEQ ID NO: 55 and SEQ ID NO: 57, and a homologous sequence thereof having at least 80% sequence identity yet retaining specific binding specificity or affinity to gremlin.

In certain embodiments, the GREM1 antagonist comprises an antibody against human gremlin1 (hGREM1) or an antigen-binding fragment thereof, comprising a heavy chain variable (VH) region and/or a light chain variable (VL) region, wherein the light chain variable region comprises a sequence selected from the group consisting of SEQ ID NO: 8, SEQ ID NO: 18, SEQ ID NO: 28, SEQ ID NO: 38, SEQ ID NO: 47, SEQ ID NO: 49, SEQ ID NO: 59 and SEQ ID NO: 61, and a homologous sequence thereof having at least 80% sequence identity yet retaining specific binding specificity or affinity to gremlin.

In certain embodiments, the GREM1 antagonist comprises:
a) a heavy chain variable region comprising the sequence of SEQ ID NO: 7 and a light chain variable region comprising the sequence of SEQ ID NO: 8; or
b) a heavy chain variable region comprising a sequence of SEQ ID NO: 17 and a light chain variable region comprising a sequence of SEQ ID NO: 18; or
c) a heavy chain variable region comprising a sequence of SEQ ID NO: 27 and a light chain variable region comprising a sequence of SEQ ID NO: 28; or
d) a heavy chain variable region comprising a sequence of SEQ ID NO: 37 and a light chain variable region comprising a sequence of SEQ ID NO: 38; or
e) a heavy chain variable region comprising a sequence selected from the group consisting of SEQ ID NO: 41, SEQ ID NO: 43 and SEQ ID NO: 45, and a light chain variable region comprising a sequence selected from the group consisting of SEQ ID NO: 47 and SEQ ID NO: 49; or
f) a pair of heavy chain variable region and light chain variable region sequences selected from the group consisting of: SEQ ID NOs: 41/47, 41/49, 43/47, 43/49, 45/47, and 45/49; or
g) a heavy chain variable region comprising a sequence selected from the group consisting of SEQ ID NO: 51, SEQ ID NO: 53, SEQ ID NO: 55 and SEQ ID NO: 57, and a light chain variable region comprising a sequence selected from the group consisting of SEQ ID NO: 59 and SEQ ID NO: 61; or
h) a pair of heavy chain variable region and light chain variable region sequences selected from the group consisting of: SEQ ID NOs: 51/59, 51/61, 53/59, 53/61, 55/59, 55/61, 57/59, and 57/61.

In certain embodiments, the antibodies provided herein comprise one or more (e.g. 1, 2, 3, 4, 5, or 6) CDR sequences of anti-hGREM1 antibodies 14E3, 69H5, 22F1, 56C11.

“14E3” as used herein refers to a mouse antibody having a heavy chain variable region of SEQ ID NO: 7, and a light chain variable region of SEQ ID NO: 8.

“69H5” as used herein refers to a mouse antibody having a heavy chain variable region of SEQ ID NO: 27, and a light chain variable region of SEQ ID NO: 28.

“22F1” as used herein refers to a mouse antibody having a heavy chain variable region of SEQ ID NO: 17, and a light chain variable region of SEQ ID NO: 18.

“56C11” as used herein refers to a mouse antibody having a heavy chain variable region of SEQ ID NO: 37, and a light chain variable region of SEQ ID NO: 38.

Table 1 shows the CDR sequences of these anti-hGREM1 antibodies. The heavy chain and light chain variable region sequences are also provided below in Table 2.

TABLE 1
Sequences of anti-hGREM1 antibodies' CDR region

| Antibody | Region | CDR1 | CDR2 | CDR3 |
|---|---|---|---|---|
| 14E3 | HCDR | SEQ ID NO: 1 | SEQ ID NO: 2 | SEQ ID NO: 3 |
| | | TYGMA | WINTLSGEPTYAD | EPMDY |
| | | | DFKG | |
| | LCDR | SEQ ID NO: 4 | SEQ ID NO: 5 | SEQ ID NO: 6 |
| | | KSSQSLLDSDGK | LVSKLDS | WQGAHFPLT |
| | | TYLS | | |
| 22F1 | HCDR | SEQ ID NO: 11 | SEQ ID NO: 12 | SEQ ID NO: 13 |
| | | DYYMN | DINPKDGDSGYSH | GFTTVVARGDY |
| | | | KFKG | |
| | LCDR | SEQ ID NO: 14 | SEQ ID NO: 15 | SEQ ID NO: 16 |
| | | KSSQSLLDSDGKT | LVSKLDS | WQGTHFPYT |
| | | YLN | | |
| 69H5 | HCDR | SEQ ID NO: 21 | SEQ ID NO: 22 | SEQ ID NO: 23 |
| | | DDYMH | WIDPENGDTEYAS | WATVPDFDY |
| | | | KFQG | |
| | LCDR | SEQ ID NO: 24 | SEQ ID NO: 25 | SEQ ID NO: 26 |
| | | KSSQSLLNRSNQK | FTSTRES | QQHYSTPFT |
| | | NYLA | | |
| 56C11 | HCDR | SEQ ID NO: 31 | SEQ ID NO: 32 | DPIYYDYDEVA |
| | | DFYMN | DINPNNGGTSYNQ | SEQ ID NO: 33 |
| | | | KFKG | Y |
| | LCDR | SEQ ID NO: 34 | SEQ ID NO: 35 | SEQ ID NO: 36 |
| | | RSSQSLVHSNGNT | KVSNRFS | SQSTHVPLT |
| | | YLH | | |

TABLE 2
Sequences of mouse antibody VH/VL

| | VH | VL |
|---|---|---|
| 14E3 | SEQ ID NO: 7 | SEQ ID NO: 8 |
| | QIQLVQSGPELKKPGETVKISCKTS | DVVMTQTPLTLSITIGQPASISCKS |
| | GSTFTTYGMAWMKQAPGKGLTW | SQSLLDSDGKTYLSWLLQRPDQS |
| | MGWINTLSGEPTYADDFKGRFAFS | PKRLISLVSKLDSGVPDRITGSGS |
| | LKTSANTAYLQINNLKNEDAATYF | GTDFTLKISRVEAEDLGIYYCWQ |
| | CAREPMDYWGQGTSVIVSS | GAHFPLTFGAGTKLELK |
| 22F1 | SEQ ID NO: 17 | SEQ ID NO: 18 |
| | EAQLQQSGPELVKPGASVKISCKA | DVVMTQTPLTLSVTIGQPASISCK |
| | SGYSFTDYYMNWLKQSHGKSLE | SSQSLLDSDGKTYLNWLLQRPG |
| | WIGDINPKDGDSGYSHKFKGKAT | QSPKRLIYLVSKLDSGFPDRFTGS |
| | LTVDKSSSTAYMELRSLTSEDSAV | GSGTDFTLKISRVEAEDLGVYYC |
| | YYCASGFTTVVARGDYWGQGTTL | WQGTHFPYTFGGGTKLEIK |
| | TVSS | |
| 69H5 | SEQ ID NO: 27 | SEQ ID NO: 28 |
| | EVQLQQSGAELVRPGASVKLSCT | DIVMTQSPSSLAMSVGQKVTMS |
| | ASGFNIKDDYMHWVKRRPEQGL | CKSSQSLLNRSNQKNYLAWYQQ |
| | EWIGWIDPENGDTEYASKFQGKA | KPGQSPKLLVHFTSTRESGVPDR |
| | TITADTSSNTAYLQLSSLTSEDTAV | FIGSGSGTDFTLTISNLQAEDLAD |
| | YYCTTWATVPDFDYWGQGTTLTV | YFCQQHYSTPFTFGSGTKLEIK |
| | SS | |
| 56C11 | SEQ ID NO: 37 | SEQ ID NO: 38 |
| | EVQLQQSGPELVKPGASVKISCKA | DVVMTQTPLSLPVSLGDQASISC |
| | SGYTFTDFYMNWVKQSHGKSLE | RSSQSLVHSNGNTYLHWYLQKP |
| | WIGDINPNNGGTSYNQKFKGKAT | GQSPKLLIYKVSNRFSGVPDRFS |
| | LTVDKSSSTAYMELRSLTSEDSAV | GSGSGTDFTLKISRVEAEDLGVY |
| | YYCARDPIYYDYDEVAYWGQGTL | FCSQSTHVPLTFGAGTKLELK |
| | VTVSA | |

The anti-hGREM1 antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

TABLE 3
Sequences of humanized 14E3 (Hu14E3) and humanized
22F1 (Hu14E3)

| Antibody chain | Sequences |
|---|---|
| Hu14E3-Ha VH | QVQLVQSGSELKKPGASVKVSCKASGYTFTTYGMAWMR |
| | QAPGQGLEWMGWINTLSGEPTYADDFKGRFVFSLDTSVS |
| | TAYLQISSLKAEDTAVYYCAREPMDYWGQGTMVTVSS |
| | (SEQ ID NO: 41) |
| Hu14E3-Hb VH | QIQLVQSGSELKKPGASVKVSCKASGYTFTTYGMAWMRQ |
| | APGQGLEWMGWINTLSGEPTYADDFKGRFAFSLDTSVST |
| | AYLQISSLKAEDTAVYYCAREPMDYWGQGTMVTVSS |
| | (SEQ ID NO: 43) |
| Hu14E3-Hc VH | QIQLVQSGSELKKPGASVKVSCKASGSTFTTYGMAWMKQ |
| | APGQGLTWMGWINTLSGEPTYADDFKGRFAFSLDTSVST |
| | AYLQISSLKAEDTAVYYCAREPMDYWGQGTMVTVSS |
| | (SEQ ID NO: 45) |
| Hu14E3-La VL | DVVMTQSPLSLPVTLGQPASISCKSSQSLLDSDGKTYLSW |
| | LQQRPGQSPRRLIYLVSKLDSGVPDRFSGSGSGTDFTLKIS |
| | RVEAEDVGVYYCWQGAHFPLTFGQGTKLEIK (SEQ ID |
| | NO: 47) |
| Hu14E3-Lb VL | DVVMTQSPLSLPVTLGQPASISCKSSQSLLDSDGKTYLSW |
| | LQQRPGQSPRRLISLVSKLDSGVPDRFSGSGSGTDFTLKISR |
| | VEAEDVGVYYCWQGAHFPLTFGQGTKLEIK (SEQ ID NO: |
| | 49) |
| Hu22F1-Ha VH | QVQLVQSGAEVKKPGASVKVSCKASGYSFTDYYMNWVR |
| | QAPGQGLEWMGDINPKDGDSGYSHKFKGRVTMTRDTSTS |
| | TVYMELSSLRSEDTAVYYCASGFTTVVARGDYWGQGTT |
| | VTVSS (SEQ ID NO: 51) |
| Hu22F1-Hb VH | QVQLVQSGAEVKKPGASVKVSCKASGYSFTDYYMNWVR |
| | QAPGQGLEWMGDINPKDGDSGYSHKFKGRVTMTVDKST |
| | STVYMELSSLRSEDTAVYYCASGFTTVVARGDYWGQGTT |
| | VTVSS (SEQ ID NO: 53) |
| Hu22F1-Hc VH | QAQLVQSGAEVKKPGASVKVSCKASGYSFTDYYMNWVR |
| | QAPGQGLEWMGDINPKDGDSGYSHKFKGRVTLTVDKSTS |
| | TVYMELRSLRSEDTAVYYCASGFTTVVARGDYWGQGTT |
| | VTVSS (SEQ ID NO: 55) |
| Hu22F1-Hd VH | QAQLVQSGAEVKKPGASVKVSCKASGYSFTDYYMNWLR |
| | QAPGQGLEWIGDINPKDGDSGYSHKFKGRATLTVDKSTST |
| | VYMELRSLRSEDTAVYYCASGFTTVVARGDYWGQGTTV |
| | TVSS (SEQ ID NO: 57) |
| Hu22F1-La VL | DVVMTQSPLSLPVTLGQPASISCKSSQSLLDSDGKTYLNW |
| | LQQRPGQSPRRLIYLVSKLDSGVPDRFSGSGSGTDFTLKIS |
| | RVEAEDVGVYYCWQGTHFPYTFGGGTKVEIK (SEQ ID |
| | NO: 59) |
| Hu22F1-Lb VL | DVVMTQSPLSLPVTLGQPASISCKSSQSLLDSDGKTYLNW |
| | LQQRPGQSPRRLIYLVSKLDSGFPDRFSGSGSGTDFTLKISR |
| | VEAEDVGVYYCWQGTHFPYTFGGGTKVEIK (SEQ ID NO: |
| | 61) |

In certain embodiments, the GREM1 antagonist comprises an anti-human GREM1 antibody or antigen-binding fragment thereof, which is: a) capable of binding to hGREM1 at an epitope comprising residue Gln27 and/or residue Asn33, wherein residue number is according to SEQ ID NO: 69, and/or b) capable of binding to a hGREM1 fragment comprising residue Gln27 and/or residue Asn33, optionally the hGREM1 fragment has a length of at least 3 (e.g. 4, 5, 6, 7, 8, 9, or 10) amino acid residues; and/or c) capable of reducing hGREM1-mediated inhibition on BMP signaling selectively in a cancer cell over a non-cancer cell; and/or d) exhibiting no more than 50% reduction of hGREM1-mediated inhibition on BMP signaling in a non-cancer cell; and/or e) capable of binding to a chimeric hGREM1 comprising an amino acid sequence of SEQ ID NO: 68, and/or f) capable of reducing hGREM1-mediated activation on MAPK signaling, and/or g) capable of binding to hGREM1 at a K_{D} of no more than 1 nM as measured by Fortebio.

In certain embodiments, the anti-GREM1 antibody or antigen-binding fragment thereof further comprising one or more amino acid residue substitutions or modifications yet retains specific binding specificity or affinity to hGREM1.

In certain embodiments, at least one of the substitutions or modifications is in one or more of the CDR sequences, and/or in one or more of the non-CDR regions of the VH or VL sequences.

In certain embodiments, the anti-GREM1 antibody or antigen-binding fragment thereof further comprising an immunoglobulin constant region, optionally a constant region of human Ig, or optionally a constant region of human IgG.

In certain embodiments, the constant region comprises a constant region of human IgG1, IgG2, IgG3, or IgG4.

In certain embodiments, the anti-GREM1 antibody or antigen-binding fragment thereof is humanized.

In certain embodiments, the anti-GREM1 antibody or antigen-binding fragment thereof is a diabody, a Fab, a Fab′, a F(ab′)2, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

In certain embodiments, the anti-GREM1 antibody or antigen-binding fragment thereof is bispecific. The term “bispecific” as used herein encompasses molecules having more than two specificity and molecules having more than two specificity, i.e. multispecific. In certain embodiments, the bispecific antibodies and antigen-binding fragments thereof provided herein is capable of specifically binding to a first and a second epitopes of hGREM1, or capable of specifically binding to hGREM1 and a second antigen. In certain embodiments, the first epitope and the second epitopes of hGREM1 are distinct from each other or non-overlapping. In certain embodiments, the bispecific antibodies and antigen-binding fragments thereof can bind to both the first epitope and the second epitope at the same time.

In certain embodiments, the second antigen is different from hGREM1. In certain embodiments, the second antigen comprises an immune related target. In certain embodiments, the second antigen comprises PD-1, PD-L1, PD-L2, CTLA-4, TIM-3, LAG3, A2AR, CD160, 2B4, TGF B, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 or CD83.

In certain embodiments, the tumor antigen comprises a tumor specific antigen or a tumor associated antigen. In certain embodiments, the tumor antigen comprises prostate specific antigen (PSA), CA-125, gangliosides G(D2), G(M2) and G(D3), CD20, CD52, CD33, Ep-CAM, CEA, bombesin-like peptides, HER2/neu, epidermal growth factor receptor (EGFR), erbB2, erbB3/HER3, erbB4, CD44v6, Ki-67, cancer-associated mucin, VEGF, VEGFRs (e.g., VEGFR-1, VEGFR-2, VEGFR-3), estrogen receptors, Lewis-Y antigen, TGFß1, IGF-1 receptor, EGFa, c-Kit receptor, transferrin receptor, Claudin 18.2, GPC-3, Nectin-4, ROR1, methothelin, PCMA, MAGE-1, MAGE-3, BAGE, GAGE-1, GAGE-2, p15, BCR-ABL, E2APRL, H4-RET, IGH-IGK, MYL-RAR, IL-2R, CO17-1A, TROP2, or LIV-1.

In certain embodiments, the anti-GREM1 antibody or antigen-binding fragment thereof is not cross-reactive to mouse gremlin1.

In certain embodiments, the anti-GREM1 antibody or antigen-binding fragment thereof is cross-reactive to mouse gremlin1.

In certain embodiments, the GREM1 antagonist is capable of reducing GREM1-mediated activation on MAPK signaling.

### Combination Treatment Methods

The treatment methods provided herein can further comprise a step of providing a second therapeutic agent and a step of administering a therapeutically effective amount of the second therapeutic agent to the subject, thereby treating, preventing, reducing the severity of and/or slowing the progression of the GREM1-related disease or condition in the subject. In certain of these embodiments, the GREM1-related disease or condition can be characterized in deficiency of PTEN and/or p53, and/or is a cancer which is characterized in reduced androgen receptor (AR) signaling.

In certain of these embodiments, the GREM1 antagonist as disclosed herein that is administered in combination with one or more additional therapeutic agents may be administered simultaneously with the one or more additional therapeutic agents, and in certain of these embodiments the GREM1 antagonist and the additional therapeutic agent(s) may be administered as part of the same pharmaceutical composition. However, a GREM1 antagonist administered “in combination” with another therapeutic agent does not have to be administered simultaneously with or in the same composition as the agent. A GREM1 antagonist administered prior to or after another agent is considered to be administered “in combination” with that agent as the phrase is used herein, even if the GREM1 antagonist and second agent are administered via different routes. Where possible, additional therapeutic agents administered in combination with the GREM1 antagonist disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

### i). Combinatory Treatment for Cancers

In some embodiments, the GREM1 antagonist disclosed herein may be administered for treating cancer in combination with a second anti-cancer drug, for example, a chemotherapeutic agent (e.g., Cisplatin), an anti-cancer drug, radiation therapy, an immunotherapy (e.g., an immune checkpoint inhibitor, MPDL-3280A), anti-angiogenesis agent, a targeted therapy, a cellular therapy, a gene therapy agent, a hormonal therapy agent, cytokines, palliative care, surgery for the treatment of cancer (e.g., tumorectomy), one or more anti-emetics, treatments for complications arising from chemotherapy, or a diet supplement for cancer patients.

The term “immunotherapy” as used herein, refers to a type of that stimulates immune system to fight against disease such as cancer or that boosts immune system in a general way. Immunotherapy includes passive immunotherapy by delivering agents with established tumor-immune reactivity (such as effector cells) that can directly or indirectly mediate anti-tumor effects and does not necessarily depend on an intact host immune system (such as an antibody therapy or CAR-T cell therapy). Immunotherapy can further include active immunotherapy, in which treatment relies on the in vivo stimulation of the endogenous host immune system to react against diseased cells with the administration of immune response-modifying agents.

Examples of immunotherapy include, without limitation, checkpoint modulators, adoptive cell transfer, cytokines, oncolytic virus and therapeutic vaccines.

Checkpoint modulators can interfere with the ability of cancer cells to avoid immune system attack, and help the immune system respond more strongly to a tumor. Immune checkpoint molecule can mediate co-stimulatory signal to augment immune response, or can mediate co-inhibitory signals to suppress immune response. Examples of checkpoint modulators include, without limitation, modulators of PD-1, PD-L1, PD-L2, CTLA-4, TIM-3, LAG3, A2AR, CD160, 2B4, TGF B, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 and CD83. In certain embodiments, the immune checkpoint modulator comprises a PD-1/PD-L1 axis inhibitor.

Adoptive cell transfer, which is a treatment that attempts to boost the natural ability of the T cells to fight cancer. In this treatment, T cells are taken from the patient, and are expanded and activated in vitro. In certain embodiments, the T cells are modified in vitro to CAR-T cells. T cells or CAR-T cells that are most active against the cancer are cultured in large batches in vitro for 2 to 8 weeks. During this period, the patients will receive treatments such as chemotherapy and radiation therapy to reduce the body's immunity. After these treatments, the in vitro cultured T cells or CAR-T cells will be given back to the patient. In certain embodiments, the immunotherapy is CAR-T therapy.

Cytokine therapy can also be used to enhance tumor antigen presentation to the immune system. The two main types of cytokines used to treat cancer are interferons and interleukins. Examples of cytokine therapy include, without limitation, interferons such as interferon-α, -ß, and —Y, colony stimulating factors such as macrophage-CSF, granulocyte macrophage CSF, and granulocyte-CSF, insulin growth factor (IGF-1), vascular endothelial growth factor (VEGF), transforming growth factor (TGF), fibroblast growth factor (FGF), interleukins such as IL-1, IL-1a, IL-2, IL-3, IL-4, IL-5, IL-6, IL-7, IL-8, IL-9, IL-10, IL-11, and IL-12, tumor necrosis factors such as TNF-α and TNF-β or any combination thereof.

Oncolytic virus are genetically modified virus that can kill cancer cells. Oncolytic virus can specifically infect tumor cells, thereby leading to tumor cell lysis followed by release of large amount of tumor antigens that trigger the immune system to target and eliminate cancer cells having such tumor antigens. Examples of oncolytic virus include, without limitation, talimogene laherparepvec.

Therapeutic vaccines work against cancer by boosting the immune system's response to cancer cells. Therapeutic vaccines can comprise non-pathogenic microorganism (e.g. Mycobacterium bovis Bacillus Calmette-Guérin, BCG), genetically modified virus targeting a tumor cell, or one or more immunogenic components. For example, BCG can be inserted directly into the bladder with a catheter and can cause an immune response against bladder cancer cells.

Anti-angiogenesis agent can block the growth of blood vessels that support tumor growth. Some of the anti-angiogenesis agent target VEGF or its receptor VEGFR. Examples of anti-angiogenesis agent include, without limitation, Axitinib, Bevacizumab, Cabozantinib, Everolimus, Lenalidomide, Lenvatinib mesylate, Pazopanib, Ramucirumab, Regorafenib, Sorafenib, Sunitinib, Thalidomide, Vandetanib, and Ziv-aflibercept.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

Targeted therapy can target, for example, tyrosine kinase receptors and nuclear receptors. Examples of such receptors include, erbB1 (EGFR or HER1), erbB2 (HER2), erbB3, erbB4, FGFR, platelet-derived growth factor receptor (PDGFR), and insulin-like growth factor-1 receptor (IGF-1R), androgen receptors (ARs), estrogen receptors (ERs), nuclear receptors (NR) and PRs.

Targeted therapy can target molecules in tyrosine kinase or nuclear receptors signaling cascade, such as, Erk and PI3K/Akt, AP-2a, AP-2B, AP-2y, mitogen-activated protein kinase (MAPK), PTEN, p53, p19ARF, Rb, Apaf-1, CD-95/Fas, TRAIL-R1/R2, Caspase-8, Forkhead, Box 03A, MDM2, IAPs, NF-kB, Myc, P13K, Ras, FLIP, heregulin (HRG) (also known as gp30), Bcl-2, Bcl-xL, Bax, Bak, Bad, Bok, Bik, Blk, Hrk, BNIP3, BimL, Bid, and EGL-1.

Targeted therapy can also target tumor-associated ligands such estrogen, estradiol (E2), progesterone, oestrogen, androgen, glucocorticoid, prolactin, thyroid hormone, insulin, P70 S6 kinase protein (PS6), Survivin, fibroblast growth factors (FGFs), EGF, Neu Differentiation Factor (NDF), transforming growth factor alpha (TGF-α), IL-1A, TGF-beta, IGF-1, IGF-II, IGFBPs, IGFBP proteases, and IL-10.

In some embodiments, the GREM1 antagonist disclosed herein may be administered for treating prostate cancer in combination with a second anti-cancer drug. In certain embodiments, the anti-cancer drug comprises an anti-prostate cancer drug. In some embodiments, the anti-prostate cancer drug comprises an androgen axis inhibitor; an androgen synthesis inhibitor; an ADP-ribose polymerase (PARP) inhibitor; or a combination thereof.

In certain embodiments, the androgen axis inhibitor is selected from the group consisting of Luteinizing hormone-releasing hormone (LHRH) agonists, LHRH antagonists and androgen receptor antagonist.

In certain embodiments, the androgen axis inhibitor is degarelix, bicalutamide, flutamide, nilutamide, apalutamide, darolutamide, enzalutamide, or abiraterone.

In certain embodiments, the androgen synthesis inhibitor is abiraterone acetate or ketoconazole.

In certain embodiments, the PARP inhibitor is olaparib, or rucaparib.

In certain embodiments, the anti-prostate cancer drug is selected from the group consisting of Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate) and Zytiga (Abiraterone Acetate).

In certain embodiments, the diet supplement for cancer patients can be a suitable supplement that has a protective effect against cancer. In certain embodiments, the diet supplement comprises indole-3-carbinol or comprises a derivative thereof that gives rise to indole-3-carbinol after ingestion. Indole-3-carbinol is believed to have protective effects against cancer and also may be preventative against precancerous conditions.

In certain embodiments, the antibodies or antigen-binding fragments disclosed herein may be administered in combination with indole-3-carbinol or a derivative thereof that gives rise to indole-3-carbinol after ingestion. In certain embodiments, such combination is useful for treating gremlin-related diseases. In certain embodiments, such combination is useful for treating cancer, for example, breast cancer, hepatocellular carcinoma, and colorectal cancer. In certain embodiments, such combination is useful for treating breast cancer, for example, triple negative breast cancer.

### ii) Combinatory Treatment for Non-Cancer Diseases

In some embodiments, the second therapeutic agent may be administered to manage or treat at least one complication associated with non-cancer disease (e.g., fibrosis) or cancer.

In certain embodiments, the second therapeutic agent is anti-fibrotic agent such as pirfenidone, an anti-inflammatory drug, a NSAID, a corticosteroid such as prednisone, a nutritional supplement, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab)], an antibody to a cytokine such as IL-1, IL-6, IL-13, IL-4, IL-17, IL-25, IL-33 or TGF-ß, and any other palliative therapy useful for ameliorating at least one symptom associated with a fibrosis-associated condition or cancer. In certain embodiment, the second therapeutic agent is anti-integrin inhibitor.

In another aspect, the present disclosure provides kits or pharmaceutical compositions comprising the GREM1 antagonist provided herein and the second therapeutic agent, which may be formulated in one composition, or in different compositions. An instructions for use or indications can be further included to provide information on how combined therapy are to be carried out.

In certain embodiments, the diet supplement for cancer patients can be a suitable supplement that has a protective effect against cancer. In certain embodiments, the diet supplement comprises indole-3-carbinol or comprises a derivative thereof that gives rise to indole-3-carbinol after ingestion. Indole-3-carbinol is believed to have protective effects against cancer and also may be preventative against precancerous conditions.

In certain embodiments, the antibodies or antigen-binding fragments disclosed herein may be administered in combination with indole-3-carbinol or a derivative thereof that gives rise to indole-3-carbinol after ingestion. In certain embodiments, such combination is useful for treating gremlin-related diseases. In certain embodiments, such combination is useful for treating cancer, for example, breast cancer, hepatocellular carcinoma, and colorectal cancer. In certain embodiments, such combination is useful for treating breast cancer, for example, triple negative breast cancer.

### Administration Route and Dosage Regime

The GREM1 antagonist as provided herein may be administered at a therapeutically effective dosage. The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the GREM1 antagonist (e.g. the antibody or antigen-binding fragment) as provided herein may be administered at a therapeutically effective dosage of about 0.01 mg/kg to about 100 mg/kg. In certain embodiments, the administration dosage may change over the course of treatment. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The GREM1 antagonist (e.g. antibodies and antigen-binding fragments) disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

Methods of Detection and/or Diagnosis

### AR Expression or Signaling Determination

In one aspect, the present disclosure provides a method of determining likelihood of responsiveness to a GREM1 antagonist in a subject having or suspected of having cancer, comprising: (a) detecting androgen receptor (AR) expression or signaling in a biological sample from the subject, and (b) determining the likelihood of responsiveness based on the AR expression or signaling detected in step (a).

As used in the present disclosure, the term “likelihood” and “likely” denotes a chance in percent of how probable a therapeutic response is to occur. In some embodiments, a subject with a disease or condition (e.g. cancer) identified as “likely to respond” refers to a subject with a disease or condition who has more than 30% chance, more than 40% chance, more than 50% chance, more than 60% chance, more than 70% chance, more than 80% chance, more than 90% chance of responding to the treatment with a GREM1 antagonist as provided herein.

In a patient, beneficial response can be expressed in terms of a number of clinical parameters, including loss of detectable tumor (complete response), decrease in tumor size and/or tumor cell number (partial response), tumor growth arrest (stable disease), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment.

AR expression can be detected using any suitable methods known in the art. In some embodiments, the method provided herein involves contacting the biological sample with an agent capable of detecting the presence or level of AR expression in the biological sample. The detection of AR expression can be based on the presence or absence of AR expression, wherein the absence of AR expression indicates that the sample is negative for AR.

AR signaling can be detected or determined using any suitable methods known in the art, including without limitation, by measuring an AR sensitive gene product, such as PSA. The level of AR sensitive gene product can be determined and compared with a reference level, wherein the detected level that is significantly lower than a reference level indicates reduced AR signaling. A reference level for AR signaling can be obtained from one or more reference samples that have been determined to have a reference level of AR signaling in a comparable subject (e.g., samples obtained from a database), which includes a collection of data, standard, or level from one or more reference samples. In some embodiments, such collection of data, standard or level are normalized.

Reduced AR signaling can also be determined based on the treatment with androgen deprivation therapy, or presence of inactivating mutations in AR. Mutation status or expression level of AR at DNA or RNA level can be measured by any methods known in the art, for example, without limitation, an amplification assay, a hybridization assay, or a sequencing assay. Mutation status or expression level of AR at protein level can be measured by any methods known in the art, for example, without limitation, immunoassays.

In some embodiments, the subject is determined to have likelihood of responsiveness to a GREM1 antagonist when the subject is detected to be absent in AR expression or signaling, or is detected to have reduced AR expression or signaling relative to a reference level.

In some embodiments, the method further comprises recommending the subject to test GREM1 expression, when the subject is detected to be absent in AR expression or signaling, or is detected to have reduced AR expression or signaling relative to a reference level.

In some embodiments, the method further comprises detecting GREM1 expression in a biological sample from the subject.

GREM1 expression can be detected using any suitable methods known in the art. In some embodiments, the method provided herein involves contacting the biological sample with an agent capable of detecting the presence or level of GREM1 expression in the biological sample. The detection of GREM1 expression can be based on the presence or absence of GREM1 expression, wherein the presence of GREM1 expression indicates that the sample is positive for GREM1.

Alternatively, the detection can be based on the level of GREM1 expression, wherein the detected level that is higher than a reference level indicates GREM1-positivity. A reference level can be obtained from one or more reference samples (e.g., samples obtained from healthy subjects, from healthy tissues or even precancerous tissues of a tumor patients). The detection of GREM1 expression can be conducted in parallel in the reference sample and the biological sample of interest. A reference level can also be obtained from a database, which includes a collection of data, standard, or level from one or more reference samples. In some embodiments, such collection of data, standard or level are normalized.

In some embodiments, when GREM1 expression is not detected in the biological sample, the method further comprising monitoring GREM1 expression in the subject after a course of time, for example, after a month, after two months, after three months, and so on.

In some embodiments, the subject is determined to have likelihood of responsiveness to a GREM1 antagonist when the subject is detected to have GREM1 expression or an elevated GREM1 expression relative to a reference level.

In another aspect, the present disclosure provides a method of detecting presence or amount of GREM1 in a sample determined to be absent in AR expression or determined to have reduced androgen receptor (AR) signaling, comprising contacting the sample with a detection reagent for detection of GREM1, and determining the presence or the amount of GREM1 in the sample.

In some embodiments, the sample is obtained from a subject having or suspected of having a cancer, as disclosed herein.

In some embodiments, the method further comprises administering a therapeutically effective amount of a GREM1 antagonist (for example any of the anti-GREM1 antibody or antigen-binding fragments thereof provided herein) to the subject determined to have likelihood of responsiveness to a GREM1 antagonist.

### PTEN/p53 Detection

In another aspect, the present disclosure provides a method of determining likelihood of responsiveness to a GREM1 antagonist in a subject having or suspected of having a disease or condition, comprising: (a) detecting deficiency of PTEN and/or p53 in a biological sample from the subject, and (b) determining the likelihood of responsiveness based on the deficiency of PTEN and/or p53 detected in step (a).

Deficiency in activity or level of PTEN and/or p53 can result in PTEN and/or p53 having no or less than normal function, or an absence of or reduced expression level of functional PTEN and/or p53 in a biological sample.

In some embodiments, the method further comprises detecting expression level of functional PTEN and/or p53 using any suitable methods known in the art, for example, without limitation, an amplification assay, a hybridization assay, a sequencing assay, or immunoassays. In some embodiments, the method provided herein involves contacting the biological sample with an agent capable of detecting the presence or level of functional PTEN and/or p53 in the biological sample. The detection of functional PTEN and/or p53 expression can be based on the presence or absence or level of functional PTEN and/or p53, wherein the absence or reduced level of functional PTEN and/or p53 indicates that the sample is deficient in in activity or level of PTEN and/or p53. In some embodiments, the method further comprises detecting mutation status of PTEN and/or p53, for example, at DNA or RNA level.

In some embodiments, the subject is determined to have likelihood of responsiveness to a GREM1 antagonist when the subject is detected to be deficient in PTEN and/or p53.

In some embodiments, the method further comprises recommending the subject to test GREM1 expression, when the subject is detected to be deficient in PTEN and/or p53.

In some embodiments, the method further comprises detecting GREM1 expression in a biological sample from the subject. Similarly, GREM1 expression can be detected and determined using any similar methods describe above.

In some embodiments, the subject is determined to have likelihood of responsiveness to a GREM1 antagonist when the subject is detected to have GREM1 expression.

In some embodiments, when GREM1 expression is not detected in the biological sample, the method further comprising monitoring GREM1 expression in the subject after a course of time, for example, after a month, after two months, after three months, and so on.

In one aspect, the present disclosure provides a method of detecting presence or amount of GREM1 in a sample determined to be deficient in PTEN and/or p53, comprising contacting the sample with a detection reagent for detection of GREM1, and determining the presence or the amount of GREM1 in the sample.

In some embodiments, the sample is obtained from a subject having or suspected of having a GREM1 related disease or condition, as disclosed herein.

In some embodiments, the method further comprises administering a therapeutically effective amount of a GREM1 antagonist (for example any of the anti-GREM1 antibody or antigen-binding fragments thereof provided herein) to the subject determined to have likelihood of responsiveness to a GREM1 antagonist.

### Biological Sample

The presence and/or expression level and/or mutation status of a biomarker (e.g. AR, PTEN, p53, and/or GREM1) can be determined using a suitable biological sample obtained from the subject.

In some embodiments, the biological sample contains or is suspected to contain a cancer cell. In some embodiments, the biological sample is obtained from a cancer microenvironment. In some embodiments, the biological sample can be obtained or derived from the subject, for example, as formalin fixed paraffin embedded (FFPE) tissue, fresh biopsy, blood (suspected of containing circulating tumor cells), or other body fluid. In some embodiments, the cancer cell, stromal cell and/or extracellular matrix may be isolated from the biological sample. In certain embodiments, the biological sample may be further processed to, for example, isolate the analyte such as the nucleic acids or proteins.

In certain embodiments, the biological sample comprises a cancer cell, stromal cell, stroma or a fibrotic cell.

### Detection and/or Determination

As used herein, the terms “determining”, “measuring” and “detecting” can be used interchangeably and refer to both quantitative and semi-quantitative determinations.

The biomarkers AR, PTEN, p53 and/or GREM1 provided herein are intended to encompass different forms including mRNA, protein and also DNA (e.g. genomic DNA). Therefore, the level and/or activity of these biomarkers can be measured with RNA (e.g. mRNA), protein or DNA (e.g. genomic DNA) of the respective biomarker. Similarly, mutation status of the biomarkers can also be measured with DNA (e.g. genomic DNA), RNA (e.g. mRNA), or protein (for example by measuring for an altered protein product encoded by the mutated gene).

Expression level of a biomarker at DNA or RNA level can be measured by any methods known in the art, for example, without limitation, an amplification assay, a hybridization assay, or a sequencing assay, using techniques including, without limitation, RNA sequencing (RNA-seq) and RNAscope (Wang, Z., Gerstein, M., & Snyder, M. (2009). RNA-seq: a revolutionary tool for transcriptomics. Nature Reviews Genetics, 10(1), 57-63; Wang et al., RNAscope: a novel in situ RNA analysis platform for formalin-fixed, paraffin-embedded tissues, J Mol Diagn. 2012 January; 14(1): 22-9.). Expression level of a biomarker at protein level can be measured by any methods known in the art, for example, without limitation, immunoassays (such as Western blotting, enzyme-linked immunosorbent assay (ELISA), enzyme immunoassay (EIA), radioimmunoassay (RIA), sandwich assays, competitive assays, immunofluorescent staining and imaging, immunohistochemistry (IHC), and fluorescent activating cell sorting (FACS)).

Mutation status of a biomarker at DNA or RNA level can be measured by any methods known in the art, for example, without limitation, an amplification assay, a hybridization assay, or a sequencing assay. Mutation status at protein level can be measured by any methods known in the art, for example, without limitation, immunoassays.

Activity level of a biomarker can be measured by a suitable functional assay known in the art.

These methods are well-known in the art, and are described in detail below as exemplary illustration.

i. Amplification Assay

A nucleic acid amplification assay involves copying a target nucleic acid (e.g. DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (See Abravaya, K., et al., Nucleic Acids Research, 23:675-682, (1995), branched DNA signal amplification (see, Urdea, M. S., et al., AIDS, 7 (suppl 2):S11-S14, (1993), amplifiable RNA reporters, Q-beta replication (see Lizardi et al., Biotechnology (1988) 6: 1197), transcription-based amplification (see, Kwoh et al., Proc. Natl. Acad. Sci. USA (1989) 86: 1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

### Hybridization Assay

Nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

In certain embodiments, the probes for hybridization assay are detectably labeled. In certain embodiments, the nucleic acid-based probes for hybridization assay are unlabeled. Such unlabeled probes can be immobilized on a solid support such as a microarray, and can hybridize to the target nucleic acid molecules which are detectably labeled.

In some embodiments, hybridization assays can be performed on microarrays.

### Sequencing Methods

Sequencing methods allow determination of the nucleic acid sequence of the target nucleic acid, and can also permit enumeration of the sequenced target nucleic acid, thereby measures the level of the target nucleic acid. Examples of sequence methods include, without limitation, RNA sequencing, pyrosequencing, and high throughput sequencing.

High throughput sequencing involves sequencing-by-synthesis, sequencing-by-ligation, and ultra-deep sequencing (such as described in Marguiles et al., Nature 437 (7057): 376-80 (2005)). Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina® sequencing platform.

In certain embodiments, the detection of mutation and/or wild-type status and the measurement of level of biomarkers of interest described herein is by whole transcriptome sequencing, or RNA sequencing (e.g. RNA-Seq). Briefly, the RNA-seq comprises reverse transcribing a target mRNA into a cDNA, fragmenting and sequencing the cDNA and analyzing the sequence data for mRNA quantification; the RNAscope comprises in situ hybridizing a target mRNA with one or more oligonucleotides conjugated with a fluorescent probe and detecting the level of mRNA by measuring the fluorescence intensity.

### Immunoassays

Immunoassays typically involves using antibodies that specifically bind to the biomarker polypeptide or protein (e.g. the ATM, ATR, MDM2, and/or p53 protein as provided herein) to detect or measure the presence or level of the target polypeptide or protein. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), sandwich assays, competitive assays, immunofluorescent staining and imaging, immunohistochemistry (IHC), and fluorescent activating cell sorting (FACS). For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7^{th} ed. 1991).

In certain embodiments, the methods of the present disclosure include measuring expression level or gene copies of AR, PTEN, p53 and/or GREM1. The activity of p53 can be measured by detecting the phosphorylation of the amino acid residue at position 15 of p53, or by detecting the change in expression level of the downstream target genes of p53. Due to a protein's ability to exert multiple biological activities, several acceptable bioassays may exist for a particular protein. Exemplary functional assays for measuring the activity of AR, PTEN, p53 and/or GREM1 can be found in Lee J-H et al, J Biol Chem, 288: 12840-12851 (2013), Loughery J, et al, Nucleic Acids Research, 42:7666-7680 (2014), Thompson T, et al, Journal Biological Chemistry, 279:53015-53022 (2004), Wienken, M. et al., J. Mol. Cell Biol. 2017; 9(1): 74-80.

In certain embodiments, a decrease (e.g. at least 5%, 10%, 15%, 20%, 25%, 30%, 35%, 40%, 45%, 50%, 55%, 60%, 65%, 70%, 75%, 80%, 85%, 90%, or 95% decrease) in expression level of ATR, PTEN and/or p53 gene product relative to a reference level of AR, PTEN and/or p53 gene product respectively, indicates deficiency in activity or level of AR, PTEN and/or p53 in the biological sample.

In certain embodiments, the expression level of the AR, PTEN and/or p53 can be normalized to an internal control value or to a standard curve. For example, the level of each of the AR, PTEN and/or p53 described herein can be normalized to a standard level for a standard marker. The standard level of the standard marker can be predetermined, determined concurrently, or determined after a sample is obtained from the subject. The standard marker can be run in the same assay or can be a known standard marker from a previous assay. In the cases when the level of the PTEN and/or p53 is determined by sequencing assay (such as RNA sequencing), the level of the biomarkers can be normalized to the total reads of the sequencing.

In certain embodiments, the method further comprises isolating the nucleic acid from the sample, if RNA or DNA level of the AR, PTEN and/or p53 is to be measured. Various methods of extraction are suitable for isolating the DNA or RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning: A Laboratory Manual 3^{rd} ed. (2001).

Commercially available kits can also be used to isolate RNA, including for example, the NucliSens extraction kit (Biomerieux, Marcy l'Etoile, France), QIAamp™ mini blood kit, Agencourt Genfind™, Rneasy® mini columns (Qiagen), PureLink® RNA mini kit (Thermo Fisher Scientific), and Eppendorf Phase Lock Gels™. A skilled person can readily extract or isolate RNA or DNA following the manufacturer's protocol.

### Kits

In another aspect, the present disclosure further provides a kit for use in the methods described herein.

In one embodiment, the kit comprises: a first reagent, or a first set of reagents, for detecting presence or absence of one or more inactivating mutation in PTEN/p53; or one or more reagents for measuring expression level of PTEN/p53. In one embodiment, wherein the kit further comprises a second reagent for detecting presence or absence or expression level of GREM1.

In one embodiment, the kit comprises: a first reagent for measuring expression level of or presence or absence of inactivating mutation of AR. In one embodiment, the kit further comprises a second reagent for detecting presence or absence or expression level of GREM1.

In certain embodiments, the first reagent comprises one or more primers, one or more probes, and/or one or more antibodies, directed to PTEN, or p53, or AR. In certain embodiments, the second reagent comprises one or more primers, one or more probes, and/or one or more antibodies, directed to GREM1. The primers, the probes, and/or the antibodies may or may not be detectably labeled.

In certain embodiments, the kits may further comprise other reagents to perform the methods described herein. In such applications the kits may include any or all of the following: suitable buffers, reagents for isolating nucleic acid, reagents for amplifying the nucleic acid (e.g. polymerase, dNTP mix), reagents for hybridizing the nucleic acid, reagents for sequencing the nucleic acid, reagents for quantifying the nucleic acid (e.g. intercalating agents, detection probes), reagents for isolating the protein, and reagents for detecting the protein (e.g. secondary antibody). Typically, the reagents useful in any of the methods provided herein are contained in a carrier or compartmentalized container. The carrier can be a container or support, in the form of, e.g., bag, box, tube, rack, and is optionally compartmentalized.

In certain embodiments, the present disclosure provides use of the first reagent provided herein, optionally with the second reagent, in the manufacture of a diagnostic reagent for use in the diagnostic methods provided herein.

In some embodiments, the present disclosure also provides use of the GREM1 antagonist (e.g. the antibody or antigen-binding fragment thereof provided herein) in the manufacture of a medicament for treating or diagnosing a GREM1-expressing cancer in a subject, wherein the GREM1-related disease or condition is determined to be deficient in PTEN and/or p53.

In some embodiments, the present disclosure also provides use of the GREM1 antagonist (e.g. the antibody or antigen-binding fragment thereof provided herein) in the manufacture of a medicament for treating or diagnosing a GREM1-related disease or condition in a subject, wherein the GREM1-related disease or condition is determined to be deficient in PTEN and/or p53.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1: Upregulation of Gremlin1 in Prostate Cancers (PCas) Strongly Correlates with the Development of Castration Resistance and a Poor Disease Outcome

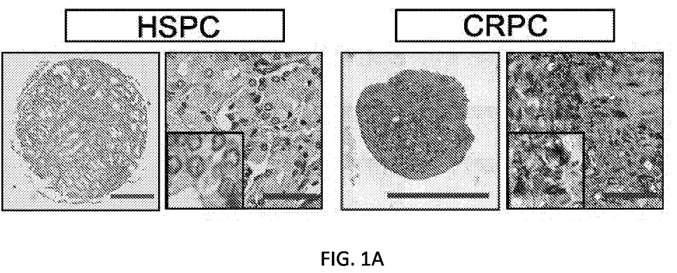

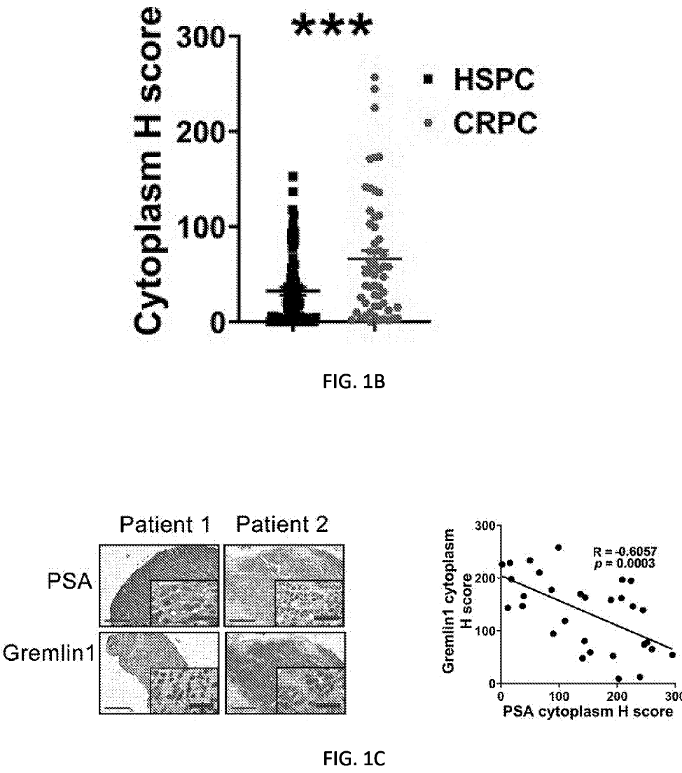

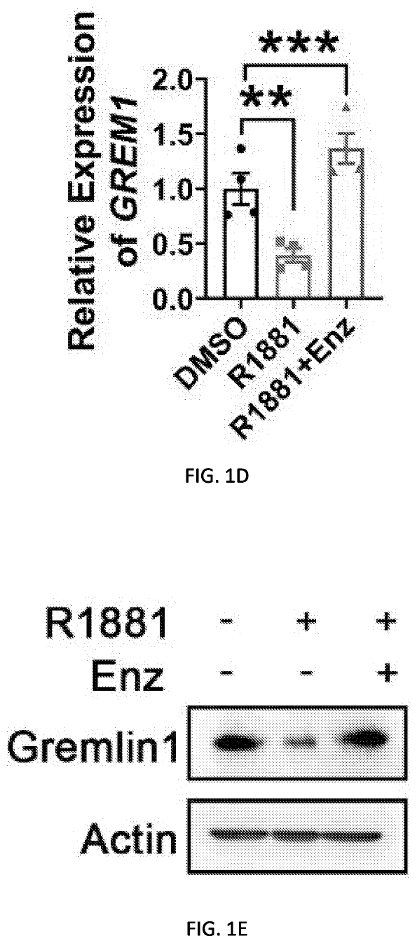

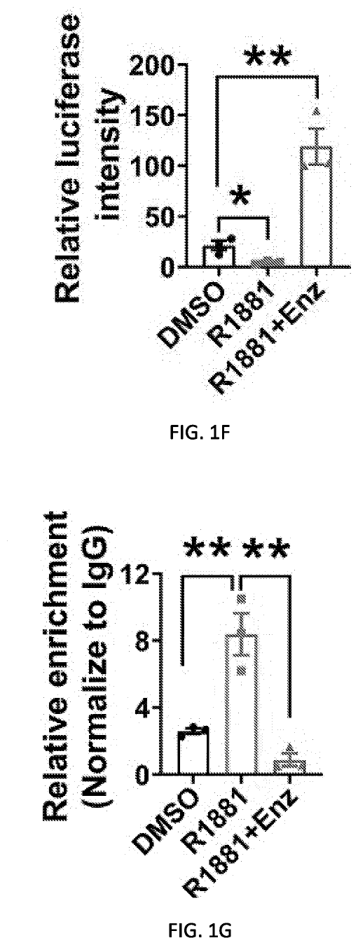

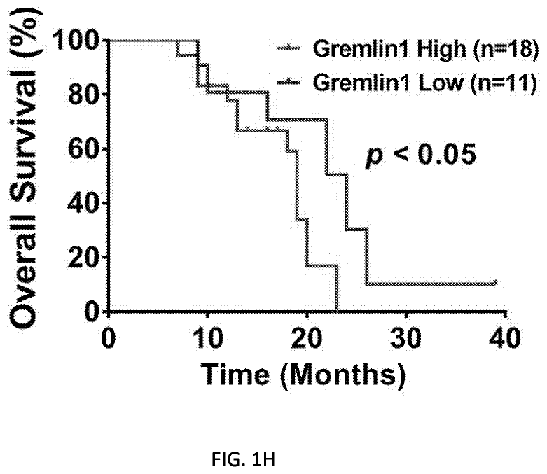

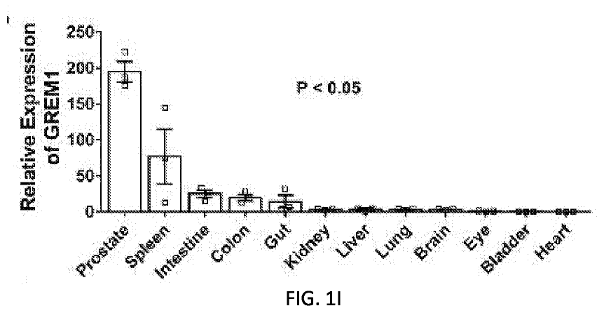

Secreted protein is a group of important potential therapeutic target for anti-cancer drug development. To screen specifically upregulated secreted proteins in in castration-resistant prostate cancer (CRPC), we performed data mining in published RNA-sequencing datasets. The expression of Gremlin1 was ranked at the top differentially expressed genes encoding secreted proteins in hormone refractory PCa (Best, C. J., et al. Molecular alterations in primary prostate cancer after androgen ablation therapy. Clin Cancer Res 11, 6823-6834 (2005)). Additional analysis on other PCa datasets suggested that Gremlin1 expression levels increased significantly in advanced metastatic CRPC than primary PCa, or in hormone refractory PCa compared to hormone naïve PCa (based on the sequencing data from Yu, Y. P., et al. Gene expression alterations in prostate cancer predicting tumor aggression and preceding development of malignancy. J Clin Oncol 22, 2790-2799 (2004); Best, C. J., et al. Molecular alterations in primary prostate cancer after androgen ablation therapy. Clin Cancer Res 11, 6823-6834 (2005)). Importantly, amplification of GREM1 was associated with shortened disease/progression-free survival based on data analysis on Prostate Adenocarcinoma (TCGA, Firehose Legacy). We then performed Gremlin1 immunohistochemical (IHC) staining on a large cohort of 139 human PCa patients at Ren Ji Hospital, School of Medicine, Shanghai Jiao Tong University. Among the 139 patient specimens, 60 samples were obtained from castration resistance prostate tumor patients. Quantitative study of the IHC result showed a significantly enhanced staining intensity of Gremlin1 in CRPC samples than hormone sensitive PCa (HSPC) (FIG. 1A, 1B). Patients with higher Gremlin1 expression in PCa had a notably shorter overall survival (FIG. 1H). Based on the sequencing data of the SU2C 2019 PCa dataset (A Robinson et al., Cell 161(5), 1215-1228 (2015)), it also revealed that patients with elevated transcription of GREM1 exhibited a shorter overall survival. Collectively, Gremlin1 was upregulated in CRPCs and strongly correlates with poor disease outcome.

### Example 2: Transcription of GREM1 is Repressed by AR and Increases after ADT

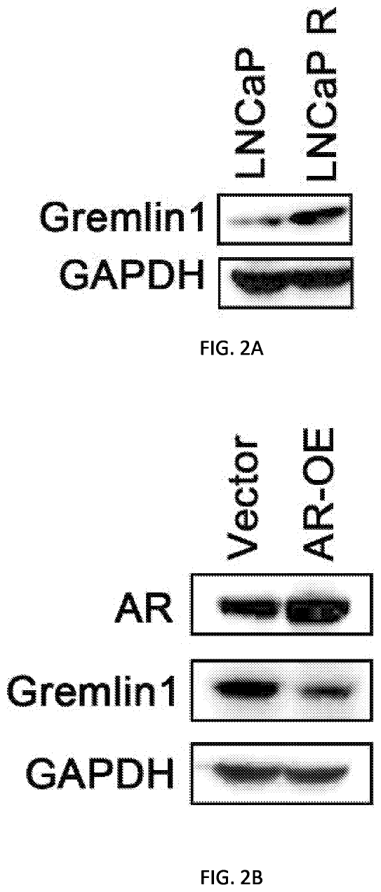

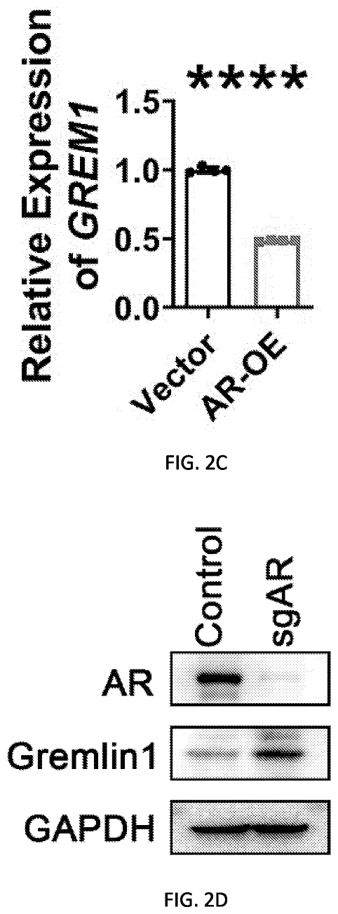

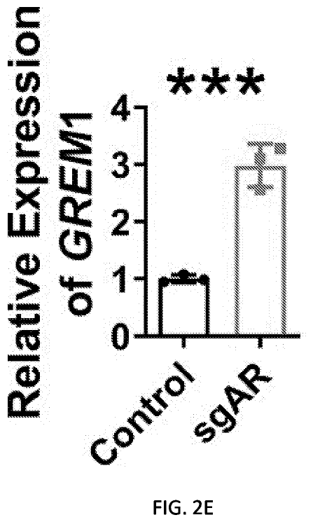

AR plays a central role in PCa. To assess the relationship between Gremlin1 and AR signaling, we performed further IHC staining for Gremlin1 and PSA, a classic downstream target of AR, on sections of CRPC specimens. Statistical analysis showed that Gremlin1 expression was evidently upregulated in CRPCs with a low staining intensity of PSA (FIG. 1C). In addition, we detected a significant increase of GREM1 in a castration resistant LNCaP cell line (termed as LNCaP R), which was derived from xenograft LNCaP tumors implanted in castrated mice, compared to a control LNCaP cell line generated from xenografts in intact mice (FIG. 2A). We then asked whether the expression of GREM1 was regulated by AR signaling. We utilized an AR expressing lentivirus or the CRISPR/Cas9 method to achieve AR upregulation (FIG. 2B) or knockout in the LNCaP PCa cell line (FIG. 2D). Immunoblotting and q-PCR experiments together showed that AR repressed the transcription of GREM1 (FIGS. 2B-2E). This conclusion was supported by AR agonist R1881 and antagonist Enzalutamide treatment experiments (FIG. 1D, 1E).

Additionally, we carried out a luciferase reporter assay. The GREM1 promoter driven luciferase activity was greatly inhibited by the treatment of R1881, while enhanced by the addition of enzalutamide (FIG. 1F). ChIP experimental results further suggested a binding of AR to the promotor region of GREM1 (FIG. 1G). These results together demonstrated that GREM1 was elevated in CRPC and was negatively regulated by AR.

### Example 3: GREM1 Promotes PCa Cell Proliferation and Tumor Growth Upon Androgen Deprivation

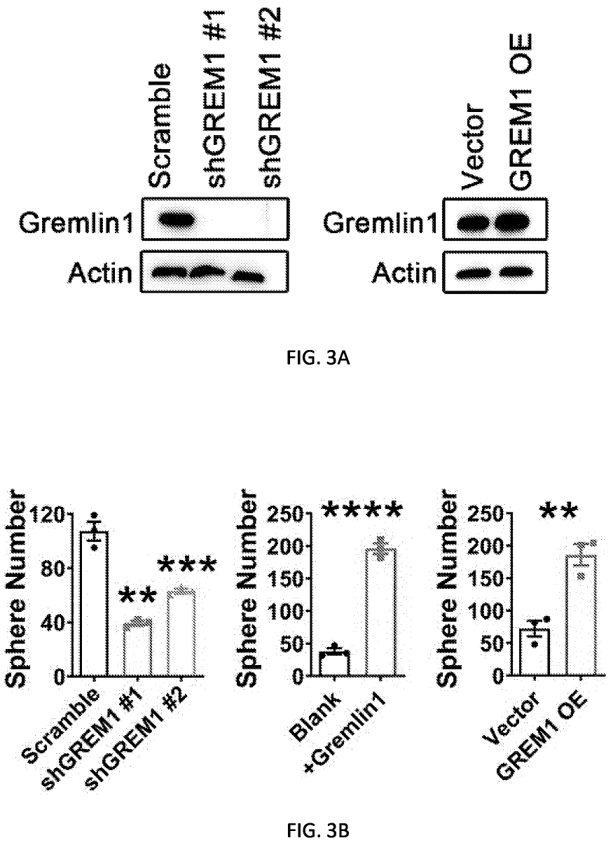

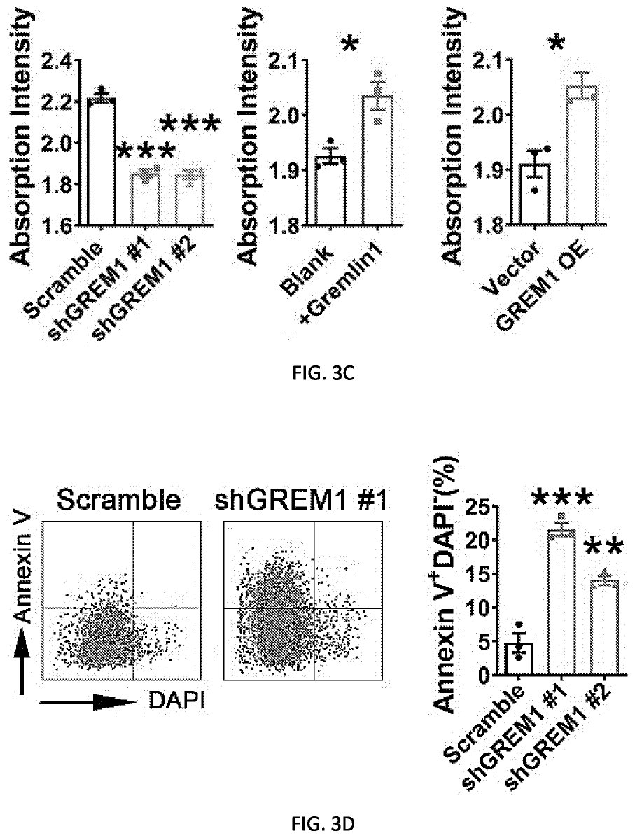

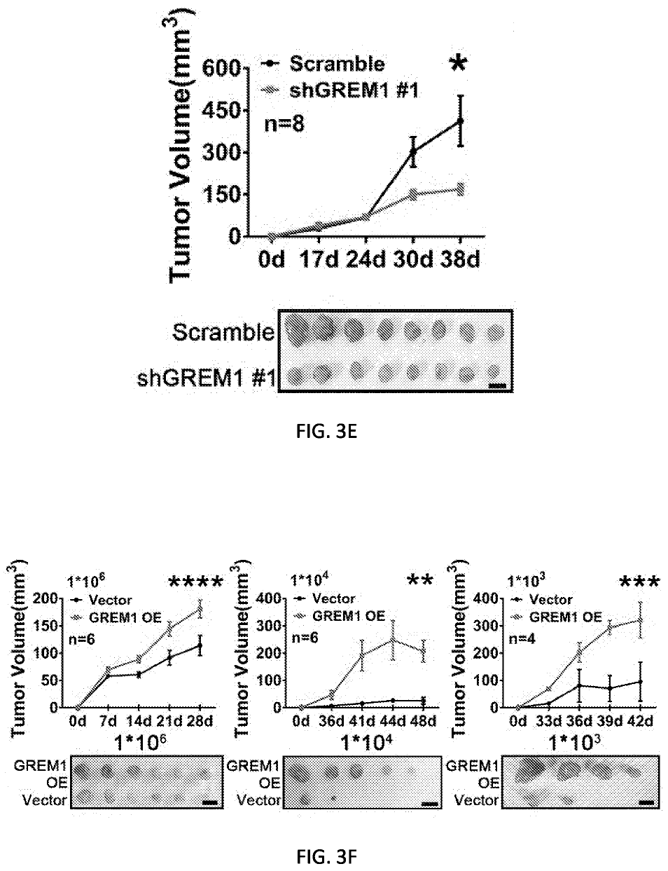

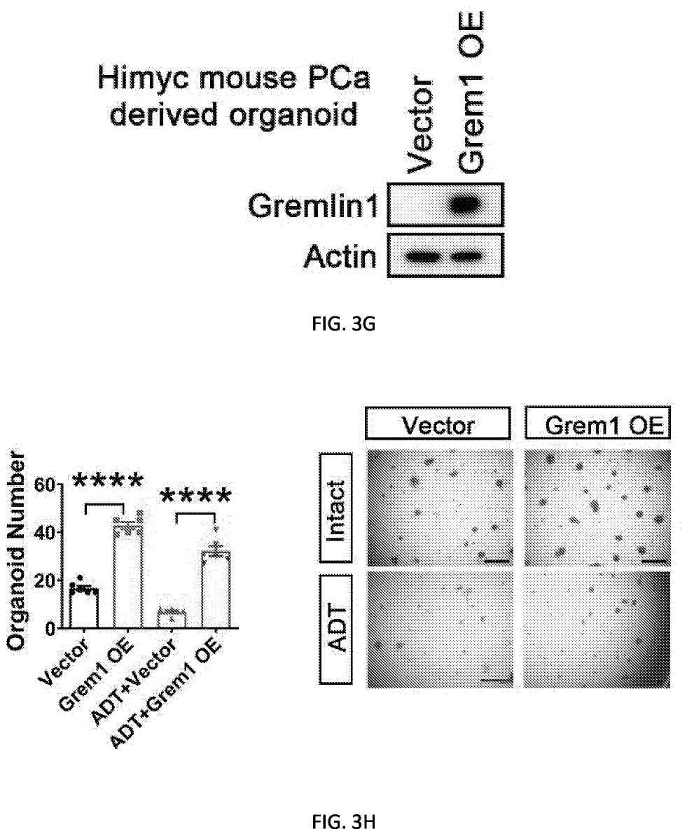

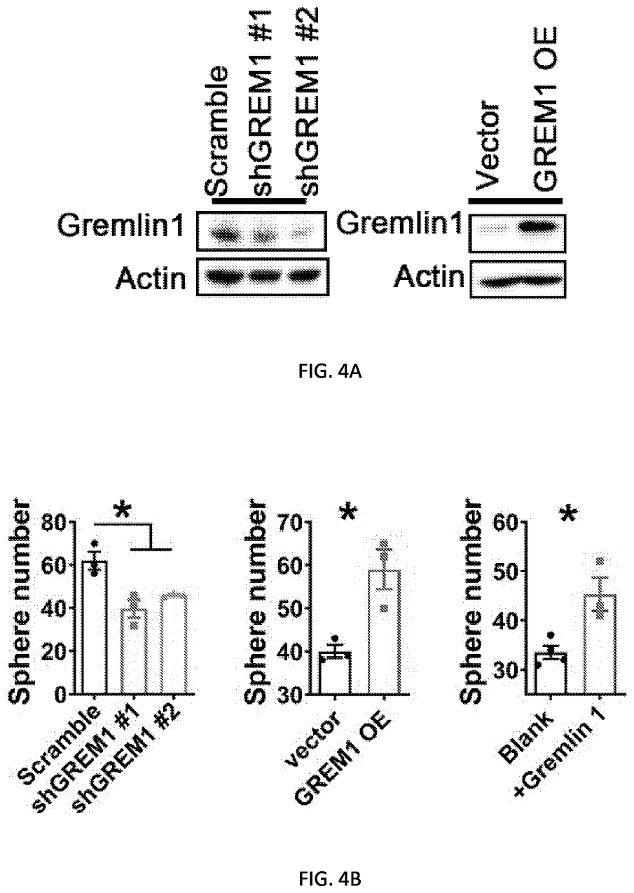

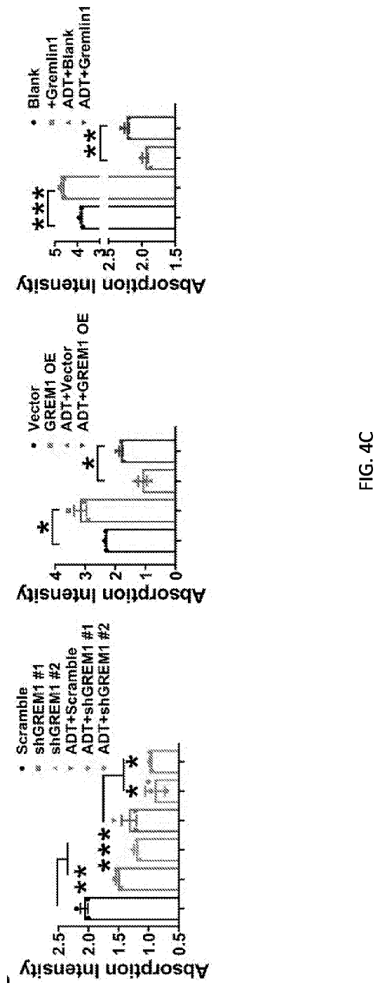

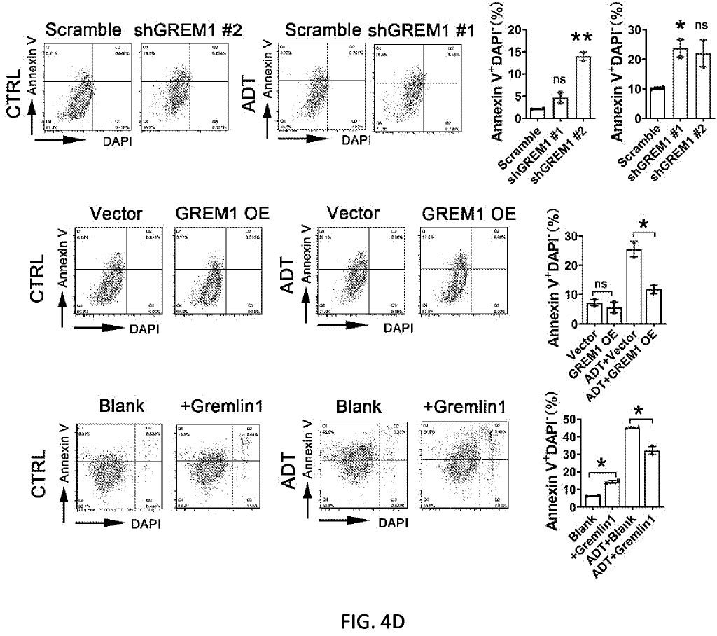

Metastatic prostate cancer is a devastating disease and most cancers progress upon serial treatments with either androgen receptor antagonist or chemotherapy. One of the key cell types resistant to these treatments are cells with stem cells like property which has the capability of forming tumor spheres in suspension culture. To explore the role of GREM1 in the progression of CRPC, we utilized AR independent CRPC cell line PC3, as well as the AR dependent PCa cell line LNCaP and LAPC4. We generated cell sublines with loss or gain of GREM1 expression (FIGS. 3A, 4A and 5A). GREM1 knockdown in PC3 suppressed sphere forming capacity, cell growth and survival, whereas GREM1 overexpression or addition of 100 ng/ml GREM1 protein in culture medium resulted in a significant elevation in sphere formation and cell proliferation than corresponding control sublines (FIGS. 3B, 3C, 3D and 4B). Moreover, we found that the knockdown of GREM1 markedly suppressed the PC3 tumor growth in vivo, while overexpression of GREM1 enhanced the tumor growth and tumor forming incidence in a limiting dilution assay (FIG. 3E, 3F).

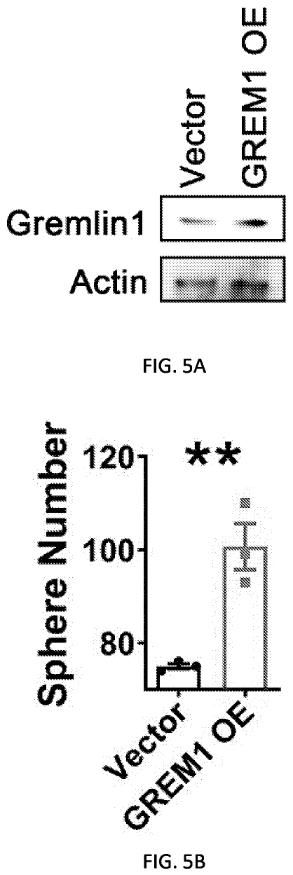

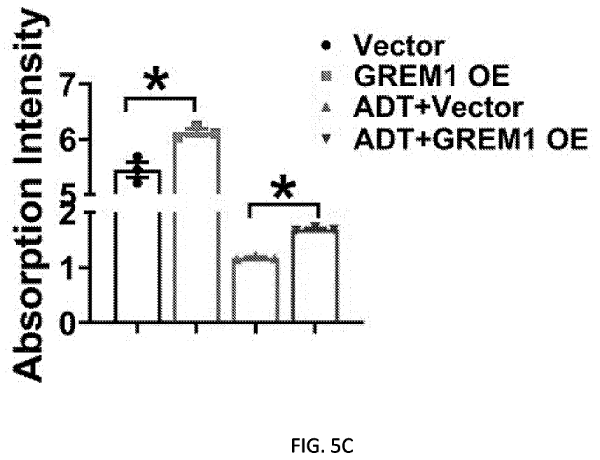

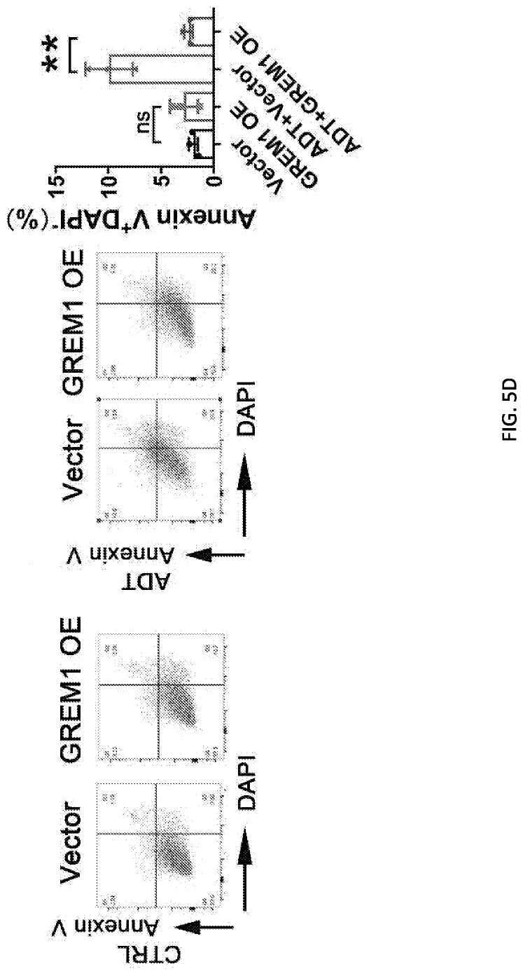

Furthermore, exogenous expression of GREM1 in PCa organoids generated from the Hi-Myc mouse, a genetically engineered mouse model (GEMM) for PCa, promotes organoid growth in the androgen deprived condition (FIGS. 3G and 3H). GREM1 knockdown greatly potentiated the inhibitory effect of enzalutamide to AR-dependent LNCaP and LAPC4 cells, while GREM1 overexpression or addition of GREM1 protein led to a compromised response to the enzalutamide treatment in LNCaP and LAPC4 cells (FIGS. 4A-4D and 5A-5D). Those data together suggested a tumor promoting role of GREM1 in PCa and the development of castration resistance.

### Example 4: The Oncogenic Effect of GREM1 in PCa is Dependent on the Activation of the FGFR1/MEK/ERK Signaling Pathway

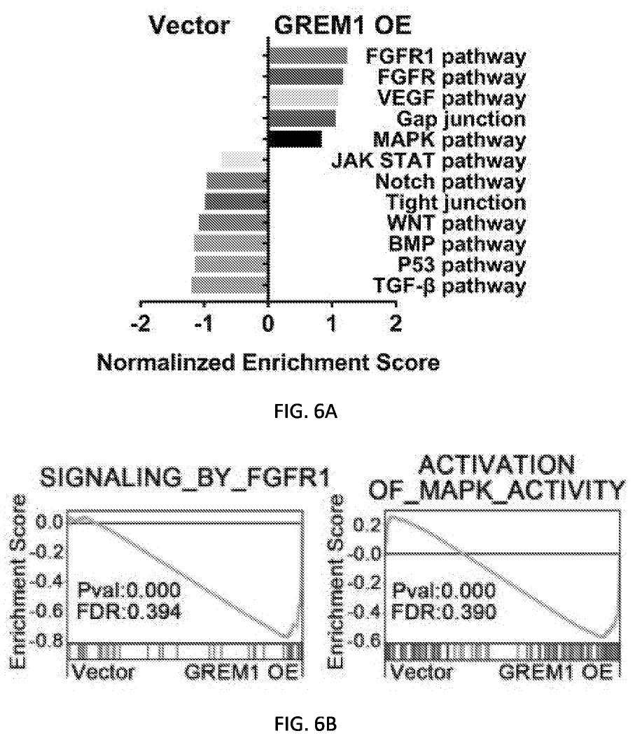

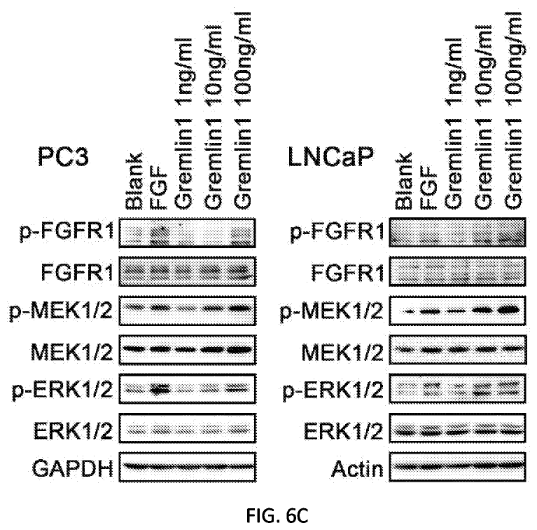

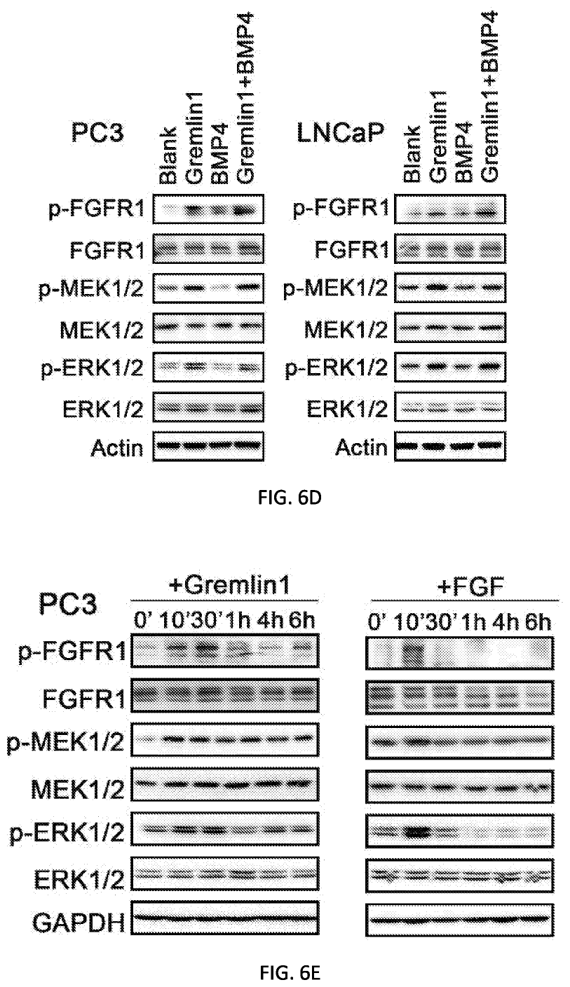

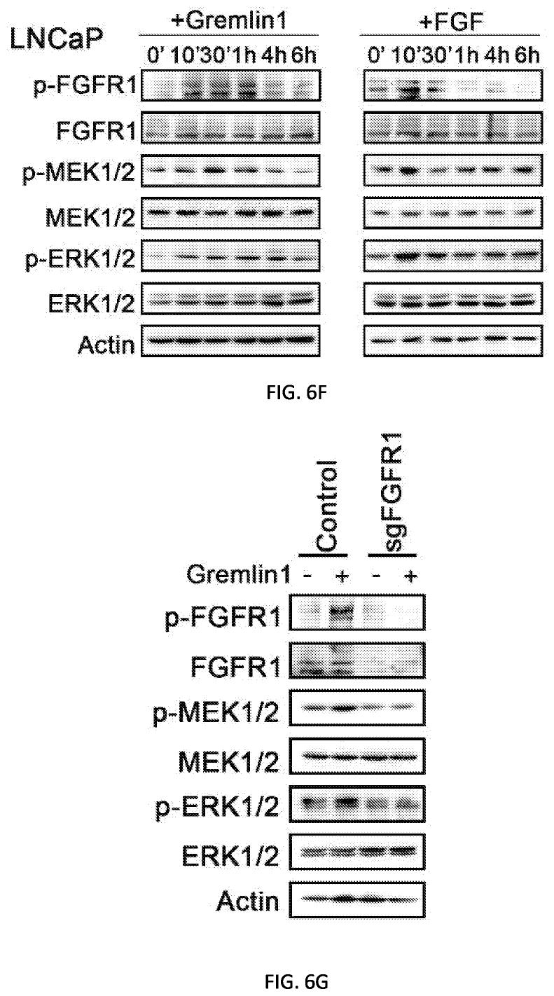

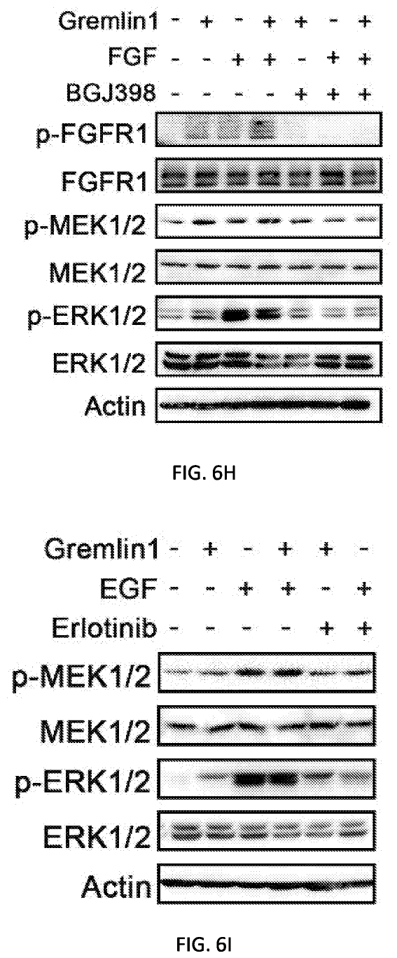

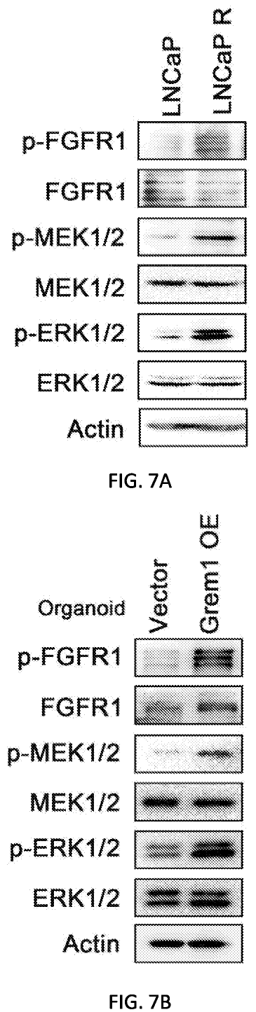

To address the mechanism underlying the oncogenic effect of GREM1, we performed RNA-sequencing to compare the transcriptional difference between GREM1-overexpressing LNCaP sublines and their control cells. We listed the most significantly differential expressing gene sets in FIG. 6A. FGFR and MAPK signaling were the top hits in the upregulated signaling pathways. Further gene set enrichment analysis (GSEA) showed an enrichment in signaling by FGFR1 and activation of MAPK activity in LNCaP cells transfected with the GREM1 expressing lentivirus (FIG. 6B). Moreover, in addition to an upregulation of GREM1 (FIG. 2A), we found that MAPK and FGFR1 were both activated in castration resistant LNCaP cells compared to hormone naïve LNCaP cells (FIG. 7A). This is particularly relevant in light of recent findings that the FGF signal activation is required for the AR-independent growth of CRPC.

In order to test whether Gremlin1 promoted the FGFR-MAPK signaling, we first performed analysis of the expression levels of the four FGFRs in CRPC patients. Based on the sequencing data of SU2C CRPC cohort (Robinson et al., Cell 161(5), 1215-1228 (2015)), FGFR1 was the most abundantly expressed FGFR in CRPC. Therefore, we mainly examined the FGFR1 activation following GREM1 treatment. We treated LNCaP and PC3 with GREM1 in different concentrations (1 ng/ml, 10 ng/ml, 100 ng/ml) and examined phosphorylation levels of FGFR1, MEK and ERK. We used the known FGFR1 ligand FGF1 as a positive control. As shown in FIG. 6C, GREM1 treatment led to an increase of p-FGFR1, p-MEK1/2 and p-ERK1/2 in a dose dependent manner. We further uncovered that activation of the FGFR1-MAPK axis by GREM1 was independent on BMP, because addition of BMP4 did not alter the phosphorylation levels of the FGFR1, MEK1/2 and ERK1/2 following GREM1 stimulation (FIG. 6D). Interestingly, we found that GREM1 induced a more prolonged activation MAPK signaling than FGF1. The activation of MAPK in response to GREM1 treatment was maintained at a high level after GREM1 was added for up to 1 hour, while the MAPK signaling was most highly activated in 10 minutes upon FGF1 stimulation and quickly diminished afterwards (FIG. 6E, 6F). In addition, exogenous expression of GREM1 led to an activation of MAPK/FGFR1 signaling axis in PCa organoids derived from the Hi-myc murine PCa model. (FIG. 7B).

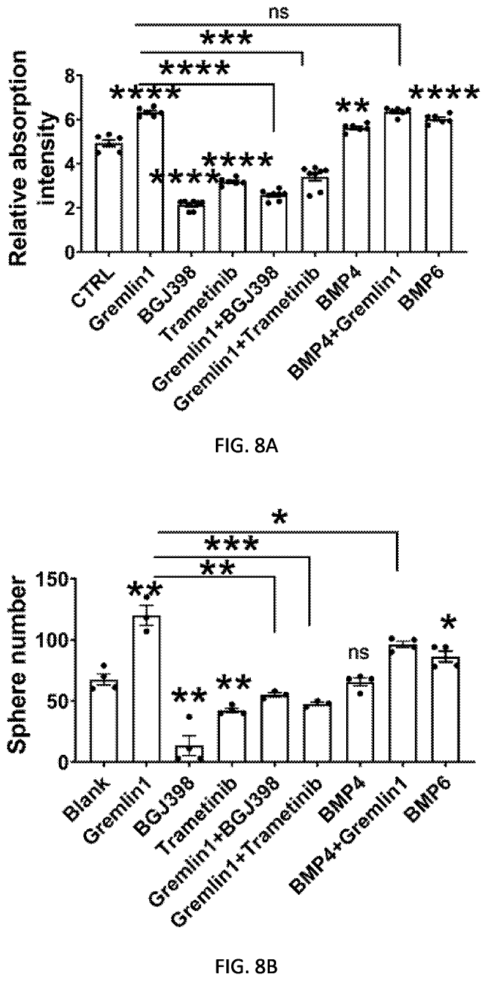

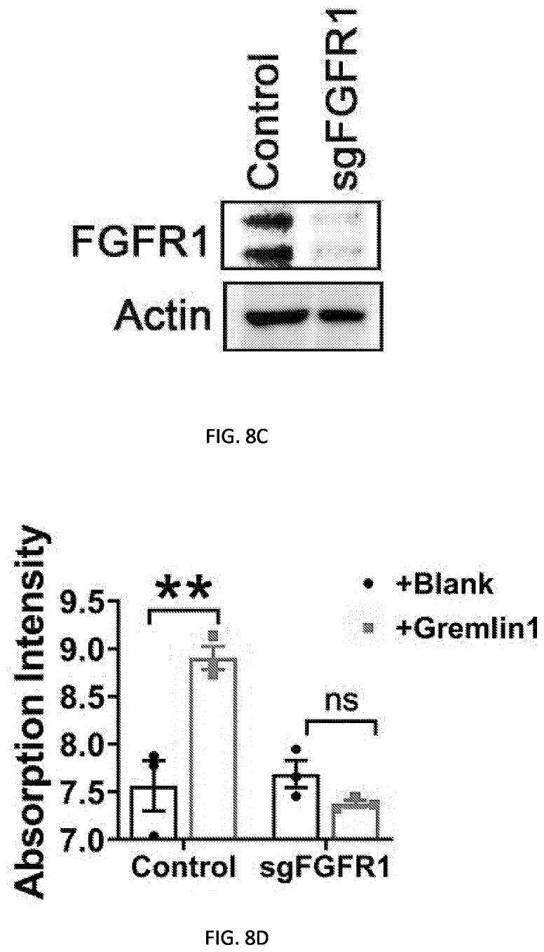

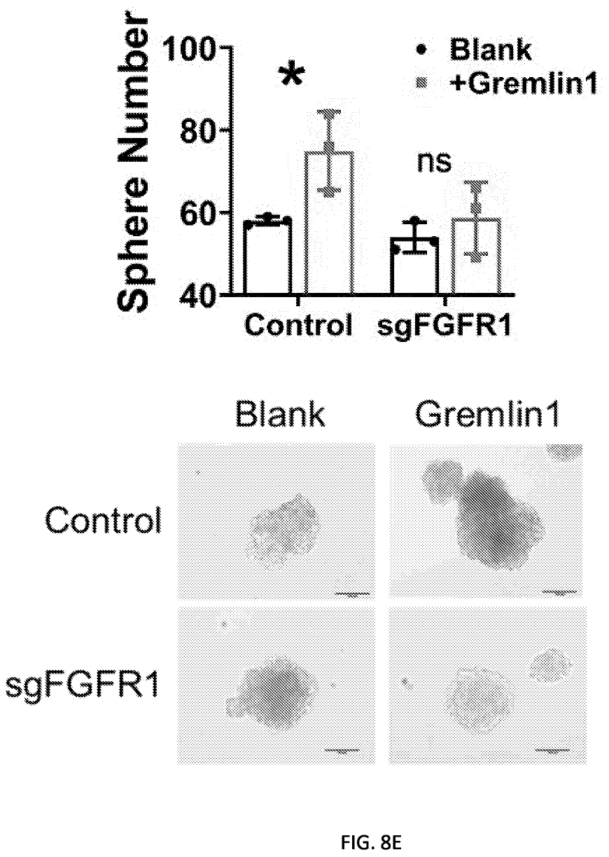

MAPK signaling can be activated through many membrane receptors besides FGFR. To test whether the activation of MAPK pathway by GREM1 was via FGFR, we constructed a FGFR1 knockout LNCaP subline by the CRISPR/Cas9 method. As shown in FIG. 6G, phosphorylation of ERK1/2 and MEK1/2 by treatment of GREM1 can be abrogated by FGFR1 knockout. Additionally, GREM1 mediated promoting effect on PCa cell growth and sphere formation can be abolished by knockout of FGFR1 (FIG. 8A-8E). To further test whether the tumor promoting effect of GREM1 was through FGFR, we utilized small molecule inhibitors of FGFR or EGFR. Activation of MAPK/FGFR1 signaling axis by GREM1 can be attenuated by the FGFR1 inhibitor BGJ398 but not the EGFR inhibitor Erlotinib (FIG. 6H, 6I). Furthermore, as shown in FIG. 8A, B, the PCa proliferation and sphere-formation promoting roles of GREM1 were significantly compromised by treatment with the FGFR inhibit BGJ398 or MEK inhibitor Trametinib, but not affected by addition of BMP4. These results indicated that the oncogenic effect of Gremin1 was attributable to the activation of the FGFR1/MEK/ERK signaling pathway.

### Example 5: GREM1 is a Novel FGFR1 Ligand in PCa

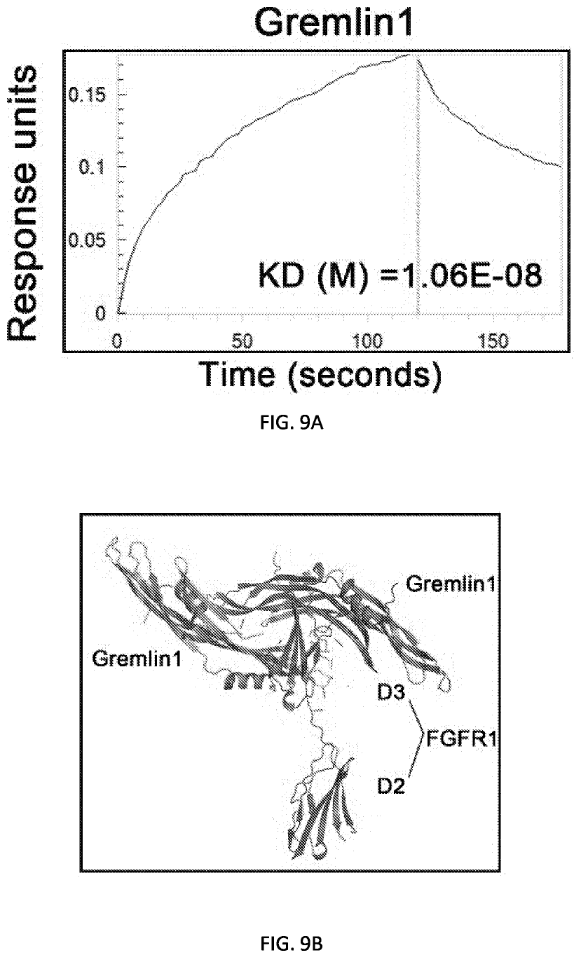

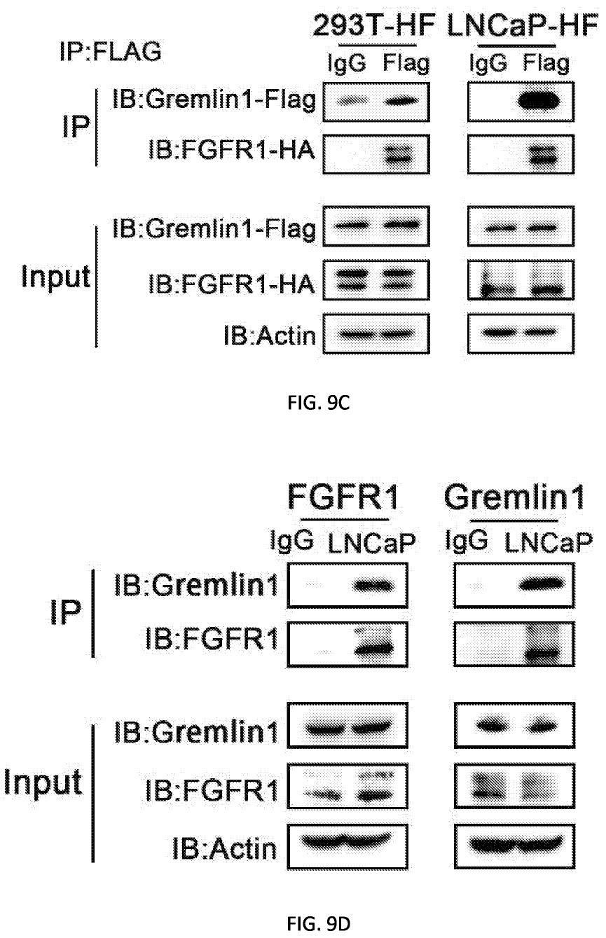

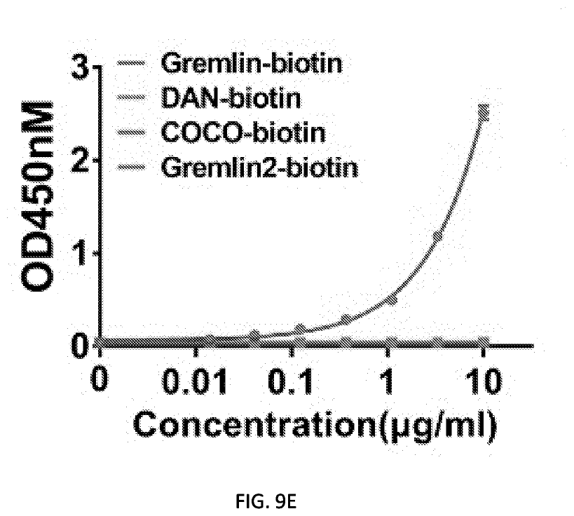

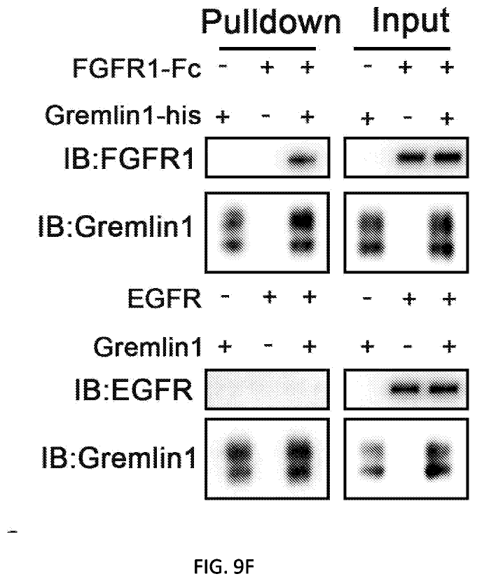

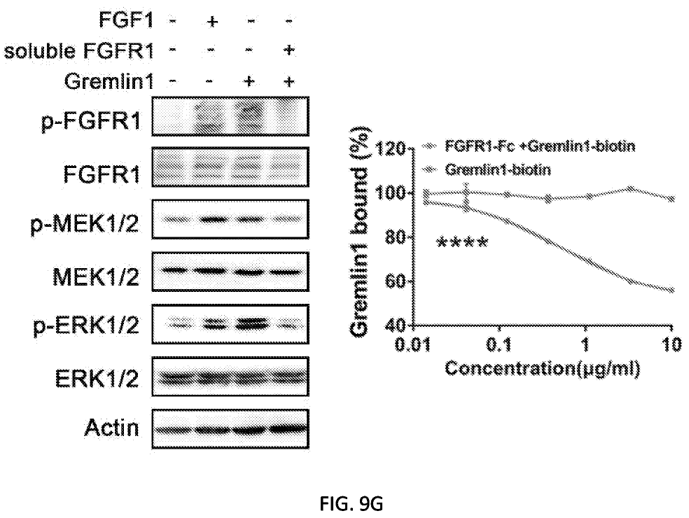

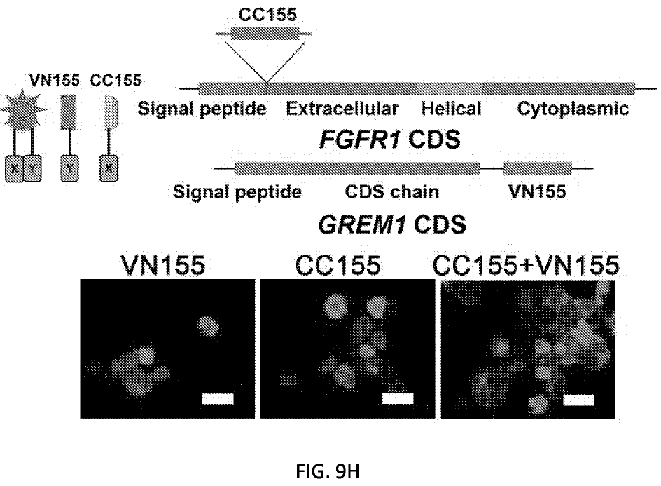

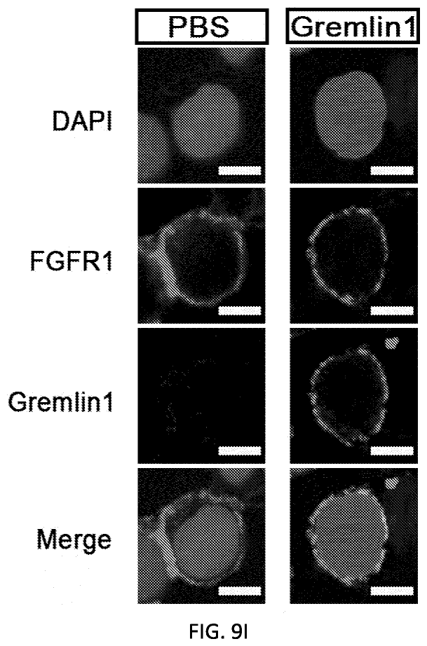

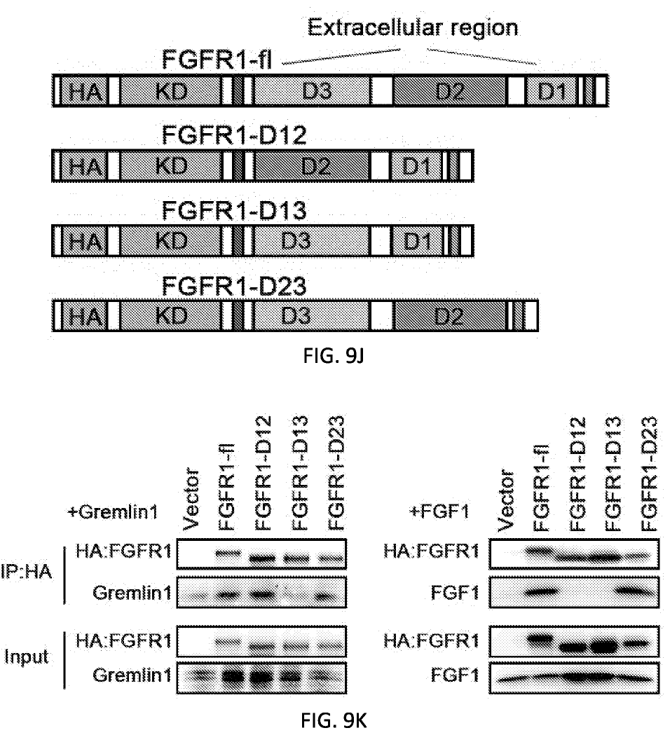

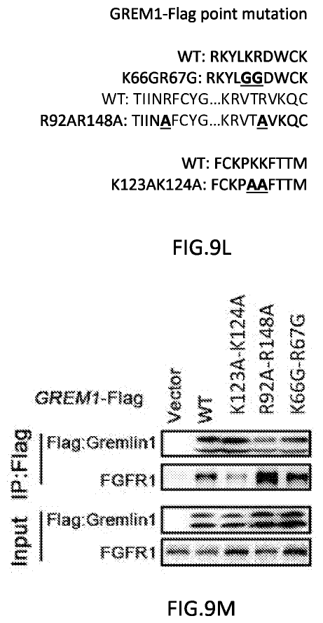

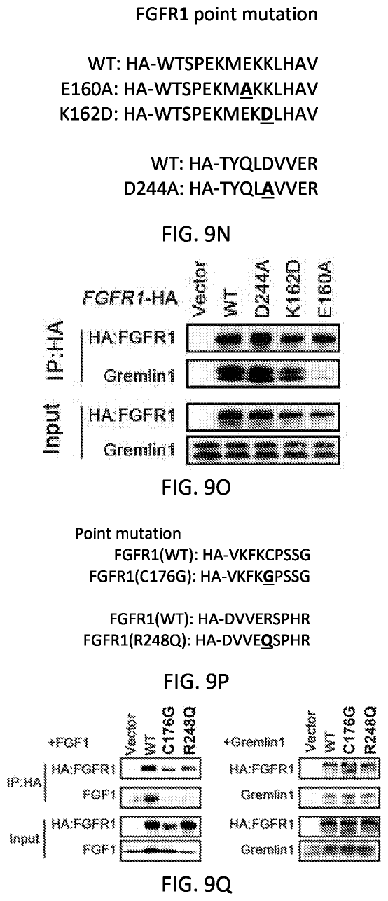

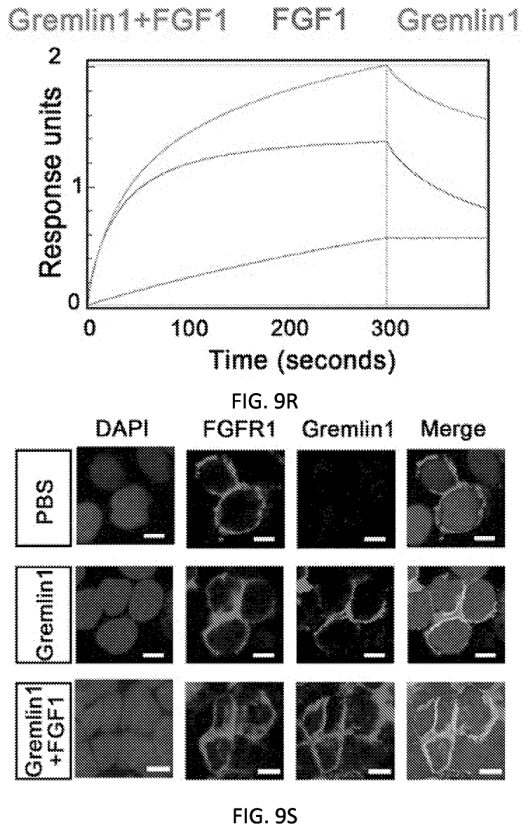

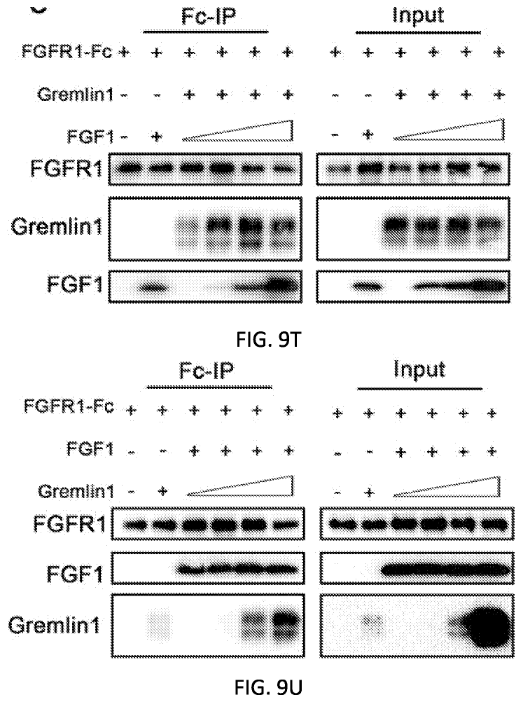

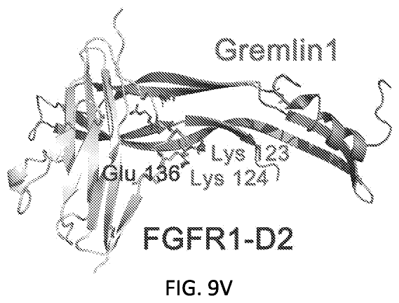

We next asked the mechanism leading to the activation of the FGFR1/MEK/ERK signaling pathway by GREM1. We performed surface plasmon resonance analysis (Fortebio) to first assess whether GREM1 can bind to FGFR1. As shown in FIG. 9A, FGFR1 bound to GREM1 immobilized on a ForteBio sensor chip with a high affinity (KD=1.06E-08 M). Computer simulation imitated that GREM1 binds to the extracellular domain of FGFR1 as a dimer (FIG. 9B). The binding between FGFR1 and GREM1 was further substantiated by co-immunoprecipitation assay using exogenously expressed Flag-tagged GREM1 and HA-tagged FGFR1 in both 293T cells and LNCaP cells (FIG. 9C), or endogenous proteins in LNCaP cells (FIG. 9D). Direct association between FGFR1 and GREM1 was supported by pull-down experiments as shown in FIG. 9F. FIG. 9E further shows that Gremlin 1 but not Gremlin 2 or other members of DAN protein family binds to FGFR1 as measured by Enzyme-linked immunosorbent assay (ELISA). In addition, the activation of GREM1 on the FGFR1/MEK/ERK signaling pathway could be attenuated by adding excessive amount of soluble FGFR1 (FIG. 9G).

We conducted a Bimolecular Fluorescence Complementation (BiFC) assay to test the interaction of GREM1 and FGFR1. GREM1 and FGFR1 cDNA fused with fragments of coding sequence of yellow fluorescent protein (YFP) were transfected to 293T cells individually or simultaneously. As shown in FIG. 9H, YFP signal can be only detected when GREM1 and FGFR1 plasmids were co-transfected. In line with that, confocal microscopic imaging of immunofluorescent staining showed co-localization of Gremlin1 and FGFR1 on the membrane of LNCaP-R cells (FIG. 9I). In addition, soluble FGFR1 could compete the binding between Gremlin1 and FGFR1, revealed by a competitive ELISA experiment (FIG. 9G). The activation of the FGFR1/MEK/ERK signaling pathway due to Gremlin1 could be attenuated by applying excessive amount of soluble FGFR1 (FIG. 9G). These data provided strong supporting evidence for a specific binding between Gremlin and FGFR1, and uphold the notion that this binding was required for the activation of FGFR1 and its downstream MAPK signaling (FIG. 9G).

To further delineate the mode of Gremlin1/FGFR1 interaction, we performed co-immunoprecipitation between truncated FGFR1 (FIG. 9J-9K) and Gremlin1 or the classic FGFR1 ligand FGF1. It has been well defined that the extracellular region of FGFR1 is comprised of domain 1 (D1), domain 2 (D2) and domain 3 (D3). Consistently with previous reports that the linker between D2 and D3 is the key binding area between FGF1 and FGFR1, we found that loss of D2 or D3 abolished the co-immunoprecipitation of FGF1 and FGFR1. In contrast, only D2 missing abrogated the association between Gremlin1 and FGFR1, suggesting that Gremlin1 binds to FGFR1 at D2 (FIG. 9J-9K). Moreover, we mutated the previously identified key amino acid residues of FGFR1 (C176 and R248) in its binding pocket to FGF1 (FIG. 9P). As expected, FGFR1-C176G or FGFR1-R248Q indeed disrupted co-immunoprecipitation of FGF1 and FGFR1, whereas these two mutations did not affect the interaction between Gremlin1 and FGFR1 (FIG. 9P-9Q). These data indicated that FGFR1 binds to Gremlin1 in a way distinct from the binding mode with its classic ligand FGF1. In agreement with that, Fortebio assays, co-immunofluorescent staining and co-immunoprecipitation experiments together demonstrated that addition of Gremlin1 did not compete the association of FGF1 and FGFR1, and vice versa (FIG. 9R-9U). Therefore, Gremlin1 and FGF1 probably bind at different sites of FGFR1. We then assessed the expression levels of Gremlin1 and various FGFs in CRPC patient samples from published RNA-seq dataset (Robinson et al., Cell 161(5), 1215-1228 (2015)). We observed that the GREM1 transcript was higher than FGFs. These data collectively suggested that Gremlin1 serves as a major ligand for FGFR1 in CRPC.

The next question was to decipher the structural basis of Gremlin1/FGFR1 interaction. We used the HDOCK platform (http://hdock.phys.hust.edu.cn/) to perform docking of the previously characterized protein structures of Gremlin1 (PDB:5AEJ) (Kisonaite et al. Structure of Gremlin-1 and analysis of its interaction with BMP-2. Biochem J 473, 1593-1604(2016)) and FGFR1 extracellular region (PDB: 30JV) (Beenken et al. Plasticity in interactions of fibroblast growth factor 1 (FGF1) N terminus with FGF receptors underlies promiscuity of FGF1. J Biol Chem 287, 3067-3078 (2012)). As shown in FIG. 9B, two positively charged clusters of amino acid residues in Gremlin1 (Lys66-Arg67; R92-Lys123-Lys124-Arg148-Lys150-Arg153 relative to SEQ ID NO: 69) and extracellular domain 2 of FGFR1 were predicted to be an essential binding area between Gremlin1 and FGFR1. We then performed mutagenesis as described in FIGS. 9L and 9N to test the protein binding simulation. Lys123Lys124 to Ala123 Ala124 mutations on Gremlin1 (the numbering is relative to SEQ ID NO: 69) or E160A mutants of FGFR1 severely impaired the co-immunoprecipitation between Gremlin1 and FGFR1 (FIGS. 9M and 9O). Docking module highlights the key amino acid residues in the binding pocket between Gremlin1 and FGFR1 (FIG. 9V). Thus, Lys123-Lys124 of Gremlin1 (the numbering is relative to SEQ ID NO: 69) and corresponding Glu160 of FGFR1 were key amino acid residues for the formation of the Gremlin1/FGFR1 protein complex.

### Example 6: Anti-GREM1 Antibody Profoundly Inhibits the Castration-Resistant PCa Development in a Pbsn-Cre4; PTEN/f; Trp53/f GEMM

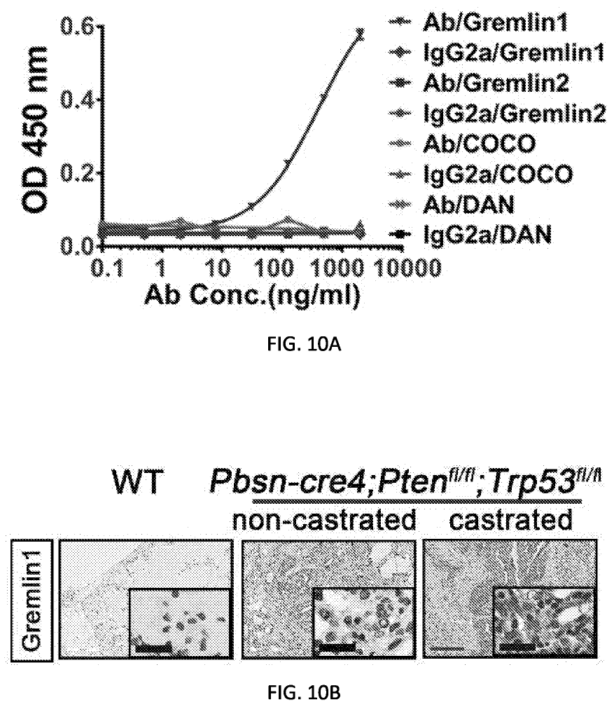

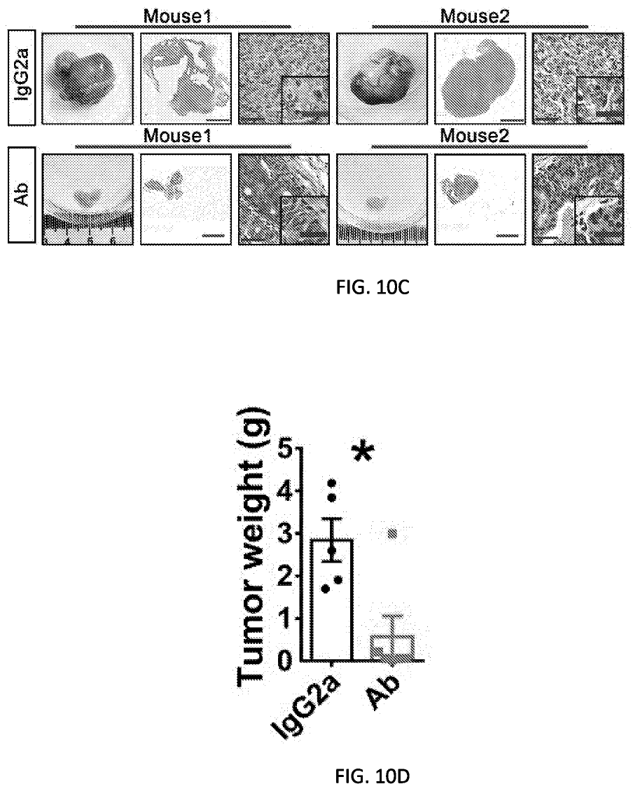

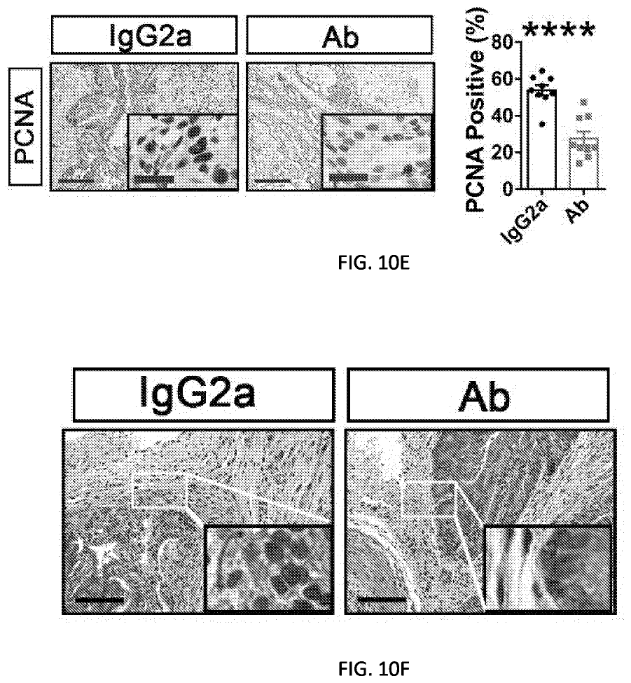

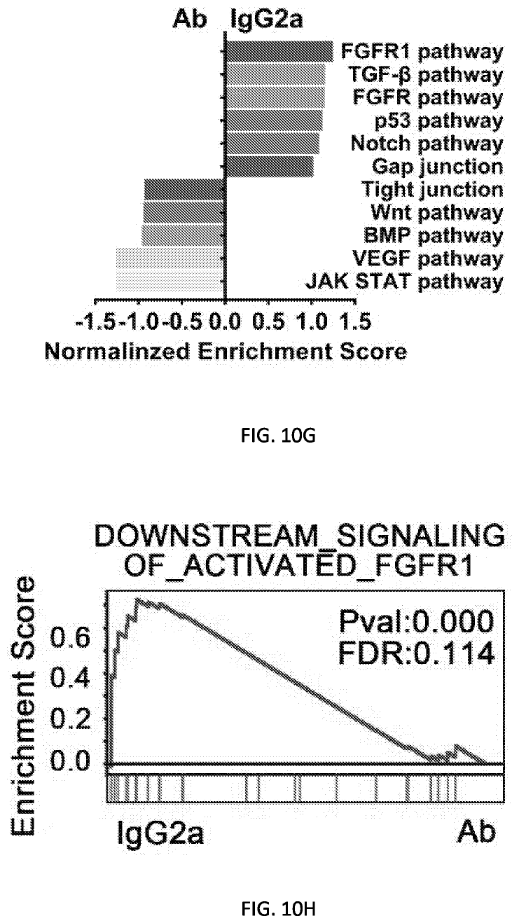

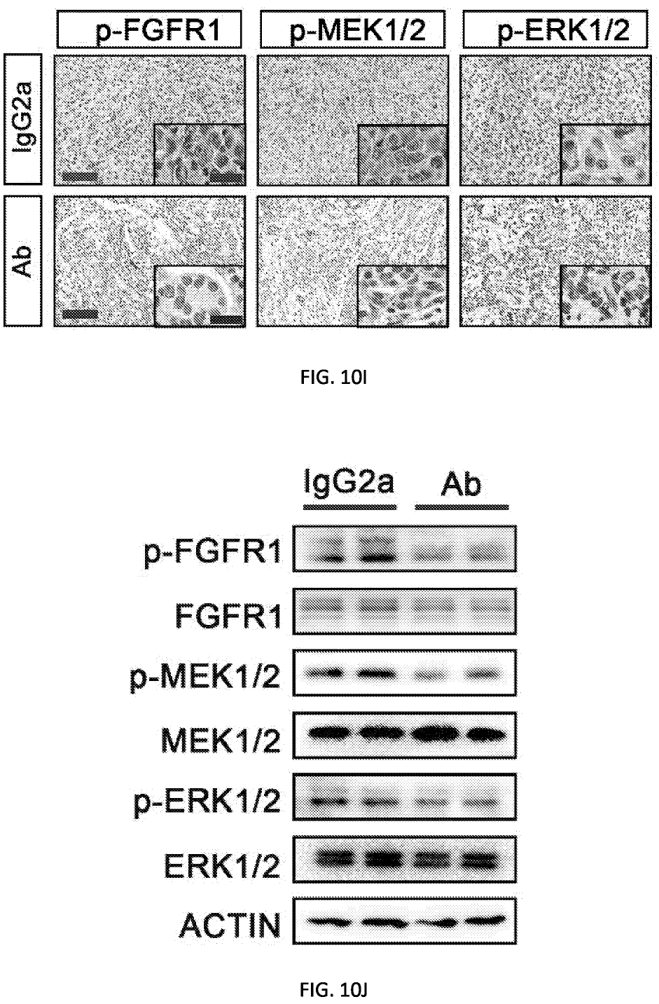

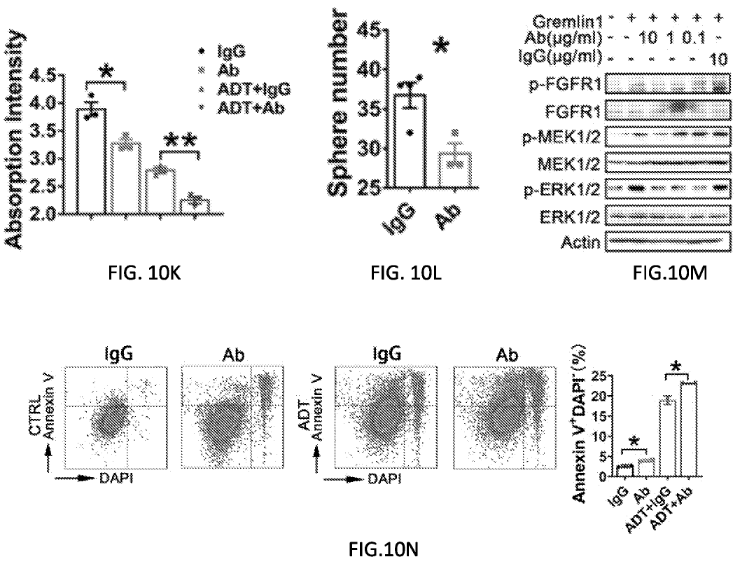

The upregulation and oncogenic effect of GREM1 in CRPC made it a promising therapeutic target. FIG. 10O shows schematics illustrating the treatments in a Pbsn-Cre4; Pten^{fl/fl}; Trp53^{fl/fl} GEMM. Mice which were castrated at 2 months received anti-mouse Gremlin1 antibody (anti-mGREM1 antibody) (i.p., 10 mg/kg) or IgG, as indicated, three times a week for 2 months. To target GREM1, we developed a monoclonal antibody against murine GREM1 with high affinity, which is also called a surrogate antibody in the present disclosure. This surrogate antibody did not bind to Gremlin2 or other BMP antagonists such as COCO and DAN (FIG. 10A). To test the effect of anti-mGREM1 antibody on PCa, we utilized a Pbsn-Cre4; PTEN^{fl/fl}; Trp53^{fl/fl} GEMM which developed spontaneous invasive PCa at an average of 3 months. It has been demonstrated previously that Pbsn-Cre4; PTEN^{fl/fl}; Trp53^{fl/fl} PCas are castration-resistant de novo. We quantified the expression levels of GREM1 in intact or castrated Pbsn-Cre4; PTEN^{fl/fl}; Trp53^{fl/fl} PCa and wild-type prostate samples by immunostaining. As shown in FIG. 10B, GREM1 was highly upregulated in castrated Pbsn-Cre4; PTEN^{fl/fl}; Trp53^{fl/fl} tumors followed by intact Pbsn-Cre4; PTEN^{fl/fl}; Trp53^{fl/fl} tumors compared to wild-type prostates, and was further enriched in castrated Pbsn-Cre4; PTEN^{fl/fl}; Trp53^{fl/fl} tumors.

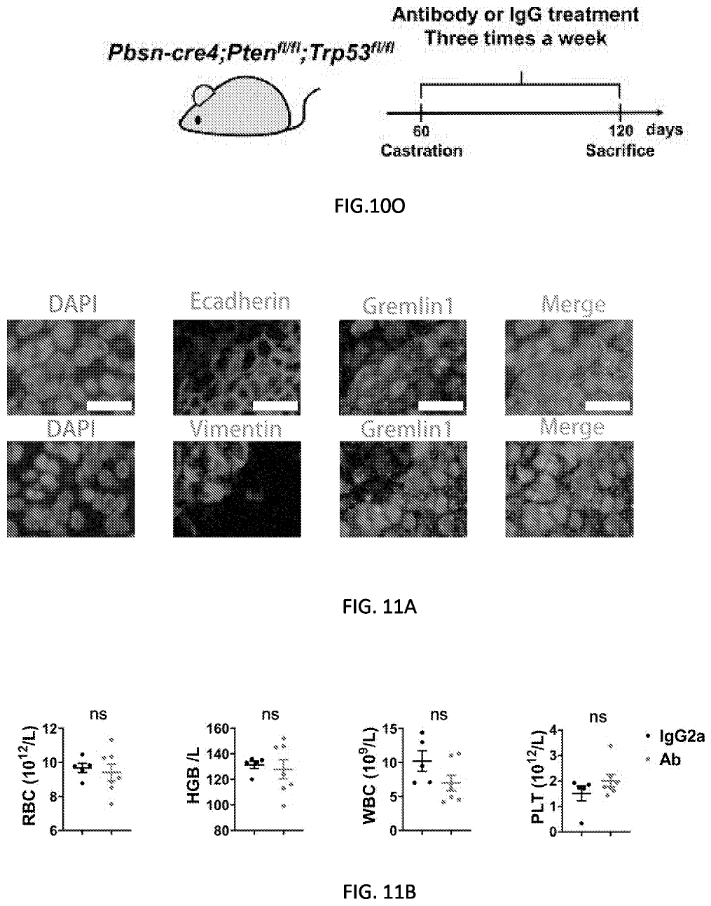

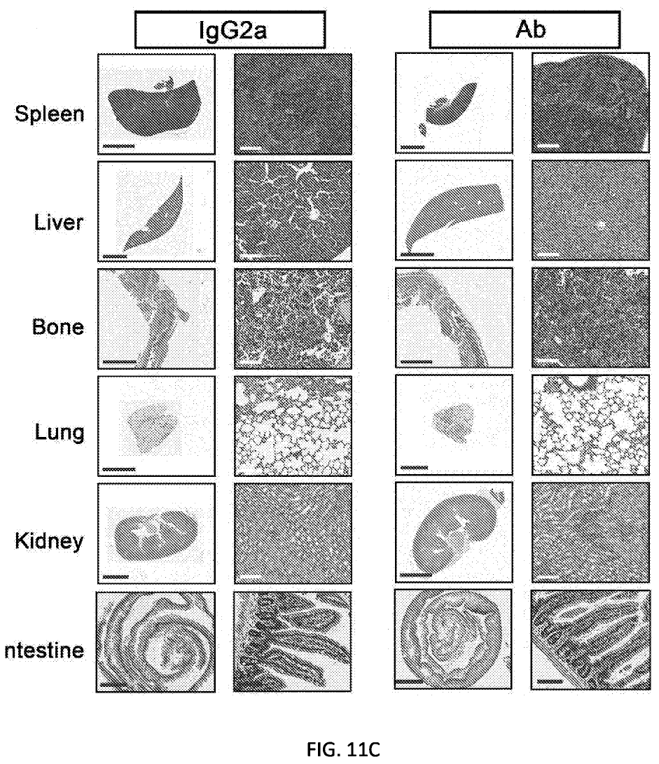

Coimmunostaining of Ecadherin and GREM1 suggested that Gremlin was largely expressed by the tumorous epithelial cells in castrated Pbsn-Cre4; PTEN^{fl/fl}; Trp53^{fl/fl} tumors (FIG. 11A). Notably, quantitative PCR analysis of Grem1 in several major organs of Pbsn-Cre4; PTEN^{fl/fl}; Trp53^{fl/fl} mice demonstrated that expression of Grem1 was highest in the prostate cancer tissue (FIG. 1I), which made Gremlin1 a plausible therapeutic target for PCa.

We applied the anti-mGREM1 antibody on castrated Pbsn-Cre4; PTEN^{fl/fl}; Trp53^{fl/fl} mice and evaluated its in vivo effect on CRPC development. Two-month-old PTEN/P53^{Δ/Δ} mice were castrated and subjected to anti-GREM1 antibody or control IgG2a treatment three times a week at 10 mg/kg for 8 weeks (FIG. 10C). Two months after the castration, animals were terminated for analysis. As shown in FIG. 10C, 10D, all control IgG2a treated mice developed aggressive CRPC. The anti-mGREM1 antibody exerted a profound repressive effect on PCa growth as evidenced by marked suppression of gross tumor appearance, tumor weight, and a significant reduction in proliferative PCNA positive cells (FIG. 10E). The H&E staining of prostate sections from anti-GREM1 treated Pbsn-Cre4; PTEN^{fl/fl}; Trp53^{fl/fl} mice showed mostly intraductal hyperplasia with intact basement membrane, which stood in great contrast to the invasive PCa phenotype in IgG2a injected mice (FIG. 10F). At the dosage of 10 mg/kg, we observe no evident distinctions in major organs including the intestine, lung, liver, spleen, bone marrow and kidney, nor alterations in peripheral blood cell count between the two experimental groups (FIG. 11B, 11C), suggesting that the profound anti-tumor effect of the anti-mGREM1 antibody was not accompanied by obvious side effects.

In order to understand the mechanism of the powerful inhibitory impact of anti-mGREM1 antibody on CRPC in mice, we carried out RNA-sequencing on prostate samples from IgG2a or anti-mGREM1 antibody treated mice. KEGG and GESA analysis demonstrated that FGFR and MAPK signaling were the most significantly changed signaling pathway in the anti-mGREM1 antibody treated group (FIG. 10G, 10H). Further immunostaining and immunoblotting experiments substantiated that the administration of anti-mGREM1 antibody resulted in notable decreases of FGFR1, MEK and ERK phosphorylation (FIG. 10I, 10J). These results collectively suggested that the anti-mGREM1 antibody suppressed CRPC development in Pbsn-Cre4; PTEN^{fl/fl}; Trp53^{fl/fl} mice through inhibition of the FGFR1/MAPK signaling pathway.

### Example 7: Antibody Targeting GREM1 Exerts a Strong Anti-Tumor Effect in Human PCa Cell Lines

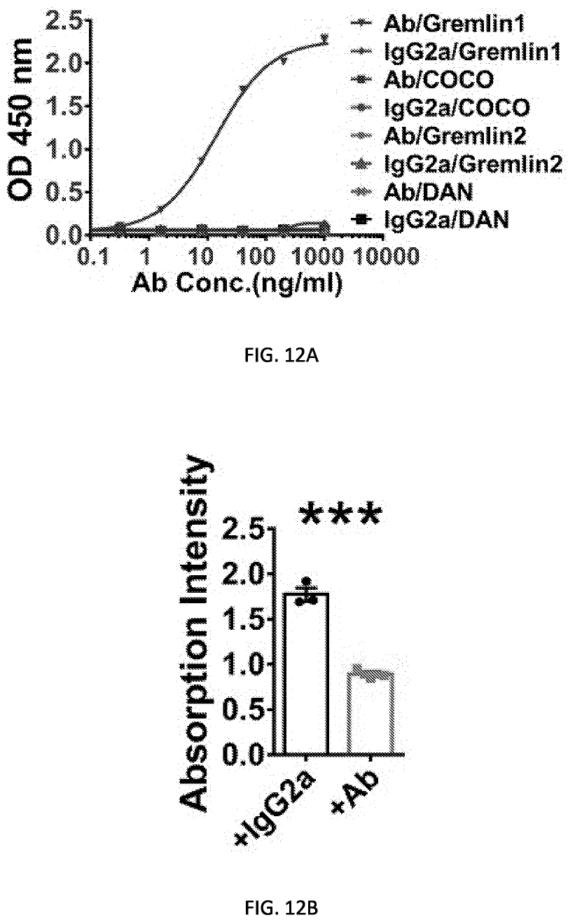

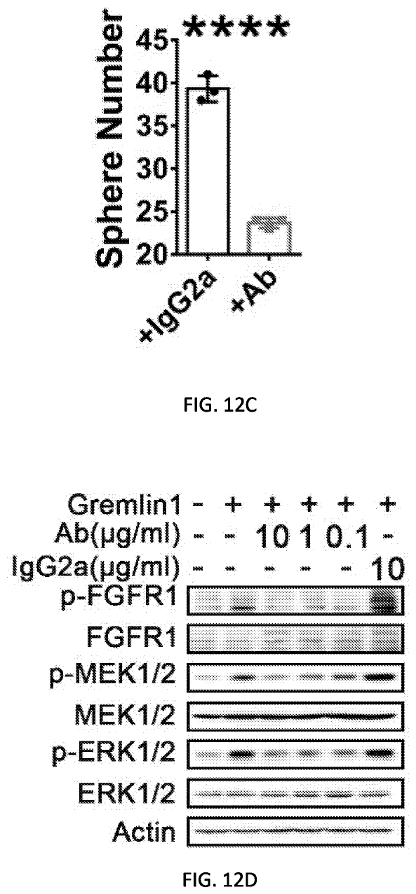

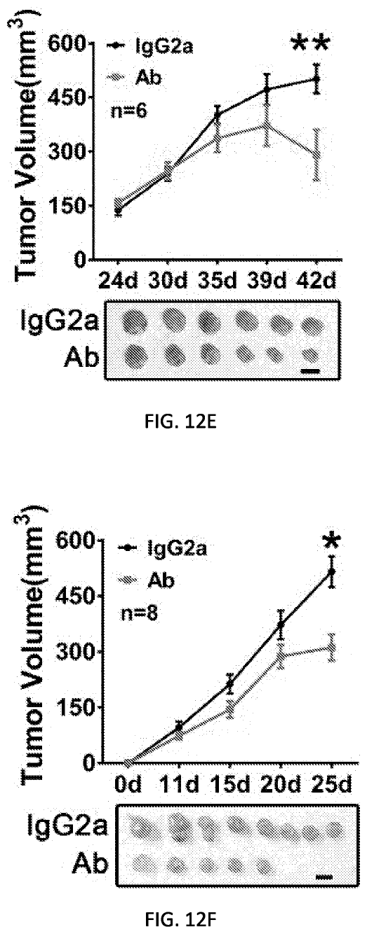

14E3 was tested to target GREM1 in human PCa. The affinity and specificity of this antibody to hGREM1 was verified by ELISA (FIG. 12A). Anti-human-GREM1 antibody 14E3 exerted an inhibitory impact on sphere formation, proliferation, and induced apoptosis of LNCaP and PC3 cells. This antibody potentiated the anti-tumor effect of Enzalutamide in AR dependent LNCaP cells (FIG. 12B, 12C, 10K, 10L). Biochemical analysis demonstrated a dose-dependent inhibition of FGFR1/MEK/ERK signaling pathway by anti-GREM1 antibody treatment in both PC3 and LNCaP cells (FIG. 12D, 10M).

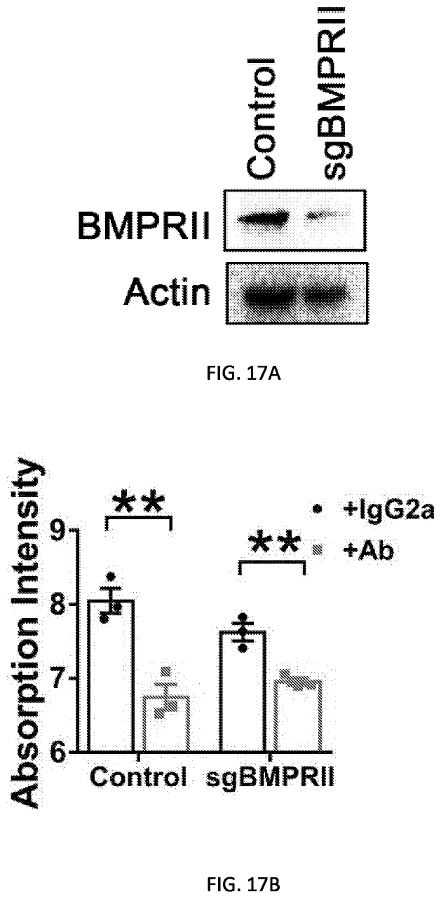

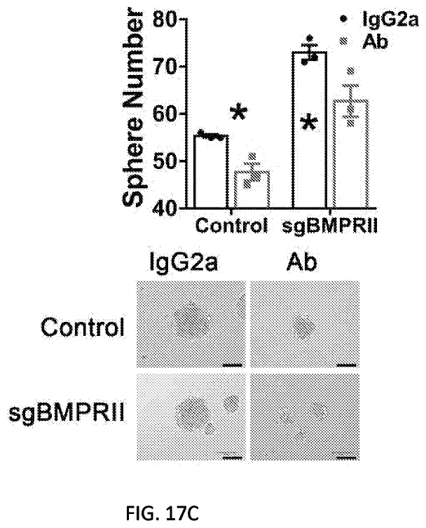

To investigate whether BMP signaling was involved in the anti-tumor effect of 14E3, BMPRII was knockout by CRISP/Cas9, which did not abrogate the inhibitory effect of 14E3 on PCa cells, indicating a BMP signaling was independent role of the anti-GREM1 antibody (FIGS. 17A, 17B and 17C).

To test the effect of 14E3 in CRPC in vivo, nude mice bearing the CRPC cell line PC3 xenograft were injected intraperitoneally with anti-GREM1 antibody or IgG2a three times a week for 2 weeks (10 mg/kg body weight). 14E3 greatly prohibited PC3 xenograft tumor growth. The tumor inhibition effected by 14E3 became more pronounced during secondary tumor passages (FIG. 12E, 12F).

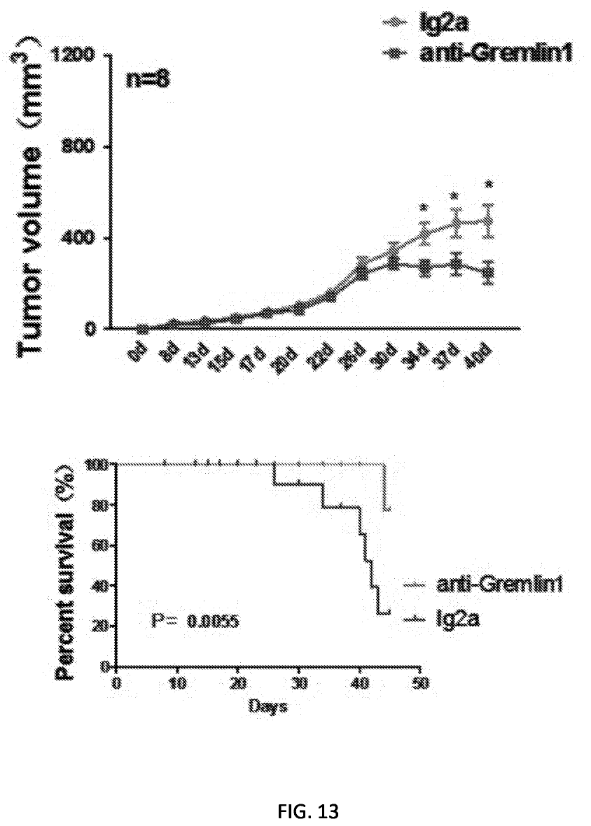

Tumor growth inhibition activity of 14E3 in PC3 CRPC model with castration was studied. Briefly, PC3 cells were subcutaneously implanted into Balb/c nude mice at 1×10⁶ cells per mouse and then mice were treated with castration surgery to make CRPC (castration-resistant prostate cancer) model. When tumor volume grew to 100 mm\hat{ }3 (Day 20), mice were treated with either isotype control mouse IgG2a or 14E3 hybridoma antibody (mlgG2a). Each group had 8 mice and antibodies were given intraperitoneally (i.p.) at 10 mg/kg twice a week. Tumor volume was measured twice a week in two dimensions using a caliper (INSIZE). The FIG. 13 showed that Gremlin antibody 14E3 reduced the growth of PC3 tumor and prolonged the survival of mice bearing the tumor.

### Example 8: Antibody Against Gremlin1 Exerts a Strong Anti-Tumor Effect in LNCaP PCa Cells

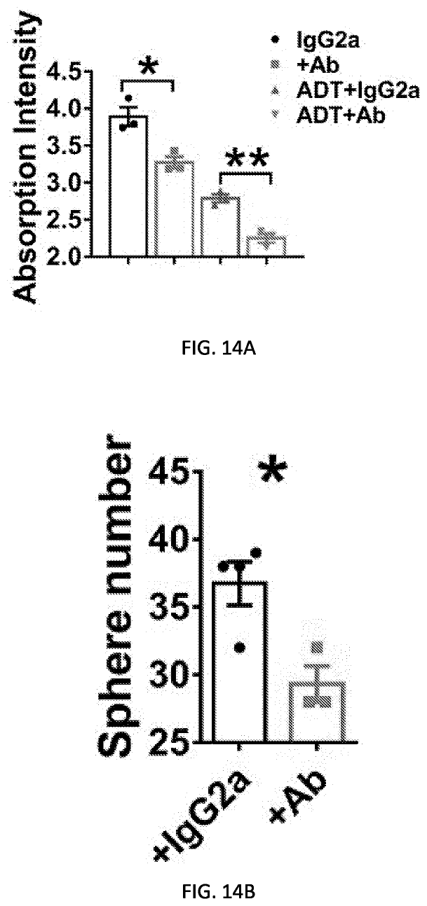

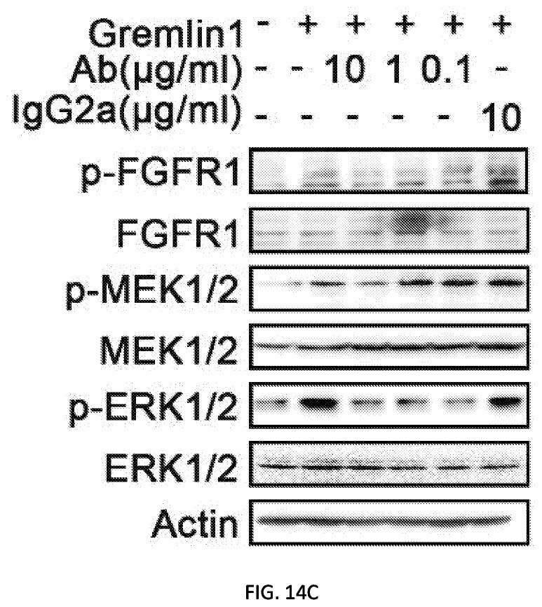

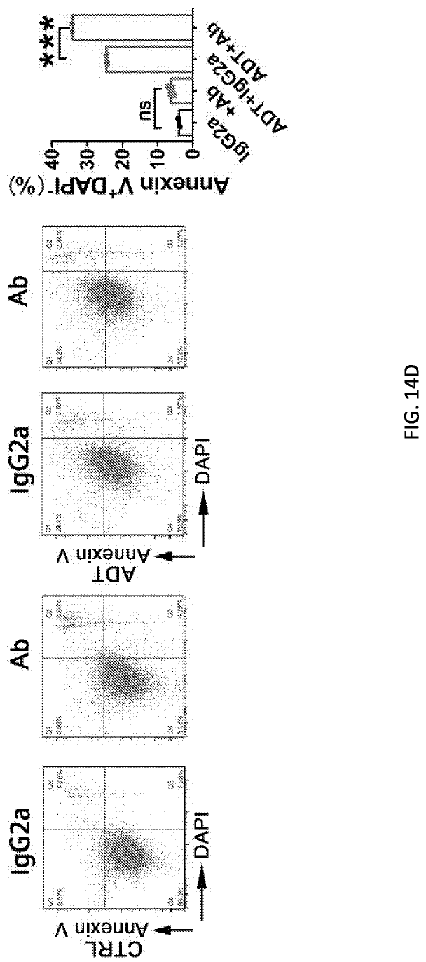

14E3 was tested to target GREM1 in LNCaP PCa cells. FIG. 14 shows that 14E3 exerted an inhibitory impact on sphere formation and proliferation, and potentiated the anti-tumor effect of Enzalutamide in AR dependent LNCaP cells (FIG. 14A,B). Biochemical analysis demonstrated a dose-dependent inhibition of FGFR1/MEK/ERK signaling pathway by 14E3 treatment in LNCaP cells (FIG. 14C). FIGS. 14D and 10N showed synergistic effects of the combination therapy of anti-GREM1 antibody (e.g., 14E3 or anti-mGREM1 antibody) and enzalutamide on tumor inhibition of CRPC. These data demonstrated that antibody targeting GREM1 can be served as a promising therapeutic approach for human CRPC patients.

### Example 9: 14E3 Inhibited GREM1 Mediated Tumor Cell Migration

PC-3 cells at log-growth phase were harvested and re-suspended in cell culture medium (DMEM medium supplied with 10% FBS). Cells were non-treated or treated with 1 μg/ml Gremlin or 10 μg/ml 14E3 or 10 μg/ml control mIgG2a for 3 days. Then cells were planted at 10⁵ cells/well in 6-well cell culture plate. After cells reached 100% coverage of the bottom of wells, the medium was changed to serum free medium. Each well was made with one scratch using 200 μl-tip. The migration rate of cells were analyzed by calculating the area of cells growing on the scratch using Image J software.

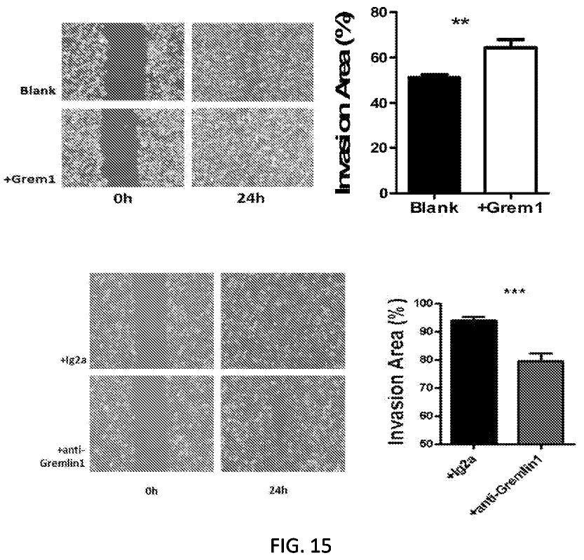

As shown in FIG. 15, addition of GREM1 accelerated cell migration of prostate cancer cells, and 14E3 reduced such acceleration, i.e., inhibited GREM1 mediated tumor cell migration.

### Example 10: 14E3 Inhibited GREM1 Induced EMT/Stem Cell Formation in LNCaP and PC3 Suspension Culture

In order to assess the role of gremlin1 in modulating tumor prostate cell growth, we tested the antibodies provided herein in an assay involving prostate cancer cell LNCaP. This cell line was transfected with a PSA promoter driven GFP expression lentivirus plasmid. PSA is known as a differentiated marker of prostate cell and prostate cancer cells with low level of PSA represent poorly differentiated or undifferentiated prostate cancer cells. These cells usually have stem cell like property and have more aggressive growth property. The LNCaP reporter cell assay is briefly described below.

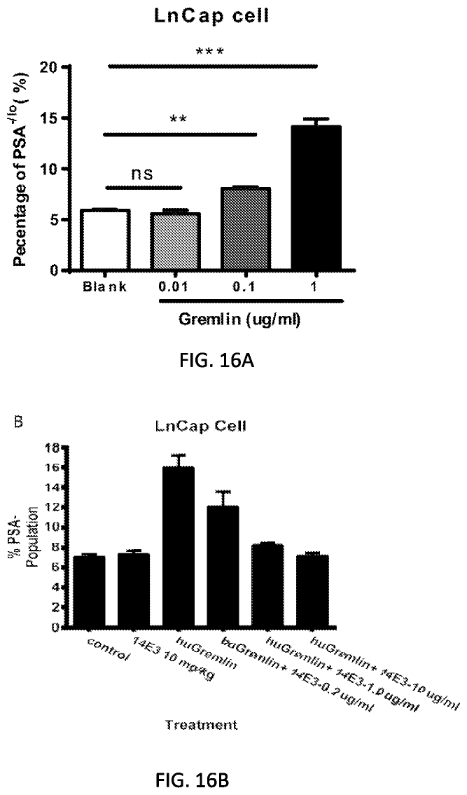

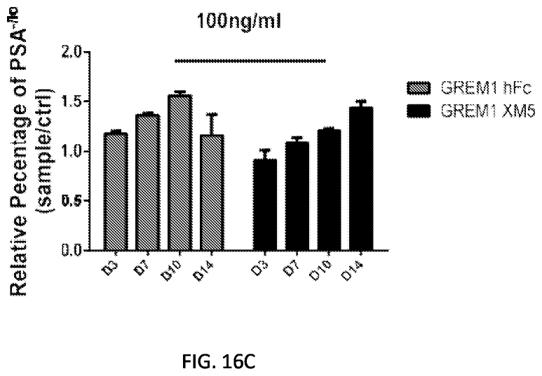

LNCaP-PSA cells were plated in 24-well plates at 10000/well in RPMI 1640/10% FBS (GIBCO), 1% P/S (complete media) and incubated at 37° ° C. and 5% CO₂ overnight. The next day, remove media, 1 ug/ml human gremlin (ACRO) or human gremlin with serially diluted antibodies were added to the cells. Change medium every three days. On day 7, remove media from the wells, wash with PBS twice, run flow cytometer (Bechman) using FITC channel. As shown in FIG. 16A, the percentage of the PSA-low LNCaP population in the control well (blank) was around 6%, and the percentage increased as the addition of GREM1 in a dose dependent manner, suggesting that GREM1 promoted the aggressiveness of the prostate cancer cells. The hybridoma antibody 14E3 provided herein neutralized the gremlin-mediated increase in PSA-low LNCAP population in a dose-dependent manner as shown in FIG. 16B, indicating that 14E3 was capable of reversing the GREM1-promoted aggressiveness.

FIG. 16C showed that substitution of the BMP-binding loop with a non-BMP-binding loop did not affect the GREM1 mediated increase in the percentage of PSA-1º cell population in prostate cancer cell (LNCaP). This suggested that GREM1 promoted cancer cell aggressiveness was independent of the BMP-binding loop on GREM1.

### Example 11: Effect of Anti-GREM1 Antibody in Suppressing Growth of Organoid Derived from Human CRPC Patients

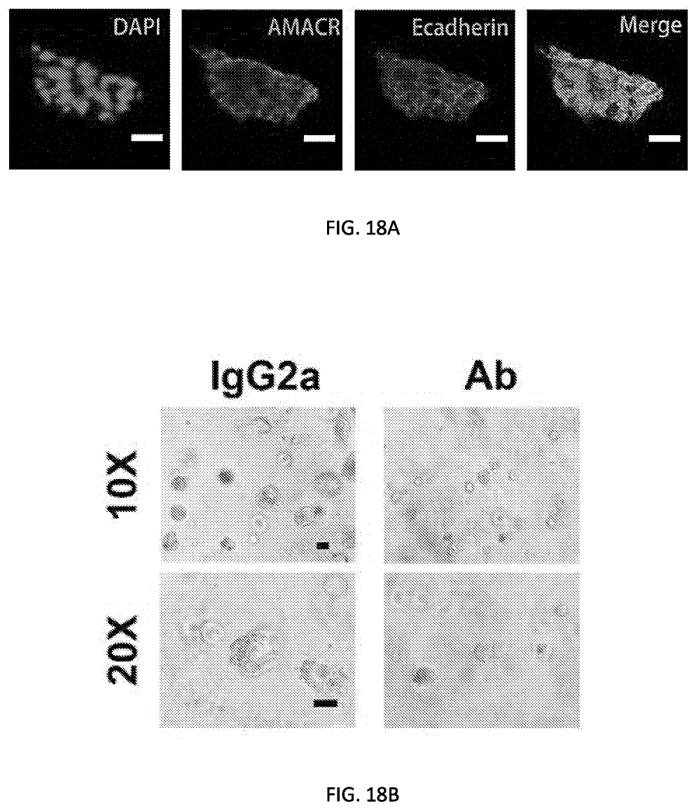

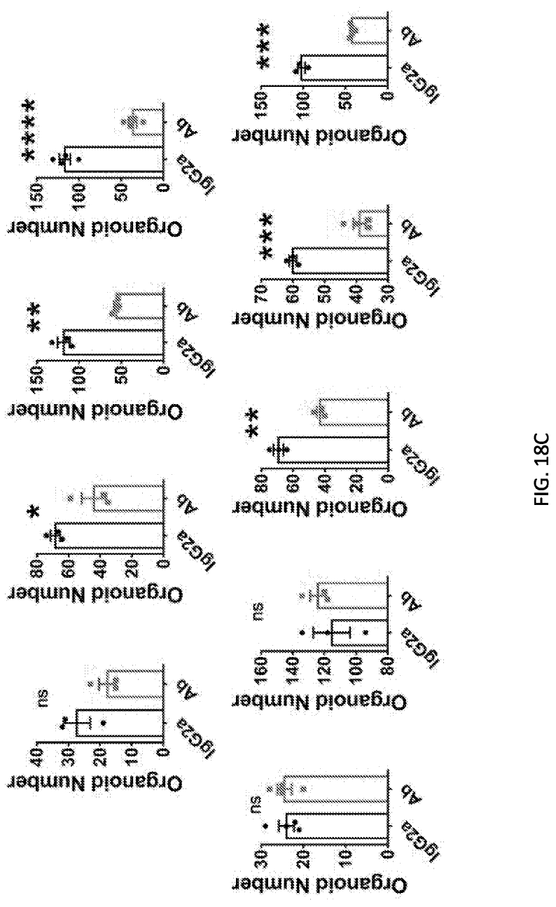

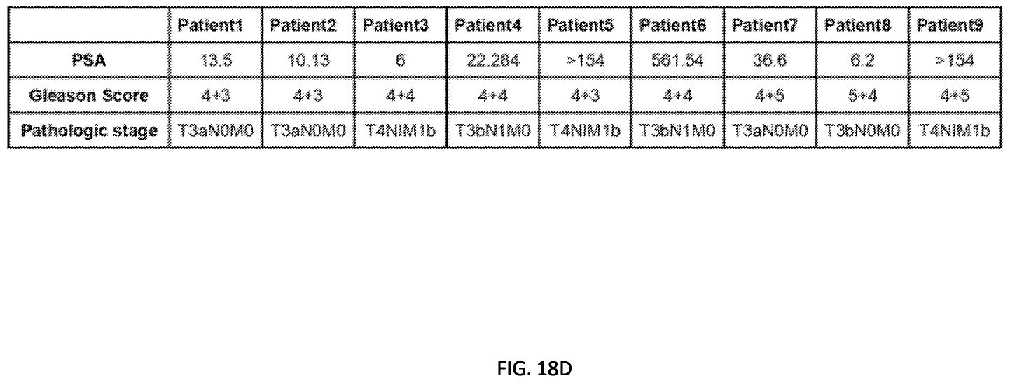

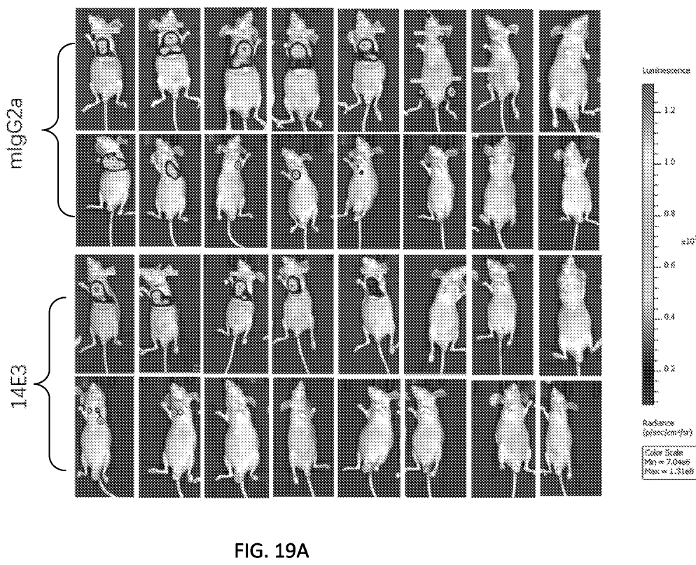

We then tested whether the anti-GREM1 antibody display suppressive effect on patient derived organoids (PDOs). The PDOs were freshly collected from PCa patients at Ren Ji Hospital. Briefly tumor tissues from nine CRPC patients were harvested from surgery and cut into 1-5 mm3 and washed once with HBSS, the tissues was then digested to single cell suspension using CollagenaseII+10 μM Y-27632 at 37° ° C. for 4 hours, followed by neutralization of digestion using base culture medium. After that the tumor tissue was further digested using 1 ml TrypLE+10 μM Y-27632 for 15 minutes at 37° C. followed by neutralization using medium with 10% FBS. The resulting cells were resuspended in 50% matrigel+50% medium and 50 ul of the cell suspension was dispensed into each well of the 96-well plate. Afterward the prewarmed PDO medium (B27, N acetylcysteine, EGF, Noggin, R-sponsdin 1, A83-1, FGF10, FGF2, Prostaglandin E2, Nicotinamide, SB202190, DHT and Y27632) was added to the culture and fresh medium was added every 2-3 days. FIGS. 18A, 18B, 18C and 18D show that 14E3 reduced the growth of organoid in 7 out of the 9 PDOs to different degree, demonstrating a potential mechanism for 14E3 mediated tumor growth inhibition and points to the therapeutic potential of gremlin based inhibition.

### Example 12: Discussion

Upon the widely application of the second-generation ADT drugs for PCa, the number of AR-independent CRPC has increased significantly. In this study, we find that the expression of GREM1 in CRPC is abnormally increased compared to hormone naïve or the newly diagnosed PCas. GREM1 promotes prostate cancer progression and tolerance to androgen deprivation. Those effects are achieved through a direct GREM1-FGFR binding to activate the FGFR/MAPK signaling pathway. The GREM1 blocking antibody can effectively inhibit castration-resistant growth of PCa in GEMM murine PCa models, human PCa cell lines, patient derived organoids and xenografts. These results together strongly support that the GREM1/FGFR1/MAPK signaling axis promotes PCa progression and point to Gremlin as an important and promising therapeutic target.

Second-generation anti-androgen drugs have been shown to trigger upregulation of key drivers for AR-independent CRPC. However, the mechanism by which these drivers are modulated by AR signaling remains incompletely understood. We find that GREM1 is negatively correlated with the AR signaling pathway in CRPC patient samples. AR activation or overexpression leads to a strong decrease of GREM1 expression in PCa cells. Conversely, GREM1 transcription markedly increases when AR is knockout or inhibited by enzalutamide. CHIP and luciferase reporter assays data together support that the suppression of GREM1 is achieved through binding of AR in the GREM1 promoter region for transcriptional suppression. These results suggest that the gene expression of GREM1, as a potent driver of CRPC, is transcriptionally inhibited by AR. Mechanisms of castration resistance development in PCa can be summarized into two major categories, 1) reactivate the AR signaling pathway through AR amplification, mutation or alternative splicing, or upregulation of glucocorticoid receptor (GR), 2) activation of alternative signaling pathway such as FGF, PRC1, BCL2 for AR-independent tumor growth and escape of cell death. We find that in the prostate cancer cell line with GREM1 overexpression, the FGFR/MAPK signaling pathway is abnormally activated. This is particularly relevant in the light of recent findings that the FGF signal activation is an essential molecular signature of AR-independent CRPC and is required for the AR-independent growth of CRPC. The implication of FGF-FGFR1 signal axis in the bone metastasis of prostate cancer has also been reported. In this current study, we find that GREM1 causes FGFR1 phosphorylation and activation in a concentration-dependent manner. FGFR1 phosphorylation induced by Gremlin is more durable than FGF1 stimulation. In addition, the RNA transcription abundance of GREM1 is higher than other FGFs in CRPC according to the sequencing data of the SU2C PCa cohort. Therefore, we propose that GREM1 is at least one of the leading causes to the abnormal activation of the FGFR1/MAPK signal in CRPC. Critically, using co-immunostaining, bi-FC, fortebio, co-IP, pull-down and computer simulation approaches, we provide compelling evidence that GREM1 can directly bind to FGFR1. Thus, the activation of FGFR1 induced by GREM1 is resulted from a direct ligand-receptor binding. GREM1 acts as a new ligand for FGFR1 in PCa.

GREM1 was considered as a classic antagonist of BMP before. The BMP signaling pathway and the downstream target gene were reported to significantly affect the progression and metastasis of PCa based on observations from conditional knockout mouse models. However, we find that BMP4 does not exert significant impact on the activation of FGFR/MAPK and the tumor-promoting effects on PCa by GREM1. In addition, the inhibitory impact of the GREM1 blocking antibody on PCa cells cannot be overridden by BMPRII knockout. Whereas, the positive role of GREM1 on PCa can be profoundly abolished by FGFR1 knockout. Collectively, activation of FGFR/MAPK and the CPRC-promoting effect of GREM1 is independent on the BMP signaling pathway. Our current work identifies a completely new function of GREM1 and adds a new member to the FGFR ligands. More broadly, the FGFR signal pathway is also pivotal oncogenic driver in other tumors such as bladder cancer, gastric cancer, lung cancer and breast cancer. Further study is warranted to understand whether GREM1/FGFR/MAPK axis is involved in the tumorigenesis and progression in other cancer types.

We find that GREM1 is expressed by tumor epithelial cells both in GEMM and human PCa samples by immunostaining. In line with that, it is reported by other independent labs that tumor cells or tumor stem cells highly express GREM1 in colon cancer and glioma. However, Julie B. Sneddon et al. analyzed the expression of GREM1 RNA in 774 different tumor cases and found that more than 50% of the tumor stromal cells are positive of GREM1 from colon, lung, pancreatic and breast cancer. Michael Quante et al. showed that Gremlin is significantly increased in tumor-associated fibroblasts (CAFs) in a gastric cancer model. The promoting effect of GREM1 on CRPC may not only act via an autocrine way on tumor cells, but also possibly through modulating tumor microenvironment to create a niche suitable for the growth and escape of cell death of PCa cells in harsh conditions, such as androgen deprivation.

Secreted protein is an important category of drug targets. In this study, we develop a monoclonal antibody against GREM1. Based on experiments on human PCa cell lines, PDO and PDX, as well as in vivo study on the Pbsn-Cre4; PTEN^{fl/fl}; Trp53^{fl/fl} murine PCa model, we demonstrate a prominent anti-tumor effect of the anti-GREM1 antibody. The anti-GREM1 antibody displays strong synergistic effect with ADT on PCa. However, we have to take into consideration that GREM1 is also expressed in other tissues. Study has shown that the conventional knockout of GREM1 in mice causes abnormal development of the intestinal tract and disorder of the hematopoietic system. In our study, we carefully examine the main organs including intestines of mice after anti-GREM1 antibody treatment. At a dose of 10 mg/kg through i.p. injection three times a week, we do not observe obvious toxic effects, nor significant damage to the main organs or peripheral blood cell counts. These observations suggest that a suitable dosing window can avoid unwanted side effects.

Most of the 24 antibody drugs approved by the FDA for the cancer therapy target immune checkpoint proteins or cell surface proteins in hematopoietic malignancies, the few others block HER2, EGFR, VEGF or VEGFR for the treatment of a small number of solid tumors. “Cold tumors” including PCa, with low CD8⁺ cytotoxic T cell infiltration, respond poorly to the immune checkpoint therapy. Therefore, it will be of great clinical importance to identify novel targets for new antibody-based drugs for “cold tumors”. Our finding that the GREM1/FGFR1/MAPK axis is a critical driver for CRPC not only provides insight for the understanding of molecular mechanisms of castration resistance development, but also demonstrates direct therapeutic relevance of GREM1 as a novel drug target. The anti-GREM1 monoclonal antibody holds great therapeutic promise for the treatment of CRPC.

### Example 13: 14E3 could Potently Reduce the Formation of PCa Metastases in the Lung

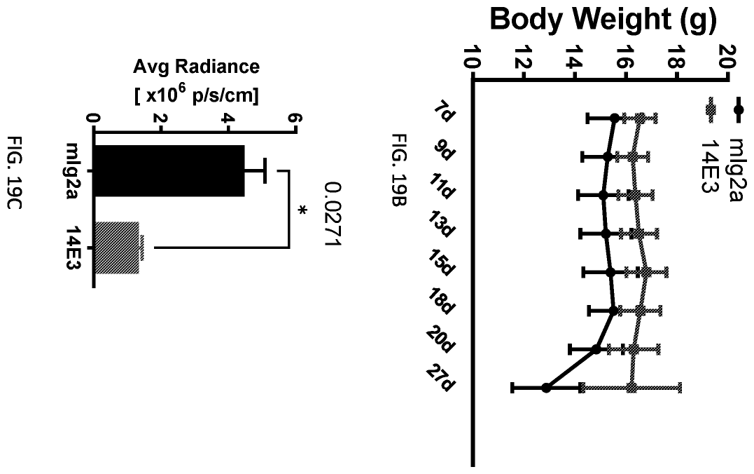

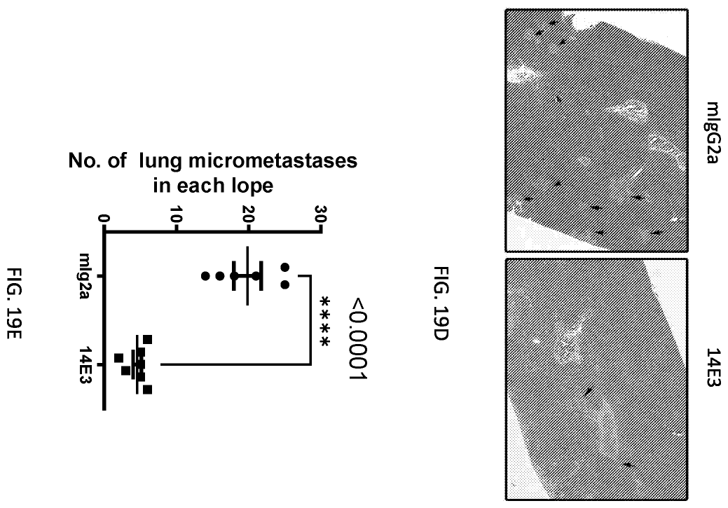

To evaluate the effect of gremlin1 antibody on prostate cancer metastases in the lung. PC3 cells (ATCC) were transfected with plasmid constitutively expressing luciferase. PC3-luc cells were collected from logarithmic phase of growth and suspended with 1×10⁶ cells in 80 ml basic media (DMEM). The cell suspension was then injected intracardiacally into BALB/C nude mice's heart ventricles (Shanghai SLAC Laboratory Animal). Gremlin1 hybridoma antibody 14E3 or isotype control was given intraperitoneally twice a week for three weeks at a dose of 10 mg/kg. Every two days, the body weight was measured. Mice were anesthetized and administered with D-luciferein (ThermoFisher, L2916) at 15 mg/kg for 5 mins. The Images were captured by the in vivo imaging system (Caliper IVIS bioluminescence system, Caliper LifeScience. USA). As a result of imaging in FIGS. 19A, 19B and 19C, 14E3 showed obvious inhibition on the average radiance intensity without influencing bodyweight, indicating that the antibodies against Gremlin1 (e.g., 14E3) significantly decreased the PCa metastasis in the mouse model subject to intracardiac injection. According to the statistics of micrometastasis in FIGS. 19D and 19E, 14E3 could potently reduce the formation of PCa metastases in the lung.

TABLE 5
Sequences mentioned or used in the present application

| SEQ | | |
|---|---|---|
| ID | | |
| NO | Sequence | Annotation |
| 1 | TYGMA | 14E3/Hu14E3-Ha/ |
| | | Hu14E3-Hb/ |
| | | Hu14E3-Hc HCDR1 |
| 2 | WINTLSGEPTYADDFKG | 14E3/Hu14E3-Ha/ |
| | | Hu14E3-Hb/ |
| | | Hu14E3-Hc HCDR2 |
| 3 | EPMDY | 14E3/Hu14E3-Ha/ |
| | | Hu14E3-Hb/ |
| | | Hu14E3-Hc HCDR3 |
| 4 | KSSQSLLDSDGKTYLS | 14E3/Hu14E3-La/ |
| | | Hu14E3-Lb LCDR1 |
| 5 | LVSKLDS | 14E3/Hu14E3-La/ |
| | | Hu14E3-Lb LCDR2 |
| 6 | WQGAHFPLT | 14E3/Hu14E3-La/ |
| | | Hu14E3-Lb LCDR3 |
| 7 | QIQLVQSGPELKKPGETVKISCKTSGSTFTTY | 14E3 VH |
| | GMAWMKQAPGKGLTWMGWINTLSGEPTY | |
| | ADDFKGRFAFSLKTSANTAYLQINNLKNED | |
| | AATYFCAREPMDYWGQGTSVIVSS | |
| 8 | DVVMTQTPLTLSITIGQPASISCKSSQSLLDS | 14E3 VL |
| | DGKTYLSWLLQRPDQSPKRLISLVSKLDSGV | |
| | PDRITGSGSGTDFTLKISRVEAEDLGIYYCW | |
| | QGAHFPLTFGAGTKLELK | |
| 9 | CAGATCCAGTTGGTACAGTCTGGACCTGA | 14E3 VHnu |
| | ACTGAAGAAGCCTGGAGAGACAGTCAAGA | |
| | TCTCCTGCAAGACTTCTGGATCTACGTTCA | |
| | CAACCTATGGAATGGCCTGGATGAAGCAG | |
| | GCTCCAGGAAAGGGTTTAACGTGGATGGG | |
| | CTGGATAAACACCCTCTCTGGAGAGCCAA | |
| | CATATGCTGATGACTTCAAGGGACGGTTT | |
| | GCCTTCTCTTTGAAAACCTCTGCCAACACT | |
| | GCCTATTTGCAGATCAACAACCTCAAAAA | |
| | TGAGGACGCGGCTACATATTTCTGTGCAC | |
| | GAGAACCAATGGACTACTGGGGTCAAGGA | |
| | ACCTCAGTCATCGTCTCCTCA | |
| 10 | GATGTTGTGATGACCCAGACTCCACTCACT | 14E3 VLnu |
| | TTGTCGATTACCATTGGACAACCAGCCTCC | |
| | ATCTCTTGCAAATCAAGTCAGAGCCTCTTA | |
| | GATAGTGATGGAAAGACATATTTGAGTTG | |
| | GTTGTTACAGAGGCCAGACCAGTCTCCAA | |
| | AGCGCCTAATCTCTCTGGTGTCCAAACTGG | |
| | ACTCTGGAGTCCCTGACAGGATCACTGGC | |
| | AGTGGATCAGGGACAGATTTCACACTGAA | |
| | AATCAGCAGAGTGGAGGCTGAAGATTTGG | |
| | GCATCTATTATTGCTGGCAAGGTGCACATT | |
| | TTCCGCTCACGTTCGGTGCTGGGACCAAGC | |
| | TGGAGCTGAAA | |
| 11 | DYYMN | 22F1/Hu22F1-Ha/ |
| | | Hu22F1-Hb/ |
| | | Hu22F1-Hc/Hu22F1- |
| | | Hd HCDR1 |
| 12 | DINPKDGDSGYSHKFKG | 22F1/Hu22F1-Ha/ |
| | | Hu22F1-Hb/ |
| | | Hu22F1-Hc/Hu22F1- |
| | | Hd HCDR2 |
| 13 | GFTTVVARGDY | 22F1/Hu22F1-Ha/ |
| | | Hu22F1-Hb/ |
| | | Hu22F1-Hc/Hu22F1- |
| | | Hd HCDR3 |
| 14 | KSSQSLLDSDGKTYLN | 22F1/Hu22F1-La/ |
| | | Hu22F1-Lb LCDR1 |
| 15 | LVSKLDS | 22F1/Hu22F1-La/ |
| | | Hu22F1-Lb LCDR2 |
| 16 | WQGTHFPYT | 22F1/Hu22F1-La/ |
| | | Hu22F1-Lb LCDR3 |
| 17 | EAQLQQSGPELVKPGASVKISCKASGYSFTD | 22F1 VH |
| | YYMNWLKQSHGKSLEWIGDINPKDGDSGYS | |
| | HKFKGKATLTVDKSSSTAYMELRSLTSEDSA | |
| | VYYCASGFTTVVARGDYWGQGTTLTVSS | |
| 18 | DVVMTQTPLTLSVTIGQPASISCKSSQSLLDS | 22F1 VL |
| | DGKTYLNWLLQRPGQSPKRLIYLVSKLDSGF | |
| | PDRFTGSGSGTDFTLKISRVEAEDLGVYYCW | |
| | QGTHFPYTFGGGTKLEIK | |
| 19 | GAGGCCCAGCTGCAACAATCTGGACCTGA | 22F1 VHnu |
| | ACTGGTGAAGCCTGGGGCTTCAGTGAAGA | |
| | TATCCTGTAAGGCTTCTGGATACTCGTTCA | |
| | CTGACTACTACATGAACTGGCTGAAGCAG | |
| | AGCCATGGAAAGAGCCTTGAGTGGATTGG | |
| | AGATATTAATCCTAAAGATGGTGATAGTG | |
| | GTTACAGCCATAAGTTCAAGGGCAAGGCC | |
| | ACATTGACTGTAGACAAGTCCTCCAGCAC | |
| | AGCCTACATGGAGCTCCGCAGCCTGACAT | |
| | CTGAGGACTCTGCAGTCTATTACTGTGCAA | |
| | GCGGATTTACCACGGTAGTAGCTAGGGGG | |
| | GACTACTGGGGCCAAGGCACCACTCTCAC | |
| | AGTCTCCTCA | |
| 20 | GATGTTGTGATGACCCAGACTCCACTCACT | 22F1 VLnu |
| | TTGTCGGTTACCATTGGACAACCAGCCTCC | |
| | ATCTCTTGCAAGTCAAGTCAGAGCCTCTTA | |
| | GATAGTGATGGAAAGACATATTTGAATTG | |
| | GTTGTTACAGAGGCCAGGCCAGTCTCCAA | |
| | AGCGCCTAATCTATTTGGTGTCTAAACTGG | |
| | ACTCTGGATTCCCTGACAGGTTCACTGGCA | |
| | GTGGATCAGGGACAGATTTCACACTGAAA | |
| | ATCAGCAGAGTGGAGGCTGAGGATTTGGG | |
| | AGTTTATTATTGCTGGCAAGGTACACATTT | |
| | TCCGTACACGTTCGGAGGGGGGACCAAGC | |
| | TGGAAATAAAA | |
| 21 | DDYMH | 69H5 HCDR1 |
| 22 | WIDPENGDTEYASKFQG | 69H5 HCDR2 |
| 23 | WATVPDFDY | 69H5 HCDR3 |
| 24 | KSSQSLLNRSNQKNYLA | 69H5 LCDR1 |
| 25 | FTSTRES | 69H5 LCDR2 |
| 26 | QQHYSTPFT | 69H5 LCDR3 |
| 27 | EVQLQQSGAELVRPGASVKLSCTASGFNIKD | 69H5 VH |
| | DYMHWVKRRPEQGLEWIGWIDPENGDTEY | |
| | ASKFQGKATITADTSSNTAYLQLSSLTSEDT | |
| | AVYYCTTWATVPDFDYWGQGTTLTVSS | |
| 28 | DIVMTQSPSSLAMSVGQKVTMSCKSSQSLL | 69H5 VL |
| | NRSNQKNYLAWYQQKPGQSPKLLVHFTSTR | |
| | ESGVPDRFIGSGSGTDFTLTISNLQAEDLADY | |
| | FCQQHYSTPFTFGSGTKLEIK | |
| 29 | GAGGTGCAGCTGCAACAGTCCGGCGCTGA | 69H5 VHnu |
| | ACTGGTGAGGCCTGGAGCCTCCGTGAAGC | |
| | TGTCCTGCACCGCCAGCGGCTTCAACATCA | |
| | AGGACGACTACATGCACTGGGTGAAGAGG | |
| | AGGCCTGAGCAGGGCCTGGAGTGGATCGG | |
| | CTGGATCGACCCCGAGAACGGCGACACCG | |
| | AGTACGCCTCCAAGTTCCAGGGCAAGGCC | |
| | ACCATCACCGCCGACACCTCCTCCAACAC | |
| | CGCCTACCTGCAGCTGAGCTCCCTGACCTC | |
| | CGAGGACACCGCCGTGTACTATTGCACCA | |
| | CCTGGGCCACCGTGCCCGACTTCGACTACT | |
| | GGGGACAGGGCACCACCCTGACCGTGTCC | |
| | AGC | |
| 30 | GATATCGTGATGACCCAGTCTCCTTCCTCT | 69H5 VLnu |
| | CTGGCTATGTCAGTGGGACAGAAAGTGAC | |
| | CATGTCTTGCAAGTCCTCTCAGTCTCTGCT | |
| | GAACAGGTCCAACCAGAAGAACTACCTGG | |
| | CTTGGTACCAGCAGAAACCAGGACAGTCT | |
| | CCTAAGCTGCTGGTGCATTTTACCTCTACC | |
| | AGGGAATCCGGAGTGCCAGATAGATTTAT | |
| | CGGCTCTGGCTCCGGCACAGATTTTACACT | |
| | GACCATCTCCAATCTGCAGGCAGAAGATC | |
| | TGGCTGACTACTTTTGCCAGCAGCACTACT | |
| | CCACCCCTTTTACCTTTGGCTCCGGCACCA | |
| | AGCTGGAGATCAAG | |
| 31 | DFYMN | 56C11 HCDR1 |
| 32 | DINPNNGGTSYNQKFKG | 56C11 HCDR2 |
| 33 | DPIYYDYDEVAY | 56C11 HCDR3 |
| 34 | RSSQSLVHSNGNTYLH | 56C11 LCDR1 |
| 35 | KVSNRFS | 56C11 LCDR2 |
| 36 | SQSTHVPLT | 56C11 LCDR3 |
| 37 | EVQLQQSGPELVKPGASVKISCKASGYTFTD | 56C11 VH |
| | FYMNWVKQSHGKSLEWIGDINPNNGGTSYN | |
| | QKFKGKATLTVDKSSSTAYMELRSLTSEDSA | |
| | VYYCARDPIYYDYDEVAYWGQGTLVTVSA | |
| 38 | DVVMTQTPLSLPVSLGDQASISCRSSQSLVH | 56C11 VL |
| | SNGNTYLHWYLQKPGQSPKLLIYKVSNRFS | |
| | GVPDRFSGSGSGTDFTLKISRVEAEDLGVYF | |
| | CSQSTHVPLTFGAGTKLELK | |
| 39 | GAGGTGCAGCTGCAGCAGTCCGGCCCTGA | 56C11 VHnu |
| | GCTGGTGAAGCCTGGAGCCTCCGTGAAGA | |
| | TCTCCTGTAAGGCCTCCGGCTACACCTTCA | |
| | CCGACTTCTACATGAACTGGGTGAAGCAG | |
| | TCCCACGGCAAGTCCCTGGAGTGGATCGG | |
| | CGACATCAATCCCAACAACGGCGGCACCT | |
| | CCTACAACCAGAAGTTCAAGGGCAAGGCC | |
| | ACCCTGACAGTGGACAAGTCCTCCAGCAC | |
| | CGCCTACATGGAGCTGAGGTCCCTGACCT | |
| | CCGAGGACTCCGCCGTGTACTACTGCGCC | |
| | AGGGACCCCATCTACTACGACTACGACGA | |
| | GGTGGCCTACTGGGGCCAGGGAACCCTGG | |
| | TGACAGTGTCCGCC | |
| 40 | GATGTGGTGATGACACAGACACCTCTGTC | 56C11 VLnu |
| | TCTGCCAGTGTCTCTCGGAGATCAGGCTTC | |
| | TATCTCTTGCAGATCCTCTCAGTCTCTGGT | |
| | GCATTCCAACGGAAACACCTACCTGCATT | |
| | GGTACCTGCAGAAACCAGGACAGTCTCCT | |
| | AAGCTGCTGATCTACAAGGTGTCCAACAG | |
| | GTTCTCCGGAGTGCCAGATAGATTTTCCGG | |
| | ATCTGGATCTGGCACCGATTTTACCCTGAA | |
| | GATCTCTAGAGTGGAAGCAGAGGATCTGG | |
| | GAGTGTACTTTTGTAGCCAGTCTACCCACG | |
| | TGCCTCTGACATTTGGAGCAGGAACAAAG | |
| | CTGGAGCTGAAG | |
| 41 | QVQLVQSGSELKKPGASVKVSCKASGYTFT | Hu14E3-Ha VH |
| | TYGMAWMRQAPGQGLEWMGWINTLSGEP | |
| | TYADDFKGRFVFSLDTSVSTAYLQISSLKAE | |
| | DTAVYYCAREPMDYWGQGTMVTVSS | |
| 42 | CAGGTGCAGCTGGTGCAGTCCGGCTCCGA | Hu14E3-Ha VHnu |
| | GCTGAAGAAGCCTGGCGCCTCCGTGAAGG | |
| | TGTCCTGCAAGGCCTCCGGCTACACCTTCA | |
| | CCACCTACGGCATGGCCTGGATGAGGCAG | |
| | GCTCCTGGCCAGGGACTGGAGTGGATGGG | |
| | CTGGATCAACACCCTGTCCGGCGAACCCA | |
| | CCTACGCCGACGACTTCAAGGGCAGGTTC | |
| | GTGTTCTCCCTGGACACCAGCGTGTCCACC | |
| | GCCTACCTGCAGATCTCCTCCCTGAAGGCC | |
| | GAGGACACCGCCGTGTACTACTGCGCCAG | |
| | GGAGCCCATGGACTACTGGGGCCAGGGCA | |
| | CCATGGTGACCGTGTCCTCC | |
| 43 | QIQLVQSGSELKKPGASVKVSCKASGYTFTT | Hu14E3-Hb VH |
| | YGMAWMRQAPGQGLEWMGWINTLSGEPT | |
| | YADDFKGRFAFSLDTSVSTAYLQISSLKAED | |
| | TAVYYCAREPMDYWGQGTMVTVSS | |
| 44 | CAGATCCAGCTGGTGCAGAGCGGCAGCGA | Hu14E3-Hb VHnu |
| | GCTGAAGAAGCCCGGCGCTAGCGTGAAGG | |
| | TGTCCTGCAAGGCCAGCGGCTACACCTTC | |
| | ACCACCTACGGCATGGCCTGGATGAGGCA | |
| | GGCTCCTGGACAGGGCCTGGAGTGGATGG | |
| | GCTGGATCAACACCCTGTCCGGCGAGCCT | |
| | ACCTACGCCGACGACTTCAAGGGCAGGTT | |
| | CGCCTTCTCCCTGGACACCTCCGTGAGCAC | |
| | CGCCTACCTGCAGATCTCCAGCCTGAAGG | |
| | CCGAGGACACCGCCGTGTACTACTGCGCC | |
| | AGGGAGCCTATGGACTACTGGGGCCAGGG | |
| | CACCATGGTGACCGTGTCCAGC | |
| 45 | QIQLVQSGSELKKPGASVKVSCKASGSTFTT | Hu14E3-Hc VH |
| | YGMAWMKQAPGQGLTWMGWINTLSGEPT | |
| | YADDFKGRFAFSLDTSVSTAYLQISSLKAED | |
| | TAVYYCAREPMDYWGQGTMVTVSS | |
| 46 | CAGATCCAGCTGGTGCAGTCCGGCAGCGA | Hu14E3-Hc VHnu |
| | GCTCAAGAAGCCCGGAGCCAGCGTGAAGG | |
| | TGTCCTGCAAGGCCAGCGGCTCCACCTTCA | |
| | CCACATACGGCATGGCCTGGATGAAGCAG | |
| | GCTCCTGGCCAGGGCCTGACCTGGATGGG | |
| | ATGGATCAACACCCTGTCCGGCGAGCCTA | |
| | CCTACGCCGATGACTTCAAGGGCAGGTTC | |
| | GCCTTCTCCCTGGACACCTCCGTGTCCACC | |
| | GCTTACCTGCAGATCTCCTCCCTGAAGGCC | |
| | GAGGACACCGCCGTGTACTACTGCGCCAG | |
| | GGAGCCCATGGACTACTGGGGCCAGGGCA | |
| | CCATGGTGACCGTGTCCTCC | |
| 47 | DVVMTQSPLSLPVTLGQPASISCKSSQSLLDS | Hu14E3-La VL |
| | DGKTYLSWLQQRPGQSPRRLIYLVSKLDSG | |
| | VPDRFSGSGSGTDFTLKISRVEAEDVGVYYC | |
| | WQGAHFPLTFGQGTKLEIK | |
| 48 | GATGTGGTGATGACACAGTCTCCTCTGTCT | Hu14E3-La VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTATCTGTCTT | |
| | GGCTGCAGCAGAGACCAGGACAGTCTCCT | |
| | AGAAGACTGATCTACCTGGTGTCCAAGCT | |
| | GGATTCTGGAGTGCCAGATAGATTTTCCG | |
| | GCTCCGGCTCTGGCACAGATTTCACCCTGA | |
| | AGATCTCTAGAGTGGAGGCAGAAGACGTG | |
| | GGAGTGTACTATTGTTGGCAGGGAGCTCA | |
| | CTTCCCTCTGACATTTGGACAGGGAACAA | |
| | AGCTGGAGATCAAG | |
| 49 | DVVMTQSPLSLPVTLGQPASISCKSSQSLLDS | Hu14E3-Lb VL |
| | DGKTYLSWLQQRPGQSPRRLISLVSKLDSGV | |
| | PDRFSGSGSGTDFTLKISRVEAEDVGVYYCW | |
| | QGAHFPLTFGQGTKLEIK | |
| 50 | GATGTGGTGATGACACAGTCTCCTCTGTCT | Hu14E3-Lb VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTATCTGTCTT | |
| | GGCTGCAGCAGAGACCAGGACAGTCTCCT | |
| | AGAAGACTGATCTCCCTGGTGTCTAAGCT | |
| | GGATTCCGGAGTGCCAGATAGATTTTCCG | |
| | GATCTGGATCTGGCACCGATTTTACCCTGA | |
| | AGATCTCTAGAGTGGAGGCAGAAGACGTG | |
| | GGAGTGTACTATTGTTGGCAGGGAGCTCA | |
| | CTTCCCTCTGACATTTGGACAGGGAACAA | |
| | AGCTGGAGATCAAG | |
| 51 | QVQLVQSGAEVKKPGASVKVSCKASGYSFT | Hu22F1-Ha VH |
| | DYYMNWVRQAPGQGLEWMGDINPKDGDS | |
| | GYSHKFKGRVTMTRDTSTSTVYMELSSLRS | |
| | EDTAVYYCASGFTTVVARGDYWGQGTTVT | |
| | VSS | |
| 52 | CAAGTTCAGCTGGTGCAGTCCGGAGCCGA | Hu22F1-Ha VHnu |
| | GGTGAAGAAGCCCGGCGCTTCCGTGAAGG | |
| | TGTCTTGTAAGGCCTCCGGCTACTCCTTCA | |
| | CCGATTACTACATGAACTGGGTGAGGCAA | |
| | GCTCCCGGTCAAGGTCTGGAGTGGATGGG | |
| | CGACATCAACCCCAAGGACGGCGACTCCG | |
| | GCTATTCCCACAAGTTCAAGGGTCGTGTG | |
| | ACCATGACCAGGGACACGTCCACCAGCAC | |
| | CGTGTACATGGAGCTGTCCTCTTTAAGGTC | |
| | CGAGGACACCGCCGTGTACTACTGCGCCA | |
| | GCGGATTCACCACCGTGGTGGCTAGGGGC | |
| | GACTATTGGGGCCAAGGTACCACCGTGAC | |
| | AGTGTCCAGC | |
| 53 | QVQLVQSGAEVKKPGASVKVSCKASGYSFT | Hu22F1-Hb VH |
| | DYYMNWVRQAPGQGLEWMGDINPKDGDS | |
| | GYSHKFKGRVTMTVDKSTSTVYMELSSLRS | |
| | EDTAVYYCASGFTTVVARGDYWGQGTTVT | |
| | VSS | |
| 54 | CAAGTTCAGCTGGTGCAGTCCGGAGCCGA | Hu22F1-Hb VHnu |
| | GGTGAAGAAGCCCGGCGCTTCCGTGAAGG | |
| | TGTCTTGTAAGGCCTCCGGCTACTCCTTCA | |
| | CCGATTACTACATGAACTGGGTGAGGCAA | |
| | GCTCCCGGTCAAGGTCTGGAGTGGATGGG | |
| | CGACATCAACCCCAAGGACGGCGACTCCG | |
| | GCTATTCCCACAAGTTCAAGGGTCGTGTG | |
| | ACCATGACCGTGGACAAGTCCACCAGCAC | |
| | CGTGTACATGGAGCTGTCCTCTTTAAGGTC | |
| | CGAGGACACCGCCGTGTACTACTGCGCCA | |
| | GCGGATTCACCACCGTGGTGGCTAGGGGC | |
| | GACTATTGGGGCCAAGGTACCACCGTGAC | |
| | AGTGTCCAGC | |
| 55 | QAQLVQSGAEVKKPGASVKVSCKASGYSFT | Hu22F1-Hc VH |
| | DYYMNWVRQAPGQGLEWMGDINPKDGDS | |
| | GYSHKFKGRVTLTVDKSTSTVYMELRSLRS | |
| | EDTAVYYCASGFTTVVARGDYWGQGTTVT | |
| | VSS | |
| 56 | CAAGCTCAGCTGGTGCAGTCCGGCGCTGA | Hu22F1-Hc VHnu |
| | GGTGAAAAAGCCCGGCGCCAGCGTGAAGG | |
| | TGTCTTGTAAGGCCTCCGGCTACTCCTTCA | |
| | CCGACTACTACATGAACTGGGTGAGGCAA | |
| | GCTCCCGGTCAAGGTCTGGAGTGGATGGG | |
| | CGACATCAACCCCAAGGACGGCGACAGCG | |
| | GCTACTCCCACAAGTTCAAGGGTCGTGTG | |
| | ACTTTAACCGTGGACAAGTCCACCTCCACC | |
| | GTCTACATGGAGCTGAGGTCTTTAAGGTCC | |
| | GAGGATACCGCCGTGTACTACTGCGCTAG | |
| | CGGCTTCACCACCGTGGTGGCTCGTGGCG | |
| | ATTACTGGGGACAAGGTACCACCGTGACC | |
| | GTGTCCTCC | |
| 57 | QAQLVQSGAEVKKPGASVKVSCKASGYSFT | Hu22F1-Hd VH |
| | DYYMNWLRQAPGQGLEWIGDINPKDGDSG | |
| | YSHKFKGRATLTVDKSTSTVYMELRSLRSE | |
| | DTAVYYCASGFTTVVARGDYWGQGTTVTV | |
| | SS | |
| 58 | CAAGCTCAACTGGTGCAGTCCGGCGCCGA | Hu22F1-Hd VHnu |
| | GGTGAAAAAGCCCGGTGCCTCCGTGAAGG | |
| | TGAGCTGCAAGGCCTCCGGCTACTCCTTTA | |
| | CCGACTACTACATGAACTGGCTGAGGCAA | |
| | GCTCCCGGTCAAGGTCTGGAGTGGATCGG | |
| | CGATATCAACCCCAAGGACGGCGACTCCG | |
| | GCTACAGCCATAAGTTCAAGGGTCGTGCC | |
| | ACTTTAACCGTGGACAAGTCCACCAGCAC | |
| | CGTGTACATGGAGCTGAGGTCTTTAAGGT | |
| | CCGAGGACACCGCCGTGTACTACTGCGCC | |
| | TCCGGCTTCACCACAGTGGTGGCTCGTGGC | |
| | GACTATTGGGGCCAAGGTACCACCGTGAC | |
| | CGTGAGCTCC | |
| 59 | DVVMTQSPLSLPVTLGQPASISCKSSQSLLDS | Hu22F1-La VL |
| | DGKTYLNWLQQRPGQSPRRLIYLVSKLDSG | |
| | VPDRFSGSGSGTDFTLKISRVEAEDVGVYYC | |
| | WQGTHFPYTFGGGTKVEIK | |
| 60 | GATGTGGTGATGACACAGTCTCCTCTGTCT | Hu22F1-La VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTACCTGAATT | |
| | GGCTGCAGCAGAGACCAGGACAGTCTCCT | |
| | AGAAGACTGATCTACCTGGTGTCCAAGCT | |
| | GGATTCTGGAGTGCCAGATAGATTTTCCG | |
| | GCTCCGGCTCTGGCACAGATTTCACCCTGA | |
| | AGATCTCTAGAGTGGAGGCAGAAGACGTG | |
| | GGAGTGTACTATTGTTGGCAGGGAACCCA | |
| | CTTCCCTTACACATTTGGAGGAGGCACAA | |
| | AGGTGGAGATCAAG | |
| 61 | DVVMTQSPLSLPVTLGQPASISCKSSQSLLDS | Hu22F1-Lb VL |
| | DGKTYLNWLQQRPGQSPRRLIYLVSKLDSG | |
| | FPDRFSGSGSGTDFTLKISRVEAEDVGVYYC | |
| | WQGTHFPYTFGGGTKVEIK | |
| 62 | GATGTGGTGATGACACAGTCTCCTCTGTCT | Hu22F1-Lb VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTACCTGAATT | |
| | GGCTGCAGCAGAGACCAGGACAGTCTCCT | |
| | AGAAGACTGATCTACCTGGTGTCCAAGCT | |
| | GGATTCTGGATTCCCAGATAGATTTTCCGG | |
| | CTCCGGCTCTGGCACAGATTTCACCCTGAA | |
| | GATCTCTAGAGTGGAGGCAGAAGACGTGG | |
| | GAGTGTACTATTGTTGGCAGGGAACCCAC | |
| | TTCCCTTACACATTTGGAGGAGGCACAAA | |
| | GGTGGAGATCAAG | |
| 63 | NSFYIPRHIRKEEGSFQSCSF | BMP-binding loop |
| 64 | FSYSVPNTFPQSTESLVHCDS | the 63-83 amino |
| | | acids of DAN |
| 65 | MGWSCIILFLVATGVHS | signal peptide |
| 66 | MSRTAYTVGALLLLLGTLLPAAEGKKKGSQ | human gremlin 1 |
| | GAIPPPDKAQHNDSEQTQSPQQPGSRNRGRG | |
| | QGRGTAMPGEEVLESSQEALHVTERKYLKR | |
| | DWCKTQPLKQTIHEEGCNSRTIINRFCYGQC | |
| | NSFYIPRHIRKEEGSFQSCSFCKPKKFTTMMV | |
| | TLNCPELQPPTKKKRVTRVKQCRCISIDLD | |
| 67 | MNRTAYTVGALLLLLGTLLPTAEGKKKGSQ | mouse gremlin 1 |
| | GAIPPPDKAQHNDSEQTQSPPQPGSRTRGRG | |
| | QGRGTAMPGEEVLESSQEALHVTERKYLKR | |
| | DWCKTQPLKQTIHEEGCNSRTIINRFCYGQC | |
| | NSFYIPRHIRKEEGSFQSCSFCKPKKFTTMMV | |
| | TLNCPELQPPTKKKRVTRVKQCRCISIDLD | |
| 68 | MSRTAYTVGALLLLLGTLLPAAEGKKKGSQ | chimeric hGREM1 |
| | GAIPPPDKAQHNDSEQTQSPQQPGSRNRGRG | |
| | QGRGTAMPGEEVLESSQEALHVTERKYLKR | |
| | DWCKTQPLKQTIHEEGCNSRTIINRFCYGQC | |
| | FSYSVPNTFPQSTESLVHCDSCKPKKFTTMM | |
| | VTLNCPELQPPTKKKRVTRVKQCRCISIDLD | |
| 69 | KKKGSQGAIPPPDKAQHNDSEQTQSPQQPGS | Human Gremlin 1 |
| | RNRGRGQGRGTAMPGEEVLESSQEALHVTE | sequence without |
| | RKYLKRDWCKTQPLKQTIHEEGCNSRTIINR | signal peptide |
| | FCYGQCNSFYIPRHIRKEEGSFQSCSFCKPKK | |
| | FTTMMVTLNCPELQPPTKKKRVTRVKQCRC | |
| | ISIDLD | |
| 70 | KKKGSQGAIPPPDKAQHNDSEQTQSPPQPGS | Mouse Gremlin 1 |
| | RTRGRGQGRGTAMPGEEVLESSQEALHVTE | sequence without |
| | RKYLKRDWCKTQPLKQTIHEEGCNSRTIINR | signal peptide |
| | FCYGQCNSFYIPRHIRKEEGSFQSCSFCKPKK | |
| | FTTMMVTLNCPELQPPTKKKRVTRVKQCRC | |
| | ISIDLD | |
| 71 | MSRTAYTVGALLLLLGTLLPAAEG | Human Gremlin 1 |
| | | signal peptide |
| 72 | MNRTAYTVGALLLLLGTLLPTAEG | Mouse Gremlin 1 |
| | | signal peptide |
| 73 | MEEPQSDPSVEPPLSQETFSDLWKLLPENNV | p53 protein |
| | LSPLPSQAMDDLMLSPDDIEQWFTEDPGP | PTEN protein |
| | DEAPRMPEAAPPVAPAPAAPTPAAPAPAPS | |
| | WPLSSSVPSQKTYQGSYGFRLGFLHSGTAK | |
| | SVTCTYSPALNKMFCQLAKTCPVQLWVDST | |
| | PPPGTRVRAMAIYKQSQHMTEVVRRCPHHE | |
| | RCSDSDGLAPPQHLIRVEGNLRVEYLDDRNT | |
| | FRHSVVVPYEPPEVGSDCTTIHYNYMCNS | |
| | SCMGGMNRRPILTIITLEDSSGNLLGRNSFEV | |
| | RVCACPGRDRRTEEENLRKKGEPHHELP | |
| | PGSTKRALPNNTSSSPQPKKKPLDGEYFTLQI | |
| | RGRERFEMFRELNEALELKDAQAGKEPG | |
| | GSRAHSSHLKSKKGQSTSRHKKLMFKTEGP | |
| | DSD | |
| 74 | MTAIIKEIVS RNKRRYQEDG FDLDLTYIYP | isoform 1 |
| | NIIAMGFPAE RLEGVYRNNI DDVVRFLDSK | |
| | HKNHYKIYNL CAERHYDTAK NCRVAQYPF | |
| | EDHNPPQLEL IKPFCEDLDQ WLSEDDNHVA | |
| | AIHCKAGKGR TGVMICAYLL RGKFLKAQE | |
| | ALDFYGEVRT RDKKGVTIPS RRYVYYYSY | |
| | LLKNHLDYRP VALLFHKMMF ETIPMFSGGT | |
| | CNPQFVVCQL KVKIYSSNSG PTRREDKFMY | |
| | FEFPQPLPVC GDIKVEFFHK NKMLKKDKM | |
| | FHFWVNTFFI PGPEETSEKV ENGSLCDQEI | |
| | DSICSIERAD NDKEYLVLTL TKNDLDKANK | |
| | DKANRYFSPN FKVKLYFTKT VEEPSNPEAS | |
| | SSTSVTPDVS DNEPDHYRYS DTTDSDPENE | |
| | PFDEDQHTQI TKV | |
| 75 | SWKCLLFWAVLVTATLCTARPSPTLPEQAQ | human FGFR1 |
| | PWGAPVEVESFLVHPGDLLQLRCRLRDDVQ | |
| | SINWLRDGVQLAESNRTRITGEEVEVQDSVP | |
| | ADSGLYACVTSSPSGSDTTYFSVNVSDALPS | |
| | SEDDDDDDDSSSEEKETDNTKPNRMPVAPY | |
| | WTSPEKMEKKLHAVPAAKTVKFKCPSSGTP | |
| | NPTLRWLKNGKEFKPDHRIGGYKVRYATWS | |
| | IIMDSVVPSDKGNYTCIVENEYGSINHTYQL | |
| | DVVERSPHRPILQAGLPANKTVALGSNVEF | |
| | MCKVYSDPQPHIQWLKHIEVNGSKIGPDNLP | |
| | YVQILKTAGVNTTDKEMEVLHLRNVSFEDA | |
| | GEYTCLAGNSIGLSHHSAWLTVLEALEERPA | |
| | VMTSPLYLEIIIYCTGAFLISCMVGSVIVYKM | |
| | KSGTKKSDFHSQMAVHKLAKSIPLRRQVTV | |
| | SADSSASMNSGVLLVRPSRLSSSGTPMLAGV | |
| | SEYELPEDPRWELPRDRLVLGKPLGEGCFGQ | |
| | VVLAEAIGLDKDKPNRVTKVAVKMLKSDA | |
| | TEKDLSDLISEMEMMKMIGKHKNIINLLGAC | |
| | TQDGPLYVIVEYASKGNLREYLQARRPPGLE | |
| | YCYNPSHNPEEQLSSKDLVSCAYQVARGME | |
| | YLASKKCIHRDLAARNVLVTEDNVMKIADF | |
| | GLARDIHHIDYYKKTTNGRLPVKWMAPEAL | |
| | FDRIYTHQSDVWSFGVLLWEIFTLGGSPYPG | |
| | VPVEELFKLLKEGHRMDKPSNCTNELYMM | |
| | MRDCWHAVPSQRPTFKQLVEDLDRIVALTS | |
| | NQEYLDLSMPLDQYSPSFPDTRSSTCSSGED | |
| | SVFSHEPLPEEPCLPRHPAQLANGGLKRR | |

1. A method of treating a GREM1-expressing cancer in a subject in need thereof, comprising administering to the subject a therapeutically effective amount of GREM1 antagonist, wherein the cancer is characterized in reduced androgen receptor (AR) signaling.

2. The method of claim 1, wherein the cancer is a) an AR-expressing cancer or is an AR negative cancer, and/or b) selected from the group consisting of prostate cancer, breast cancer, lung cancer, head and neck cancer, testis cancer, endometrial cancer, ovarian cancer, and skin cancer, and/or c) metastatic, optionally, the cancer is metastatic prostate cancer, further optionally, the cancer is lung metastasis of prostate cancer, and/or d) characterized in GREM1 overexpression.

3. (canceled)

4. The method of claim 1, wherein the subject is receiving or has received an androgen deprivation therapy, or is resistant to an androgen deprivation therapy.

5-6. (canceled)

7. A method of increasing sensitivity of an AR-expressing cancer to an androgen deprivation therapy in a subject, comprising administering to the subject a therapeutically effective amount of GREM1 antagonist.

8. A method of treating a GREM1-related disease or condition characterized in deficiency in PTEN and/or p53 in a subject in need thereof, or inhibiting FGFR1 activation in a subject in need thereof, or inhibiting MAPK signaling in a subject in need thereof, comprising administering to the subject a therapeutically effective amount of GREM1 antagonist.

9. The method of claim 8, wherein the deficiency in PTEN and/or p53 is characterized in absence of functional PTEN and/or p53, or in absence of PTEN and/or p53 expression.

10. The method of claim 9, wherein the deficiency in PTEN and/or p53 is characterized in the presence of inactivating mutation in PTEN and/or p53.

11. (canceled)

12. The method of claim 8, wherein the GREM1 related disease or condition is characterized in GREM1 expression or overexpression.

13. The method of claim 8, wherein the GREM1-related disease or condition is selected from the group consisting of cancer, fibrotic disease, angiogenesis, glaucoma or retinal disease, kidney disease, pulmonary arterial hypertension, and osteoarthritis (OA).

14. (canceled)

15. The method of claim 1, wherein the cancer is prostate cancer, breast cancer, glioma, liposarcoma, hepatocellular carcinoma, lung cancer, cervical cancer, endometrial carcinoma, uterine leiomyosarcoma, squamous cell carcinoma of the head and neck, thyroid cancer, liver cancer, pancreatic cancer, bladder cancer, colon cancer, esophageal cancer, bile duct cancer, osteosarcoma, glioblastoma, ovarian cancer, gastric cancer, triple negative breast cancer (TNBC), small cell lung cancer or melanoma.

16. (canceled)

17. The method of claim 2, wherein the prostate cancer is:
a) negative in androgen receptor (AR) expression,
b) negative in both androgen receptor (AR) expression and neuroendocrine (NE) differentiation;
c) resistant to an androgen deprivation therapy, optionally castration-resistant,
d) showing a level of Prostate Specific Antigen (PSA) lower than a reference level, or
e) any combinations of a) to d).

18. (canceled)

19. The method of claim 2, wherein the breast cancer is triple negative breast cancer.

20. The method of claim 13, wherein the fibrotic disease is lung fibrosis, skin fibrosis, diabetic nephropathy, or ischaemic renal injury.

21-22. (canceled)

23. The method of claim 1, wherein the GREM1 antagonist comprises an anti-GREM1 antibody or antigen-binding fragment thereof, an inhibitory GREM1 mimetic peptide, an inhibitory nucleic acid targeting GREM1 RNA or DNA, a polynucleotide encoding the inhibitory nucleic acid, a compound inhibiting interaction between gremlin and BMP, or a compound inhibiting the GREM1 activity.

24. (canceled)

25. The method of claim 1, wherein the GREM1 antagonist comprises a GREM1-FGFR1 axis inhibitor.

26-27. (canceled)

28. The method of claim 25, wherein the GREM1-FGFR1 axis inhibitor comprises an FGFR1-binding inhibitor.

29-30. (canceled)

31. The method of claim 25, wherein the GREM1 antagonist or GREM1-FGFR1 axis inhibitor comprises an antibody against hGREM1 or an antigen-binding fragment thereof.

32-36. (canceled)

37. The method of claim 31, wherein the antibody against hGREM1 or antigen-binding fragment thereof comprises:
a) the heavy chain variable region comprises a HCDR1 comprising the sequence of SEQ ID NO: 1, a HCDR2 comprising the sequence of SEQ ID NO: 2, and a HCDR3 comprising the sequence of SEQ ID NO: 3; and the light chain variable region comprises a LCDR1 comprising the sequence of SEQ ID NO: 4, a LCDR2 comprising the sequence of SEQ ID NO: 5, and a LCDR3 comprising the sequence of SEQ ID NO: 6;
b) the heavy chain variable region comprises a HCDR1 comprising the sequence of SEQ ID NO: 11, a HCDR2 comprising the sequence of SEQ ID NO: 12, and a HCDR3 comprising the sequence of SEQ ID NO: 13; and the light chain variable region comprises a LCDR1 comprising the sequence of SEQ ID NO: 14, a LCDR2 comprising the sequence of SEQ ID NO: 15, and a LCDR3 comprising the sequence of SEQ ID NO: 16;
c) the heavy chain variable region comprises a HCDR1 comprising the sequence of SEQ ID NO: 21, a HCDR2 comprising the sequence of SEQ ID NO: 22, and a HCDR3 comprising the sequence of SEQ ID NO: 23; and the light chain variable region comprises a LCDR1 comprising the sequence of SEQ ID NO: 24, a LCDR2 comprising the sequence of SEQ ID NO: 25, and a LCDR3 comprising the sequence of SEQ ID NO: 26; or
d) the heavy chain variable region comprises a HCDR1 comprising the sequence of SEQ ID NO: 31, a HCDR2 comprising the sequence of SEQ ID NO: 32, and a HCDR3 comprising the sequence of SEQ ID NO: 33; and the light chain variable region comprises a LCDR1 comprising the sequence of SEQ ID NO: 34, a LCDR2 comprising the sequence of SEQ ID NO: 35, and a LCDR3 comprising the sequence of SEQ ID NO: 36.

38-39. (canceled)

40. The method of claim 31, wherein the antibody against hGREM1 or antigen-binding fragment thereof comprises:
a) a heavy chain variable region comprising the sequence of SEQ ID NO: 7 and a light chain variable region comprising the sequence of SEQ ID NO: 8; or
b) a heavy chain variable region comprising a sequence of SEQ ID NO: 17 and a light chain variable region comprising a sequence of SEQ ID NO: 18; or
c) a heavy chain variable region comprising a sequence of SEQ ID NO: 27 and a light chain variable region comprising a sequence of SEQ ID NO: 28; or
d) a heavy chain variable region comprising a sequence of SEQ ID NO: 37 and a light chain variable region comprising a sequence of SEQ ID NO: 38; or
e) a heavy chain variable region comprising a sequence selected from the group consisting of SEQ ID NO: 41, SEQ ID NO: 43 and SEQ ID NO: 45, and a light chain variable region comprising a sequence selected from the group consisting of SEQ ID NO: 47 and SEQ ID NO: 49; or
f) a pair of heavy chain variable region and light chain variable region sequences selected from the group consisting of: SEQ ID NOs: 41/47, 41/49, 43/47, 43/49, 45/47, and 45/49; or
g) a heavy chain variable region comprising a sequence selected from the group consisting of SEQ ID NO: 51, SEQ ID NO: 53, SEQ ID NO: 55 and SEQ ID NO: 57, and a light chain variable region comprising a sequence selected from the group consisting of SEQ ID NO: 59 and SEQ ID NO: 61; or
h) a pair of heavy chain variable region and light chain variable region sequences selected from the group consisting of: SEQ ID NOs: 51/59, 51/61, 53/59, 53/61, 55/59, 55/61, 57/59, and 57/61.

41-42. (canceled)

43. The method of claim 31, wherein the antibody against hGREM1 or antigen-binding fragment thereof further comprises an immunoglobulin constant region, optionally a constant region of human IgG, or optionally a constant region of human IgG1, IgG2, IgG3, or IgG4.

44-46. (canceled)

47. The method of claim 31, wherein the antibody against hGREM1 or antigen-binding fragment thereof is humanized and/or bispecific.

48. The method of claim 31, wherein the antibody against hGREM1 or antigen-binding fragment thereof is capable of specifically binding to a first and a second epitope of gremlin, or capable of specifically binding to both hGREM1 and a second antigen.

49. The method of claim 48, wherein the second antigen comprises an immune related target or a tumor antigen, wherein the immune related target is selected from the group consisting of: PD-1, PD-L1, PD-L2, CTLA-4, TIM-3, LAG3, A2AR, CD160, 2B4, TGFβ, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 or CD83; wherein the tumor antigen is selected from the group consisting of: prostate specific antigen (PSA), CA-125, gangliosides G(D2), G(M2) and G(D3), CD20, CD52, CD33, Ep-CAM, CEA, bombesin-like peptides, HER2/neu, epidermal growth factor receptor (EGFR), erB2, erbB3/HER3, erbB4, CD44v6, Ki-67, cancer-associated mucin, VEGF, VEGFRs (e.g., VEGFR-1, VEGFR-2, VEGFR-3), estrogen receptor, Lewis-Y antigen, TGFβ1, IGF-1 receptor, EGFα, c-Kit receptor, transferrin receptor, Claudin 18.2, GPC-3, Nectin-4, ROR1, methothelin, PCMA, MAGE-1, MAGE-3, BAGE, GAGE-1, GAGE-2, pl5, BCR-ABL, E2APRL, H4-RET, IGH-IGK, MYL-RAR, IL-2R, CO17-1A, TROP2, and LIV-1.

50-55. (canceled)

56. The method of claim 1, further comprising administering a therapeutically-effective amount of a second therapeutic agent, wherein the second therapeutic agent comprises anti-cancer therapy, or wherein the anti-cancer therapy is selected from a chemotherapeutic agent, radiation therapy, an immunotherapy agent, anti-angiogenesis agent (e.g., antagonist of a VEGFR such as VEGFR-1, VEGFR-2, and VEGFR-3), a targeted therapy agent, a cellular therapy agent, a gene therapy agent, a hormonal therapy agent, cytokines, palliative care, surgery for the treatment of cancer (e.g., tumorectomy), one or more anti-emetics, treatments for complications arising from chemotherapy, or a diet supplement for cancer patients (e.g. indole-3-carbinol).

57. (canceled)

58. The method of claim 56, wherein the anti-cancer therapy comprises an anti-prostate cancer drug selected from the group consisting of: androgen deprivation therapy; androgen axis inhibitor; and androgen synthesis inhibitor; a PARP inhibitor; Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Histrelin (Vantas), Jevtana (Cabazitaxel), Leuprolide Acetate, Lupron (Leuprolide Acetate), Lupron Depot (Leuprolide Acetate), Lynparza (Olaparib), Ketoconazole (Nizoral), Mitoxantrone Hydrochloride, Nilandron (Nilutamide), Nilutamide, Nubeqa (Darolutamide), Olaparib, Provenge (Sipuleucel-T), Radium 223 Dichloride, Relugolix (Orgovyx), Rubraca (Rucaparib Camsylate), Rucaparib Camsylate, Sipuleucel-T, Taxotere (Docetaxel), Triptorelin (Trelstar), Xofigo (Radium 223 Dichloride), Xtandi (Enzalutamide), Zoladex (Goserelin Acetate) and Zytiga (Abiraterone Acetate) or any combination thereof.

59-60. (canceled)

61. The method of claim 58, wherein the androgen axis inhibitor is degarelix, bicalutamide, flutamide, nilutamide, apalutamide, darolutamide, enzalutamide, or abiraterone.

62-77. (canceled)


SEQUENCE LISTING (75 sequences)

SEQ ID NO: 1 - protein, 5 aa, Artificial Sequence
  Synthetic
TYGMA

SEQ ID NO: 2 - protein, 17 aa, Artificial Sequence
  Synthetic
WINTLSGEPTYADDFKG

SEQ ID NO: 3 - protein, 5 aa, Artificial Sequence
  Synthetic
EPMDY

SEQ ID NO: 4 - protein, 16 aa, Artificial Sequence
  Synthetic
KSSQSLLDSDGKTYLS

SEQ ID NO: 5 - protein, 7 aa, Artificial Sequence
  Synthetic
LVSKLDS

SEQ ID NO: 6 - protein, 9 aa, Artificial Sequence
  Synthetic
WQGAHFPLT

SEQ ID NO: 7 - protein, 114 aa, Artificial Sequence
  Synthetic
QIQLVQSGPELKKPGETVKISCKTSGSTFTTYGMAWMKQAPGKGLTWMGWINTLSGEPTYADDFKGRFAFSLKTSANTAYLQINNLKNEDAATYFCAREPMDYWGQGTSVIVSS

SEQ ID NO: 8 - protein, 112 aa, Artificial Sequence
  Synthetic
DVVMTQTPLTLSITIGQPASISCKSSQSLLDSDGKTYLSWLLQRPDQSPKRLISLVSKLDSGVPDRITGSGSGTDFTLKISRVEAEDLGIYYCWQGAHFPLTFGAGTKLELK

SEQ ID NO: 9 - DNA, 342 bp, Artificial Sequence
  Synthetic
cagatccagttggtacagtctggacctgaactgaagaagcctggagagacagtcaagatctcctgcaagacttctggatctacgttcacaacctatggaatggcctggatgaagcaggctccaggaaagggtttaacgtggatgggctggataaacaccctctctggagagccaacatatgctgatgacttcaagggacggtttgccttctctttgaaaacctctgccaacactgcctatttgcagatcaacaacctcaaaaatgaggacgcggctacatatttctgtgcacgagaaccaatggactactggggtcaaggaacctcagtcatcgtctcctca

SEQ ID NO: 10 - DNA, 336 bp, Artificial Sequence
  Synthetic
gatgttgtgatgacccagactccactcactttgtcgattaccattggacaaccagcctccatctcttgcaaatcaagtcagagcctcttagatagtgatggaaagacatatttgagttggttgttacagaggccagaccagtctccaaagcgcctaatctctctggtgtccaaactggactctggagtccctgacaggatcactggcagtggatcagggacagatttcacactgaaaatcagcagagtggaggctgaagatttgggcatctattattgctggcaaggtgcacattttccgctcacgttcggtgctgggaccaagctggagctgaaa

SEQ ID NO: 11 - protein, 5 aa, Artificial Sequence
  Synthetic
DYYMN

SEQ ID NO: 12 - protein, 17 aa, Artificial Sequence
  Synthetic
DINPKDGDSGYSHKFKG

SEQ ID NO: 13 - protein, 11 aa, Artificial Sequence
  Synthetic
GFTTVVARGDY

SEQ ID NO: 14 - protein, 16 aa, Artificial Sequence
  Synthetic
KSSQSLLDSDGKTYLN

SEQ ID NO: 15 - protein, 7 aa, Artificial Sequence
  Synthetic
LVSKLDS

SEQ ID NO: 16 - protein, 9 aa, Artificial Sequence
  Synthetic
WQGTHFPYT

SEQ ID NO: 17 - protein, 120 aa, Artificial Sequence
  Synthetic
EAQLQQSGPELVKPGASVKISCKASGYSFTDYYMNWLKQSHGKSLEWIGDINPKDGDSGYSHKFKGKATLTVDKSSSTAYMELRSLTSEDSAVYYCASGFTTVVARGDYWGQGTTLTVSS

SEQ ID NO: 18 - protein, 112 aa, Artificial Sequence
  Synthetic
DVVMTQTPLTLSVTIGQPASISCKSSQSLLDSDGKTYLNWLLQRPGQSPKRLIYLVSKLDSGFPDRFTGSGSGTDFTLKISRVEAEDLGVYYCWQGTHFPYTFGGGTKLEIK

SEQ ID NO: 19 - DNA, 360 bp, Artificial Sequence
  Synthetic
gaggcccagctgcaacaatctggacctgaactggtgaagcctggggcttcagtgaagatatcctgtaaggcttctggatactcgttcactgactactacatgaactggctgaagcagagccatggaaagagccttgagtggattggagatattaatcctaaagatggtgatagtggttacagccataagttcaagggcaaggccacattgactgtagacaagtcctccagcacagcctacatggagctccgcagcctgacatctgaggactctgcagtctattactgtgcaagcggatttaccacggtagtagctaggggggactactggggccaaggcaccactctcacagtctcctca

SEQ ID NO: 20 - DNA, 336 bp, Artificial Sequence
  Synthetic
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatttggtgtctaaactggactctggattccctgacaggttcactggcagtggatcagggacagatttcacactgaaaatcagcagagtggaggctgaggatttgggagtttattattgctggcaaggtacacattttccgtacacgttcggaggggggaccaagctggaaataaaa

SEQ ID NO: 21 - protein, 5 aa, Artificial Sequence
  Synthetic
DDYMH

SEQ ID NO: 22 - protein, 17 aa, Artificial Sequence
  Synthetic
WIDPENGDTEYASKFQG

SEQ ID NO: 23 - protein, 9 aa, Artificial Sequence
  Synthetic
WATVPDFDY

SEQ ID NO: 24 - protein, 17 aa, Artificial Sequence
  Synthetic
KSSQSLLNRSNQKNYLA

SEQ ID NO: 25 - protein, 7 aa, Artificial Sequence
  Synthetic
FTSTRES

SEQ ID NO: 26 - protein, 9 aa, Artificial Sequence
  Synthetic
QQHYSTPFT

SEQ ID NO: 27 - protein, 118 aa, Artificial Sequence
  Synthetic
EVQLQQSGAELVRPGASVKLSCTASGFNIKDDYMHWVKRRPEQGLEWIGWIDPENGDTEYASKFQGKATITADTSSNTAYLQLSSLTSEDTAVYYCTTWATVPDFDYWGQGTTLTVSS

SEQ ID NO: 28 - protein, 113 aa, Artificial Sequence
  Synthetic
DIVMTQSPSSLAMSVGQKVTMSCKSSQSLLNRSNQKNYLAWYQQKPGQSPKLLVHFTSTRESGVPDRFIGSGSGTDFTLTISNLQAEDLADYFCQQHYSTPFTFGSGTKLEIK

SEQ ID NO: 29 - DNA, 354 bp, Artificial Sequence
  Synthetic
gaggtgcagctgcaacagtccggcgctgaactggtgaggcctggagcctccgtgaagctgtcctgcaccgccagcggcttcaacatcaaggacgactacatgcactgggtgaagaggaggcctgagcagggcctggagtggatcggctggatcgaccccgagaacggcgacaccgagtacgcctccaagttccagggcaaggccaccatcaccgccgacacctcctccaacaccgcctacctgcagctgagctccctgacctccgaggacaccgccgtgtactattgcaccacctgggccaccgtgcccgacttcgactactggggacagggcaccaccctgaccgtgtccagc

SEQ ID NO: 30 - DNA, 339 bp, Artificial Sequence
  Synthetic
gatatcgtgatgacccagtctccttcctctctggctatgtcagtgggacagaaagtgaccatgtcttgcaagtcctctcagtctctgctgaacaggtccaaccagaagaactacctggcttggtaccagcagaaaccaggacagtctcctaagctgctggtgcattttacctctaccagggaatccggagtgccagatagatttatcggctctggctccggcacagattttacactgaccatctccaatctgcaggcagaagatctggctgactacttttgccagcagcactactccaccccttttacctttggctccggcaccaagctggagatcaag

SEQ ID NO: 31 - protein, 5 aa, Artificial Sequence
  Synthetic
DFYMN

SEQ ID NO: 32 - protein, 17 aa, Artificial Sequence
  Synthetic
DINPNNGGTSYNQKFKG

SEQ ID NO: 33 - protein, 12 aa, Artificial Sequence
  Synthetic
DPIYYDYDEVAY

SEQ ID NO: 34 - protein, 16 aa, Artificial Sequence
  Synthetic
RSSQSLVHSNGNTYLH

SEQ ID NO: 35 - protein, 7 aa, Artificial Sequence
  Synthetic
KVSNRFS

SEQ ID NO: 36 - protein, 9 aa, Artificial Sequence
  Synthetic
SQSTHVPLT

SEQ ID NO: 37 - protein, 121 aa, Artificial Sequence
  Synthetic
EVQLQQSGPELVKPGASVKISCKASGYTFTDFYMNWVKQSHGKSLEWIGDINPNNGGTSYNQKFKGKATLTVDKSSSTAYMELRSLTSEDSAVYYCARDPIYYDYDEVAYWGQGTLVTVSA

SEQ ID NO: 38 - protein, 112 aa, Artificial Sequence
  Synthetic
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 39 - DNA, 363 bp, Artificial Sequence
  Synthetic
gaggtgcagctgcagcagtccggccctgagctggtgaagcctggagcctccgtgaagatctcctgtaaggcctccggctacaccttcaccgacttctacatgaactgggtgaagcagtcccacggcaagtccctggagtggatcggcgacatcaatcccaacaacggcggcacctcctacaaccagaagttcaagggcaaggccaccctgacagtggacaagtcctccagcaccgcctacatggagctgaggtccctgacctccgaggactccgccgtgtactactgcgccagggaccccatctactacgactacgacgaggtggcctactggggccagggaaccctggtgacagtgtccgcc

SEQ ID NO: 40 - DNA, 336 bp, Artificial Sequence
  Synthetic
gatgtggtgatgacacagacacctctgtctctgccagtgtctctcggagatcaggcttctatctcttgcagatcctctcagtctctggtgcattccaacggaaacacctacctgcattggtacctgcagaaaccaggacagtctcctaagctgctgatctacaaggtgtccaacaggttctccggagtgccagatagattttccggatctggatctggcaccgattttaccctgaagatctctagagtggaagcagaggatctgggagtgtacttttgtagccagtctacccacgtgcctctgacatttggagcaggaacaaagctggagctgaag

SEQ ID NO: 41 - protein, 114 aa, Artificial Sequence
  Synthetic
QVQLVQSGSELKKPGASVKVSCKASGYTFTTYGMAWMRQAPGQGLEWMGWINTLSGEPTYADDFKGRFVFSLDTSVSTAYLQISSLKAEDTAVYYCAREPMDYWGQGTMVTVSS

SEQ ID NO: 42 - DNA, 342 bp, Artificial Sequence
  Synthetic
caggtgcagctggtgcagtccggctccgagctgaagaagcctggcgcctccgtgaaggtgtcctgcaaggcctccggctacaccttcaccacctacggcatggcctggatgaggcaggctcctggccagggactggagtggatgggctggatcaacaccctgtccggcgaacccacctacgccgacgacttcaagggcaggttcgtgttctccctggacaccagcgtgtccaccgcctacctgcagatctcctccctgaaggccgaggacaccgccgtgtactactgcgccagggagcccatggactactggggccagggcaccatggtgaccgtgtcctcc

SEQ ID NO: 43 - protein, 114 aa, Artificial Sequence
  Synthetic
QIQLVQSGSELKKPGASVKVSCKASGYTFTTYGMAWMRQAPGQGLEWMGWINTLSGEPTYADDFKGRFAFSLDTSVSTAYLQISSLKAEDTAVYYCAREPMDYWGQGTMVTVSS

SEQ ID NO: 44 - DNA, 342 bp, Artificial Sequence
  Synthetic
cagatccagctggtgcagagcggcagcgagctgaagaagcccggcgctagcgtgaaggtgtcctgcaaggccagcggctacaccttcaccacctacggcatggcctggatgaggcaggctcctggacagggcctggagtggatgggctggatcaacaccctgtccggcgagcctacctacgccgacgacttcaagggcaggttcgccttctccctggacacctccgtgagcaccgcctacctgcagatctccagcctgaaggccgaggacaccgccgtgtactactgcgccagggagcctatggactactggggccagggcaccatggtgaccgtgtccagc

SEQ ID NO: 45 - protein, 114 aa, Artificial Sequence
  Synthetic
QIQLVQSGSELKKPGASVKVSCKASGSTFTTYGMAWMKQAPGQGLTWMGWINTLSGEPTYADDFKGRFAFSLDTSVSTAYLQISSLKAEDTAVYYCAREPMDYWGQGTMVTVSS

SEQ ID NO: 46 - DNA, 342 bp, Artificial Sequence
  Synthetic
cagatccagctggtgcagtccggcagcgagctcaagaagcccggagccagcgtgaaggtgtcctgcaaggccagcggctccaccttcaccacatacggcatggcctggatgaagcaggctcctggccagggcctgacctggatgggatggatcaacaccctgtccggcgagcctacctacgccgatgacttcaagggcaggttcgccttctccctggacacctccgtgtccaccgcttacctgcagatctcctccctgaaggccgaggacaccgccgtgtactactgcgccagggagcccatggactactggggccagggcaccatggtgaccgtgtcctcc

SEQ ID NO: 47 - protein, 112 aa, Artificial Sequence
  Synthetic
DVVMTQSPLSLPVTLGQPASISCKSSQSLLDSDGKTYLSWLQQRPGQSPRRLIYLVSKLDSGVPDRFSGSGSGTDFTLKISRVEAEDVGVYYCWQGAHFPLTFGQGTKLEIK

SEQ ID NO: 48 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctatctgtcttggctgcagcagagaccaggacagtctcctagaagactgatctacctggtgtccaagctggattctggagtgccagatagattttccggctccggctctggcacagatttcaccctgaagatctctagagtggaggcagaagacgtgggagtgtactattgttggcagggagctcacttccctctgacatttggacagggaacaaagctggagatcaag

SEQ ID NO: 49 - protein, 112 aa, Artificial Sequence
  Synthetic
DVVMTQSPLSLPVTLGQPASISCKSSQSLLDSDGKTYLSWLQQRPGQSPRRLISLVSKLDSGVPDRFSGSGSGTDFTLKISRVEAEDVGVYYCWQGAHFPLTFGQGTKLEIK

SEQ ID NO: 50 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctatctgtcttggctgcagcagagaccaggacagtctcctagaagactgatctccctggtgtctaagctggattccggagtgccagatagattttccggatctggatctggcaccgattttaccctgaagatctctagagtggaggcagaagacgtgggagtgtactattgttggcagggagctcacttccctctgacatttggacagggaacaaagctggagatcaag

SEQ ID NO: 51 - protein, 120 aa, Artificial Sequence
  Synthetic
QVQLVQSGAEVKKPGASVKVSCKASGYSFTDYYMNWVRQAPGQGLEWMGDINPKDGDSGYSHKFKGRVTMTRDTSTSTVYMELSSLRSEDTAVYYCASGFTTVVARGDYWGQGTTVTVSS

SEQ ID NO: 52 - DNA, 360 bp, Artificial Sequence
  Synthetic
caagttcagctggtgcagtccggagccgaggtgaagaagcccggcgcttccgtgaaggtgtcttgtaaggcctccggctactccttcaccgattactacatgaactgggtgaggcaagctcccggtcaaggtctggagtggatgggcgacatcaaccccaaggacggcgactccggctattcccacaagttcaagggtcgtgtgaccatgaccagggacacgtccaccagcaccgtgtacatggagctgtcctctttaaggtccgaggacaccgccgtgtactactgcgccagcggattcaccaccgtggtggctaggggcgactattggggccaaggtaccaccgtgacagtgtccagc

SEQ ID NO: 53 - protein, 120 aa, Artificial Sequence
  Synthetic
QVQLVQSGAEVKKPGASVKVSCKASGYSFTDYYMNWVRQAPGQGLEWMGDINPKDGDSGYSHKFKGRVTMTVDKSTSTVYMELSSLRSEDTAVYYCASGFTTVVARGDYWGQGTTVTVSS

SEQ ID NO: 54 - DNA, 360 bp, Artificial Sequence
  Synthetic
caagttcagctggtgcagtccggagccgaggtgaagaagcccggcgcttccgtgaaggtgtcttgtaaggcctccggctactccttcaccgattactacatgaactgggtgaggcaagctcccggtcaaggtctggagtggatgggcgacatcaaccccaaggacggcgactccggctattcccacaagttcaagggtcgtgtgaccatgaccgtggacaagtccaccagcaccgtgtacatggagctgtcctctttaaggtccgaggacaccgccgtgtactactgcgccagcggattcaccaccgtggtggctaggggcgactattggggccaaggtaccaccgtgacagtgtccagc

SEQ ID NO: 55 - protein, 120 aa, Artificial Sequence
  Synthetic
QAQLVQSGAEVKKPGASVKVSCKASGYSFTDYYMNWVRQAPGQGLEWMGDINPKDGDSGYSHKFKGRVTLTVDKSTSTVYMELRSLRSEDTAVYYCASGFTTVVARGDYWGQGTTVTVSS

SEQ ID NO: 56 - DNA, 360 bp, Artificial Sequence
  Synthetic
caagctcagctggtgcagtccggcgctgaggtgaaaaagcccggcgccagcgtgaaggtgtcttgtaaggcctccggctactccttcaccgactactacatgaactgggtgaggcaagctcccggtcaaggtctggagtggatgggcgacatcaaccccaaggacggcgacagcggctactcccacaagttcaagggtcgtgtgactttaaccgtggacaagtccacctccaccgtctacatggagctgaggtctttaaggtccgaggataccgccgtgtactactgcgctagcggcttcaccaccgtggtggctcgtggcgattactggggacaaggtaccaccgtgaccgtgtcctcc

SEQ ID NO: 57 - protein, 120 aa, Artificial Sequence
  Synthetic
QAQLVQSGAEVKKPGASVKVSCKASGYSFTDYYMNWLRQAPGQGLEWIGDINPKDGDSGYSHKFKGRATLTVDKSTSTVYMELRSLRSEDTAVYYCASGFTTVVARGDYWGQGTTVTVSS

SEQ ID NO: 58 - DNA, 360 bp, Artificial Sequence
  Synthetic
caagctcaactggtgcagtccggcgccgaggtgaaaaagcccggtgcctccgtgaaggtgagctgcaaggcctccggctactcctttaccgactactacatgaactggctgaggcaagctcccggtcaaggtctggagtggatcggcgatatcaaccccaaggacggcgactccggctacagccataagttcaagggtcgtgccactttaaccgtggacaagtccaccagcaccgtgtacatggagctgaggtctttaaggtccgaggacaccgccgtgtactactgcgcctccggcttcaccacagtggtggctcgtggcgactattggggccaaggtaccaccgtgaccgtgagctcc

SEQ ID NO: 59 - protein, 112 aa, Artificial Sequence
  Synthetic
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPYTFGGGTKVEIK

SEQ ID NO: 60 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctacctgaattggctgcagcagagaccaggacagtctcctagaagactgatctacctggtgtccaagctggattctggagtgccagatagattttccggctccggctctggcacagatttcaccctgaagatctctagagtggaggcagaagacgtgggagtgtactattgttggcagggaacccacttcccttacacatttggaggaggcacaaaggtggagatcaag

SEQ ID NO: 61 - protein, 112 aa, Artificial Sequence
  Synthetic
DVVMTQSPLSLPVTLGQPASISCKSSQSLLDSDGKTYLNWLQQRPGQSPRRLIYLVSKLDSGFPDRFSGSGSGTDFTLKISRVEAEDVGVYYCWQGTHFPYTFGGGTKVEIK

SEQ ID NO: 62 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctacctgaattggctgcagcagagaccaggacagtctcctagaagactgatctacctggtgtccaagctggattctggattcccagatagattttccggctccggctctggcacagatttcaccctgaagatctctagagtggaggcagaagacgtgggagtgtactattgttggcagggaacccacttcccttacacatttggaggaggcacaaaggtggagatcaag

SEQ ID NO: 63 - protein, 21 aa, Artificial Sequence
  Synthetic
NSFYIPRHIRKEEGSFQSCSF

SEQ ID NO: 64 - protein, 21 aa, Artificial Sequence
  Synthetic
FSYSVPNTFPQSTESLVHCDS

SEQ ID NO: 65 - protein, 17 aa, Artificial Sequence
  Synthetic
MGWSCIILFLVATGVHS

SEQ ID NO: 66 - protein, 184 aa, Artificial Sequence
  Synthetic
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 67 - protein, 184 aa, Artificial Sequence
  Synhtetic
MNRTAYTVGALLLLLGTLLPTAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 68 - protein, 184 aa, Artificial Sequence
  Synthetic
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCFSYSVPNTFPQSTESLVHCDSCKPKKFTTMMVTLNCPELQPPTKKKRVTRVKQCRCISIDLD

SEQ ID NO: 69 - protein, 160 aa, Artificial Sequence
  Synthetic
KKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 70 - protein, 160 aa, Artificial Sequence
  Synthetic
KKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 71 - protein, 24 aa, Artificial Sequence
  Synthetic
MSRTAYTVGALLLLLGTLLPAAEG

SEQ ID NO: 72 - protein, 24 aa, Artificial Sequence
  Synthetic
MNRTAYTVGALLLLLGTLLPTAEG

SEQ ID NO: 73 - protein, 393 aa, Artificial Sequence [sequence omitted]

SEQ ID NO: 74 - protein, 399 aa, Artificial Sequence
  Synthetic
MTAIIKEIVSRNKRRYQEDGFDLDLTYIYPNIIAMGFPAERLEGVYRNNIDDVVRFLDSKHKNHYKIYNLCAERHYDTAKNCRVAQYPFEDHNPPQLELIKPFCEDLDQWLSEDDNHVAAIHCKAGKGRTGVMICAYLLRGKFLKAQEALDFYGEVRTRDKKGVTIPSRRYVYYYSYLLKNHLDYRPVALLFHKMMFETIPMFSGGTCNPQFVVCQLKVKIYSSNSGPTRREDKFMYFEFPQPLPVCGDIKVEFFHKNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKV

SEQ ID NO: 75 - protein, 820 aa, Homo sapiens
SWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR